SEQUENCE LISTING (191 sequences)

SEQ ID NO: 1 - protein, 517 aa, Influenza virus
MKAKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKID

SEQ ID NO: 2 - protein, 518 aa, Influenza virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEID

SEQ ID NO: 3 - protein, 514 aa, Influenza virus
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPDIATRPKVNGQGGRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIK

SEQ ID NO: 4 - protein, 519 aa, Influenza virus
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIESRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIK

SEQ ID NO: 5 - protein, 519 aa, Influenza virus
MKTIIALSYILCLVFTQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIK

SEQ ID NO: 6 - protein, 520 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEIS

SEQ ID NO: 7 - protein, 534 aa, Influenza virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNIT

SEQ ID NO: 8 - protein, 519 aa, Influenza virus
MKTIIALSYILCLVFAQKLPGNDNSTATLCLGHHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIK

SEQ ID NO: 9 - protein, 517 aa, Influenza virus
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGIQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKID

SEQ ID NO: 10 - protein, 535 aa, Influenza virus
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNIT

SEQ ID NO: 11 - protein, 501 aa, Influenza virus
DTICIGYHANNSTDTVDTLLEKNVTVTHSVNLLEDSHNGKLCKLKGIAPLQLGKCNIAGWLLGNPECDSLLPAKSWSYIVETPNSENGACYPGDFIDYEELKEQLSSVSSLERFEIFPKESSWPNHNTLKGVTAACSHRGKSSFYRNLLWLTKTGDSYPKLNNSYVNNKGKEVLVLWGVHHPSSSNEQQSLYHNVNAYVSVVSSNYNRRFTPEIAARPKVRDQPGRMNYYWTLLEPGDTIIFEATGNLIAPWYAFALSRGFGSGIITSNASMHECNTKSQTPQGAINSSLPFQNIHPVPIGECPKYVRSTKLRMVTGLRNIPSIQSRGLFGAIAGFIEGGWTGMIDGWYGYHHQNEQGSGYAADQKSTQNAINGITNKVNSIIEKMNTQFTAVSKEFNNLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKID

SEQ ID NO: 12 - protein, 501 aa, Influenza virus
DTICIGYHANNSTDTVDTVLEKNVTVTHSVNLLEDSHNGKLCRLKGIAPLQLGKCSIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALSRGFGSGIITSNASMDECDTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKID

SEQ ID NO: 13 - protein, 501 aa, Influenza virus
DTICIGYHANNSTDTVDTVLEKNVTVTHSVNLLEDSHNGKLCRLKGIAPLQLGNCSVAGWILGNPKCESLFSKESWSYIAETPNPENGTCYPGYFADYEELREQLSSVSSFERFEIFPKESSWPNHTVTKGVTTSCSHNGKSSFYRNLLWLTEKNGLYPNLSKSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLERRMENLNKKVDDGFLDIWTYNAELLVLLENGRTLDFHDSNVKNLYEKVKSQLKNNAKEIGNGCFEFYHKCNNECMESVKNGTYDYPKYSEESKLNRGKID

SEQ ID NO: 14 - protein, 501 aa, Influenza virus
DTICIGYHANNSTDTVDTVLEKNVTVTHSVNLLEDSHNGKLCRLKGIAPLQLGNCSIAGWILGNPECESLFSKKSWSYIAETPNSENGTCYPGYFADYEELREQLSSVSSFERFEIFPKESSWPNHTVTKGVTASCSHKGRSSFYRNLLWLTKKNGSYPNLSKSYVNNKEKEVLVLWGVHHPSNIGDQRAIYHTENAYVSVVSSHYNRRFTPEIAKRPKVRDQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKID

SEQ ID NO: 15 - protein, 501 aa, Influenza virus
DTICIGYHANNSTDTVDTVLEKNVTVTHSVNLLEDNHNGKLCKLKGIAPLQLGKCSIAGWILGNPECESLFSKKSWSYIAETPNSENGTCYPGYFADYEELREQLSSVSSFERFEIFPKESSWPKHNVTKGVTASCSHKGKSSFYRNLLWLTEKNGSYPNLSKSYVNNKEKEVLVLWGVHHPSNIEDQKTIYRKENAYVSVVSSHYNRRFTPEIAKRPKVRNQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSIIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKID

SEQ ID NO: 16 - protein, 501 aa, Influenza virus
DTICIGYHANNSTDTVDTVLEKNVTVTHSVNLLEDSHNGKLCRLKGIAPLQLGKCNIAGWILGNPECESLLSNRSWSYIAETPNSENGICYPGDFADYEELREQLSSVSSFERFEIFPKESSWPKHNITRGVTVACSHAKKSSFYKNLLWLTEANGLYPSLSKSYVNDREKEVLVLWGVHHPSNIEDQRTLYRKENAYVSVVSSNYNRRFTPEIAERPKVRGQPGRMNYYWTLLEPGDKIIFEANGNLIAPWYAFALSRGPGSGIITSNASMDECDTKCQTPQGAINSSLPFQNIHPVTIGECPKYVRSTKLRMVTGLRNIPSIQSRGLFGAIAGFIEGGWTGMVDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKNQLRNNAKEIGNGCFEFYHKCDNECMESVKNGTYDYPKYSEESKLNRAKID

SEQ ID NO: 17 - protein, 500 aa, Influenza virus
DTICIGYHANNSTDTVDTVLEKNVTVTHSVNLLEDSHNGKLCRLKGIAPLQLGKCNIAGWILGNPECESLLSERSWSYIVETPNSENGTCYPGDFIDYEELREQLSSVSSFERFEIFSKESSWPKHTTGGVTAACSHAGKSSFYRNLLWLTEKDGSYPNLNNSYVNKKGKEVLVLWGVHHPSNIKDQQTLYQKENAYVSVVSSNYNRRFTPEIAERPKVRGQAGRINYYWTLLKPGDTIMFEANGNLIAPWYAFALSRGFGSGIITSNASMHECDTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKNQLRNNAKEIGNGCFEFYHKCNNECMESVKNGTYDYPKYSEESKLNREKID

SEQ ID NO: 18 - protein, 501 aa, Influenza virus
DTICIGYHANNSTDTVDTVLEKNVTVTHSVNLLEDSHNGKLCRLKGIAPLQLGKCSIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWYAFALSRGFGSGIITSNASMDECDTKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKID

SEQ ID NO: 19 - protein, 501 aa, Influenza virus
DTICIGYHANNSTDTVDTVLEKNVTVTHSVNLLEDSHNGKLCRLKGIAPLQLGKCNIAGWILGNPECESLLSKRSWSYIAETPNSENGACYPGDFADYEELREQLSSVSSFERFEIFPKERSWPKHNITRGVTAACSHAGKSSFYKNLLWLTETDGSYPKLSKSYVNNKEKEVLVLWGVHHPSNIEDQKTLYRKENAYVSVVSSNYNRRFTPEIAERPKVRGQAGRINYYWTLLEPGDTIIFEANGNLIAPWYAFALSRDFGSGIITSNASMDECDTKCQTPQGAINSSLPFQNIHPVTIGECPKYVKSTKLRMVTGLRNIPSIQSRGLFGAIAGFIEGGWTGMIDGWYGYHHQNEQGSGYAADQKSTQNAINWITNKVNSVIEKMNTQFTAVGKEFNKLEKRMENLNKKVDDGFLDIWTYNAELLVLLENERTLDFHDSNVKNLYEKVKNQLRNNAKEIGNGCFEFYHKCNNECMESVKNGTYDYPKYSEESKLNREKID

SEQ ID NO: 20 - protein, 500 aa, Influenza virus
DTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKID

SEQ ID NO: 21 - protein, 501 aa, Influenza virus
DTICIGYHANNSTDTVDTVLEKNVTVTHSVNLLEDSHNGKLCRLKGIAPLQLGKCSIAGWILGNPECESLFSKKSWSYIAETPNSENGTCYPGYFADYEELREQLSSVSSFERFEIFPKESSWPKHNVTRGVTASCSHKGKCSFYRNLLWLTEKNGSYPNLSKSYVNNKEKEVLVLWGVHHPSNIEDQKTIYRKENAYVSVVSSHYNRRFTPEIAKRPKVRDQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKID

SEQ ID NO: 22 - protein, 501 aa, Influenza virus
DTICIGYHANNSTDTVDTVLEKNVTVTHSVNLLEDSHNGKLCRLKGIAPLQLGKCNIAGWILGNPECESLFSKKSWSYIAETPNSENGTCYPGYFADYEELREQLSSVSSFERFEIFPKERSWPKHNITRGVTAACSHKGKSSFYRNLLWLTEKNGSYPNLNKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRGFGSGIITSNASMDECDTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKID

SEQ ID NO: 23 - protein, 504 aa, Influenza virus
DQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGVHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSCYSADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEIS

SEQ ID NO: 24 - protein, 503 aa, Influenza virus
DHICIGYHANNSTEQVDTIMEKNVTVTHAQDILEKTHNGKLCDLNGVKPLILKDCSVAGWLLGNPMCDEFINVPEWSYIVEKANPANDLCYPGNFNDYEELKHLLSRINHFEKIQIIPKDSWSDHEASLGVSSACPYQGNSSFFRNVVWLIKKGNAYPTIKKSYNNTNQEDLLVLWGIHHPNDEAEQTRLYQNPTTYISIGTSTLNQRLVPKIATRSKVNGQSGRIDFFWTILKPNDAINFESNGNFIAPEYAYKIVKKGDSTIMKSEVEYGNCNTRCQTPMGAINSSMPFHNIHPLTIGECPKYVKSNKLVLATGLRNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEIS

SEQ ID NO: 25 - protein, 503 aa, Influenza virus
DQICVGYHANNSTEQVDTIMEKNVTVTHAQDILEKTHNGKLCNLDGVKPLILKDCSVAGWLLGNPMCDEFLNVSEWSYIVEKASPANGLCYPGDFNDYEELKHLLSRINHFEKIKIIPKSSWSNHEASGVSSACSYLGKPSFFRNLVWLIKKNNTYPPIKVNYTNTNQEDLLVLWGIHHPNDETEQVKIYQNPTTYISVGTSTLNQRLVPKIATRSKVNGQSGRMEFFWTILKPNDAINFDSNGNFIAPEYAYKIVKKGDSAIMKSELEYGNCNTKCQTPMGAINSSMPFHNIHPLTIGECPKYVKSNRLVLATGLRNAPQREGGRRKRGLFGAIAGFIEGGWQGMVDGWYGYHHSNEQGSGYAADKESTQKAIDGITNKVNSIIDKMNTQFEAVGREFNNLERRIENLNKKMEDGFLDVWTYNAELLVLMENERTLDFHDSNVKNLYEKVRLQLRDNAKELGNGCFEFYHKCDNECMESVKNGTYDYPQYSEEARLNREEIS

SEQ ID NO: 26 - protein, 504 aa, Influenza virus
DQICIGYHANNSTEQVDTIMEKNVTVTHAQDILEKTHNGKLCDLDGVKPLILRDCSVAGWLLGNPMCDEFINVPEWSYIVEKASPANDLCYPGDFNDYEELKHLLSRINHFEKIQIIPKSSWSNHEASSGVSSACPYLGKSSFFRNVVWLIKKNSTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEIS

SEQ ID NO: 27 - protein, 504 aa, Influenza virus
DQICIGYHANNSTEQVDTIMEKNVAVTHAQDILEKTHNGKLCDLDGVKPLILRDCSVAGWLLGNPMCDEFINVPEWSYIVEKASPANGLCYPGDFNDYEELKHLLSRINHFEKIQIIPKSSWSNHEASSGVSSACPYQGKSSFFRNVVWLIKKNSTYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEIS

SEQ ID NO: 28 - protein, 503 aa, Influenza virus
QKLPGNDNSTATLCLGHHAVPNGTIVKTITNDQIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTQLNFKYPALNVTMPNNEQFDKLYIWGVHHPVTDKDQIFLYAQSSGRITVSTKRSQQAVIPNIGYRPRIRNIPSRISIYWTIVKPGDILLINSTGNLIAPRGYFKIRSGKSSIMRSDAPIGKCNSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIK

SEQ ID NO: 29 - protein, 532 aa, Influenza virus
DRICTGITSSNSPHVVKTATQGEVNVTGVIPLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHT

SEQ ID NO: 30 - protein, 533 aa, Influenza virus
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHT

SEQ ID NO: 31 - protein, 503 aa, Influenza virus
QKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWNGVTQNGTSSACIRRSNNSFFSRLNWLTHLNFKYPALNVTMPNNEQFDKLYIWGVHHPVTDKDQIFLYAQPSGRITVSTKRSQQAVIPNIGFRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIK

SEQ ID NO: 32 - protein, 213 aa, Influenza virus
IAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRG

SEQ ID NO: 33 - protein, 213 aa, Influenza virus
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRG

SEQ ID NO: 34 - protein, 214 aa, Influenza virus
VAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERN

SEQ ID NO: 35 - protein, 214 aa, Influenza virus
VAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERN

SEQ ID NO: 36 - protein, 213 aa, Influenza virus
IPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPDIATRPKVNGQGGRMEFSWTLLDMWDTINFESTGNLIAPEYGFKISKRG

SEQ ID NO: 37 - protein, 209 aa, Influenza virus
HRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIESRPWVRGLSSRISIYWTIVKPGDVLVINSNGNLIAPRGYFKMRTGK

SEQ ID NO: 38 - protein, 209 aa, Influenza virus
HQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGK

SEQ ID NO: 39 - protein, 232 aa, Influenza virus
RTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRS

SEQ ID NO: 40 - protein, 209 aa, Influenza virus
HQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGK

SEQ ID NO: 41 - protein, 214 aa, Influenza virus
IAPLQLGKCNIAGWLLGNPECDSLLPAKSWSYIVETPNSENGACFPGDFIDYEELKEQLSSVSSLERFEIFPKESSWPNHNTLKGVTAACSHRGKSSFYRNLLWLTKTGDSYPKLNNSYVNNKGKEVLVLWGVHHPSSSNEQQSLYHNVNAYVSVVSSNYNRRFTPEIAARPKVRDQPGRMNYYWTLLEPGDTIIFEATGNLIAPWYAFALSRG

SEQ ID NO: 42 - protein, 214 aa, Influenza virus
IAPLQLGKCSIAGWILGNPECESLFSKKSWSYIAETPNSENGTCFPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALSRG

SEQ ID NO: 43 - protein, 214 aa, Influenza virus
IAPLQLGNCSVAGWILGNPKCESLFSKESWSYIAETPNPENGTCFPGYFADYEELREQLSSVSSFERFEIFPKESSWPNHTVTKGVTTSCSHNGKSSFYRNLLWLTEKNGLYPNLSKSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRG

SEQ ID NO: 44 - protein, 214 aa, Influenza virus
IAPLQLGNCSIAGWILGNPECESLFSKKSWSYIAETPNSENGTCFPGYFADYEELREQLSSVSSFERFEIFPKESSWPNHTVTKGVTASCSHKGRSSFYRNLLWLTKKNGSYPNLSKSYVNNKEKEVLVLWGVHHPSNIGDQRAIYHTENAYVSVVSSHYNRRFTPEIAKRPKVRDQEGRINYYWTLLEPGDTIIFEANGNLIAPWYAFALSRG

SEQ ID NO: 45 - protein, 214 aa, Influenza virus
IAPLQLGKCSIAGWILGNPECESLFSKKSWSYIAETPNSENGTCFPGYFADYEELREQLSSVSSFERFEIFPKESSWPKHNVTKGVTASCSHKGKSSFYRNLLWLTEKNGSYPNLSKSYVNNKEKEVLVLWGVHHPSNIEDQKTIYRKENAYVSVVSSHYNRRFTPEIAKRPKVRNQEGRINYYWTLLEPGDTIIFEANGNLIAPWYAFALSRG

SEQ ID NO: 46 - protein, 214 aa, Influenza virus
IAPLQLGKCNIAGWILGNPECESLLSNRSWSYIAETPNSENGICFPGDFADYEELREQLSSVSSFERFEIFPKESSWPKHNITRGVTVACSHAKKSSFYKNLLWLTEANGLYPSLSKSYVNDREKEVLVLWGVHHPSNIEDQRTLYRKENAYVSVVSSNYNRRFTPEIAERPKVRGQPGRMNYYWTLLEPGDKIIFEANGNLIAPWYAFALSRG

SEQ ID NO: 47 - protein, 213 aa, Influenza virus
IAPLQLGKCNIAGWILGNPECESLLSERSWSYIVETPNSENGTCFPGDFIDYEELREQLSSVSSFERFEIFSKESSWPKHTTGGVTAACSHAGKSSFYRNLLWLTEKDGSYPNLNNSYVNKKGKEVLVLWGVHHPSNIKDQQTLYQKENAYVSVVSSNYNRRFTPEIAERPKVRGQAGRINYYWTLLKPGDTIMFEANGNLIAPWYAFALSRG

SEQ ID NO: 48 - protein, 214 aa, Influenza virus
IAPLQLGKCSIAGWILGNPECESLFSKKSWSYIAETPNSENGTCFPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWYAFALSRG

SEQ ID NO: 49 - protein, 214 aa, Influenza virus
IAPLQLGKCNIAGWILGNPECESLLSKRSWSYIAETPNSENGACFPGDFADYEELREQLSSVSSFERFEIFPKERSWPKHNITRGVTAACSHAGKSSFYKNLLWLTETDGSYPKLSKSYVNNKEKEVLVLWGVHHPSNIEDQKTLYRKENAYVSVVSSNYNRRFTPEIAERPKVRGQAGRINYYWTLLEPGDTIIFEANGNLIAPWYAFALSRD

SEQ ID NO: 50 - protein, 213 aa, Influenza virus
IAPLQLGNCSVAGWILGNPECELLISKESWSYIVEKPNPENGTCFPGHFADYEELREQLSSVSSFERFEIFPKESSWPNHTVTGVSASCSHNGESSFYRNLLWLTGKNGLYPNLSKSYANNKEKEVLVLWGVHHPPNIGIQKALYHTENAYVSVVSSHYSRKFTPEIAKRPKVRDQEGRINYYWTLLEPGDTIIFEANGNLIAPRYAFALSRG

SEQ ID NO: 51 - protein, 214 aa, Influenza virus
IAPLQLGKCSIAGWILGNPECESLFSKKSWSYIAETPNSENGTCFPGYFADYEELREQLSSVSSFERFEIFPKESSWPKHNVTRGVTASCSHKGKCSFYRNLLWLTEKNGSYPNLSKSYVNNKEKEVLVLWGVHHPSNIEDQKTIYRKENAYVSVVSSHYNRRFTPEIAKRPKVRDQEGRINYYWTLLEPGDTIIFEANGNLIAPWYAFALSRG

SEQ ID NO: 52 - protein, 214 aa, Influenza virus
IAPLQLGKCNIAGWILGNPECESLFSKKSWSYIAETPNSENGTCFPGYFADYEELREQLSSVSSFERFEIFPKERSWPKHNITRGVTAACSHKGKSSFYRNLLWLTEKNGSYPNLNKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRG

SEQ ID NO: 53 - protein, 213 aa, Influenza virus
VKPLILRDCSVAGWLLGNPMCDEFINVPEWSYIVEKANPTNDLCFPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKK

SEQ ID NO: 54 - protein, 213 aa, Influenza virus
VKPLILRDCSVAGWLLGNPMCDEFINVPEWSYIVEKASPANDLCFPGNFNDYEELKHLLSRINHFEKIQIIPKSSWSNHDASSGVSSACPYLGRSSFFRNVVWLIKKNSAYPTIKRSYNNTNQEDLLVLWGVHHPNDAAEQTKLYQNPTTYISVGTSTLNQRLVPEIATRPKVNGQSGRMEFFWTILKPNDAINFESNGNFIAPEYAYKIVKK

SEQ ID NO: 55 - protein, 213 aa, Influenza virus
VKPLILKDCSVAGWLLGNPMCDEFINVPEWSYIVEKANPANDLCFPGNFNDYEELKHLLSRINHFEKIQIIPKDSWSDHEASLGVSSACPYQGNSSFFRNVVWLIKKGNAYPTIKKSYNNTNQEDLLVLWGIHHPNDEAEQTRLYQNPTTYISIGTSTLNQRLVPKIATRSKVNGQSGRIDFFWTILKPNDAINFESNGNFIAPEYAYKIVKK

SEQ ID NO: 56 - protein, 212 aa, Influenza virus
VKPLILKDCSVAGWLLGNPMCDEFLNVSEWSYIVEKASPANGLCFPGDFNDYEELKHLLSRINHFEKIKIIPKSSWSNHEASGVSSACSYLGKPSFFRNLVWLIKKNNTYPPIKVNYTNTNQEDLLVLWGIHHPNDETEQVKIYQNPTTYISVGTSTLNQRLVPKIATRSKVNGQSGRMEFFWTILKPNDAINFDSNGNFIAPEYAYKIVKK

SEQ ID NO: 57 - protein, 213 aa, Influenza virus
VKPLILRDCSVAGWLLGNPMCDEFINVPEWSYIVEKASPANDLCFPGDFNDYEELKHLLSRINHFEKIQIIPKSSWSNHEASSGVSSACPYLGKSSFFRNVVWLIKKNSTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKK

SEQ ID NO: 58 - protein, 213 aa, Influenza virus
VKPLILRDCSVAGWLLGNPMCDEFINVPEWSYIVEKASPANGLCFPGDFNDYEELKHLLSRINHFEKIQIIPKSSWSNHEASSGVSSACPYQGKSSFFRNVVWLIKKNSTYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKK

SEQ ID NO: 59 - protein, 208 aa, Influenza virus
HQILDGENCTLIDALLGDPQCDGFQNKKWDLFVERSKAYSNCFPYDVPDYASLRSLVASSGTLEFNNESFNWTGVTQNGTSSACIRRSNNSFFSRLNWLTQLNFKYPALNVTMPNNEQFDKLYIWGVHHPVTDKDQIFLYAQSSGRITVSTKRSQQAVIPNIGYRPRIRNIPSRISIYWTIVKPGDILLINSTGNLIAPRGYFKIRSG

SEQ ID NO: 60 - protein, 231 aa, Influenza virus
KTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGR

SEQ ID NO: 61 - protein, 232 aa, Influenza virus
ETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGR

SEQ ID NO: 62 - protein, 208 aa, Influenza virus
QILDGENCTLIDALLGDPQCDGFQNKKWDLFVERSKAYSNCYPYDVPDYASLRSLVASSGTLEFNNESFNWNGVTQNGTSSACIRRSNNSFFSRLNWLTHLNFKYPALNVTMPNNEQFDKLYIWGVHHPVTDKDQIFLYAQPSGRITVSTKRSQQAVIPNIGFRPRIRNIPSRISIYWTIVKPGDILLINSTGNLIAPRGYFKIRSGK

SEQ ID NO: 63 - DNA, 504 bp, Helicobacter pylori
  source 1..504: mol_type = genomic DNA
atgttatcaaaagacatcattaagttgctaaacgaacaagtgaataaggaaatgaactcttccaacttgtatatgagcatgagttcatggtgctatacccatagcttagatggcgcggggcttttcttgtttgaccatgcggctgaagaatacgagcatgctaaaaagcttattatcttcttgaatgaaaacaatgtgcctgtgcaattgaccagcatcagcgcgcctgagcataagtttgaaggtttgactcaaattttccaaaaagcctatgaacatgagcaacacatcagcgagtctattaacaatatcgtagatcacgccataaaaagcaaagatcatgcgactttcaatttcttgcaatggtatgtggctgaacagcatgaagaagaagtgcttttcaaggatattttggataaaattgagttgattggtaatgaaaaccatggcttgtatttagccgatcagtatgtcaaagggatcgctaaaagcaggaaatcttaa

SEQ ID NO: 64 - protein, 167 aa, Helicobacter pylori
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 65 - DNA, 504 bp, Helicobacter pylori
  source 1..504: mol_type = genomic DNA
ttaagatttcctgcttttagcgatccctttgacatactgatcggctaaatacaagccatggttttcattaccaatcaactcaattttatccaaaatatccttgaaaagcacttcttcttcatgctgttcagccacataccattgcaagaaattgaaagtcgcatgatctttgctttttatggcgtgatctacgatattgttaatagactcgctgatgtgttgctcatgttcataggctttttggaaaatttgagtcaaaccttcaaacttatgctcaggcgcgctgatgctggtcaattgcacaggcacattgttttcattcaagaagataataagctttttagcatgctcgtattcttcagccgcatggtcaaacaagaaaagccccgcgccatctaagctatgggtatagcaccatgaactcatgctcatatacaagttggaagagttcatttccttattcacttgttcgtttagcaacttaatgatgtcttttgataacat

SEQ ID NO: 66 - skipped

SEQ ID NO: 67 - protein, 165 aa, Escherichia coli
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 68-69 - skipped (2 entries)

SEQ ID NO: 70 - protein, 173 aa, Rana catesbeiana
MESQVRQNFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 71 - skipped

SEQ ID NO: 72 - DNA, 531 bp, synthetic construct
  misc_feature 1..531: note = Synthetic
  source 1..531: mol_type = other DNA
tccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcc

SEQ ID NO: 73 - protein, 177 aa, synthetic construct
  REGION 1..177: note = Synthetic
SGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 74 - DNA, 531 bp, synthetic construct
  misc_feature 1..531: note = Synthetic
  source 1..531: mol_type = other DNA
ggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccgga

SEQ ID NO: 75 - DNA, 516 bp, synthetic construct
  misc_feature 1..516: note = Synthetic
  source 1..516: mol_type = other DNA
tccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcc

SEQ ID NO: 76 - protein, 172 aa, synthetic construct
  REGION 1..172: note = Synthetic
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 77 - DNA, 516 bp, synthetic construct
  misc_feature 1..516: note = Synthetic
  source 1..516: mol_type = other DNA
ggatccgtccagggtgctcagctccttgtcgatgaagtacaggccctcgccgctcttgccggccaggctcagcttgtcgatgatgctcttgaacagcttctcctcctcgtgctgctcgctcacgtaccactgcaggaagttgaaggtggggtagtcctggttggtcatggcggcgtgggccagctcgttgatcttctgggtgatcagctgctcgtgcttgtaggtctcctggaacagctcgtccaggctgctgtactcggcgaaggggctctccacggtgttgatcctgggcaggttgccggtgtcggtcaggtagtcgaacagcctctgcatgtgggtcatctcctcctgggcgtgcctcctcaggaaggcggcggcgccctcgaaggtgtggtagctgcaccaggcgctcatctgctggtacagcaggctgctgtacagctccaggttcatctgctcgttcagcttctcctccatctcgggcttgaagttctgcctcacctggctctctccgga

SEQ ID NO: 78 - DNA, 792 bp, Thermotoga maritima
  source 1..792: mol_type = genomic DNA
atggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaag

SEQ ID NO: 79 - protein, 264 aa, Thermotoga maritima
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLK

SEQ ID NO: 80 - DNA, 792 bp, Thermotoga maritima
  source 1..792: mol_type = genomic DNA
cttcagcaggatcagggcctcggggttcaccacctggaaggtgaaggtctcggtgatgaacagcctcacggcgtccttctccctgtcctcgtagccgatgctcaggtcctggcccaggatcagcttgaagtcgccgcccctctcgctcaccaccagggcgtcctcgatcctgggggtggtgatgatcttgccgcccctcaggcactcctccaccctcttctccagggggtagtggccggcctcctccttcaggaagttgatccacctgtcggtgttgatcaccagggtgtaggggccctcgatgccgtccttgctgaagatgctcagggccctcacgatggcctccagcaggtccttgggggtgctgccgcactcgatcttcctctcctcgaagctcagcaggcccttcacgccgctcttctcgcagcccctgaagatcacctcgtcctcgaactcggccaccttcctcacggtctcctccaggctgctcaggtccacgttgggcttgcccctctccaggttgtccagctcccacaggtccagggtgaaggtggccctcagctcgatcaggggcaggctcttcctcaggccccacttcaccacctcgttctcgtcgctcagcacctccacctcgcccagggggtgggcggcgtactcccagccgtaggggccctccacgtccacgaacttcctgccgtacagctgggtcttgaagatctccctggccctgttgtcgatctcctgccactgcttctcggtcagaggggcgaagctcctcttcaggaactccat

SEQ ID NO: 81 - skipped

SEQ ID NO: 82 - protein, 192 aa, synthetic construct
  REGION 1..192: note = Synthetic
MSQAIGILELTSIAAGMELGDAMLKSANVDLLVSKTISPGKFLLMLGGDIGAIQQAIETGTSQAGELLVDSLVLANIHPSVLPAISGLNSVDKRQAVGIVETWSVAACISAADRAVKGSNVTLVRVHMAFGIGGKCYMVVAGDVSDVALAVTVASSSAGAYGLLVYASLIPRPHEAMWRQMVEGLEHHHHHH

SEQ ID NO: 83-84 - skipped (2 entries)

SEQ ID NO: 85 - protein, 318 aa, Acidianus ambivalens
  SITE 31: note = misc_feature - X can be any naturally occurring amino acid
MPKPYVAINMAELKNEPKTFEMFASVGPKVXMVTARHPGFVGFQNHIQIGILPFGNRYGGAKMDMTKESSTVRVLQYTFWKDWKDHEEMHRQNWSYLFRLCYSCASQMIWGPWEPIYEIIYANMPINTEMTDFTAVVGKKFAEGKPLDIPVISQPYGKRVVAFAEHSVIPGKEKQFEDAIVRTLEMLKKAPGFLGAMVLKEIGVSGIGSMQFGAKGFHQVLENPGSLEPDPNNVMYSVPEAKNTPQQYIVHVEWANTDALMFGMGRVLLYPELRQVHDEVLDTLVYGPYIRILNPMMEGTFWREYLNEQAWRHPQFGG

SEQ ID NO: 86-87 - skipped (2 entries)

SEQ ID NO: 88 - protein, 154 aa, Aquifex aeolicus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 89-90 - skipped (2 entries)

SEQ ID NO: 91 - protein, 242 aa, Bacillus stearothermophilus
AAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLM

SEQ ID NO: 92 - skipped

SEQ ID NO: 93 - DNA, 3747 bp, Chikungunya virus
  source 1..3747: mol_type = genomic DNA
atggagttcatcccgacgcaaactttctataacagaaggtaccaaccccgaccctgggccccacgccctacaattcaagtaattagacctagaccacgtccacagaggcaggctgggcaactcgcccagctgatctccgcagtcaacaaattgaccatgcgcgcggtacctcaacagaagcctcgcagaaatcggaaaaacaagaagcaaaggcagaagaagcaggcgccgcaaaacgacccaaagcaaaagaagcaaccaccacaaaagaagccggctcaaaagaagaagaaaccaggccgtagggagagaatgtgcatgaaaattgaaaatgattgcatcttcgaagtcaagcatgaaggcaaagtgatgggctacgcatgcctggtgggggataaagtaatgaaaccagcacatgtgaagggaactatcgacaatgccgatctggctaaactggcctttaagcggtcgtctaaatacgatcttgaatgtgcacagataccggtgcacatgaagtctgatgcctcgaagtttacccacgagaaacccgaggggtactataactggcatcacggagcagtgcagtattcaggaggccggttcactatcccgacgggtgcaggcaagccgggagacagcggcagaccgatcttcgacaacaaaggacgggtggtggccatcgtcctaggaggggccaacgaaggtgcccgcacggccctctccgtggtgacgtggaacaaagacatcgtcacaaaaattacccctgagggagccgaagagtggagcctcgccctcccggtcttgtgcctgttggcaaacactacattcccctgctctcagccgccttgcacaccctgctgctacgaaaaggaaccggaaagcaccttgcgcatgcttgaggacaacgtgatgagacccggatactaccagctactaaaagcatcgctgacttgctctccccaccgccaaagacgcagtactaaggacaattttaatgtctataaagccacaagaccatatctagctcattgtcctgactgcggagaagggcattcgtgccacagccctatcgcattggagcgcatcagaaatgaagcaacggacggaacgctgaaaatccaggtctctttgcagatcgggataaagacagatgacagccacgattggaccaagctgcgctatatggatagccatacgccagcggacgcggagcgagccggattgcttgtaaggacttcagcaccgtgcacgatcaccgggaccatgggacactttattctcgcccgatgcccgaaaggagagacgctgacagtgggatttacggacagcagaaagatcagccacacatgcacacacccgttccatcatgaaccacctgtgataggtagggagaggttccactctcgaccacaacatggtaaagagttaccttgcagcacgtacgtgcagagcaccgctgccactgctgaggagatagaggtgcatatgcccccagatactcctgaccgcacgctgatgacgcagcagtctggcaacgtgaagatcacagttaatgggcagacggtgcggtacaagtgcaactgcggtggctcaaacgagggactgacaaccacagacaaagtgatcaataactgcaaaattgatcagtgccatgctgcagtcactaatcacaagaattggcaatacaactcccctttagtcccgcgcaacgctgaactcggggaccgtaaaggaaagatccacatcccattcccattggcaaacgtgacttgcagagtgccaaaagcaagaaaccctacagtaacttacggaaaaaaccaagtcaccatgctgctgtatcctgaccatccgacactcttgtcttaccgtaacatgggacaggaaccaaattaccacgaggagtgggtgacacacaagaaggaggttaccttgaccgtgcctactgagggtctggaggtcacttggggcaacaacgaaccatacaagtactggccgcagatgtctacgaacggtactgctcatggtcacccacatgagataatcttgtactattatgagctgtaccccactatgactgtagtcattgtgtcggtggcctcgttcgtgcttctgtcgatggtgggcacagcagtgggaatgtgtgtgtgcgcacggcgcagatgcattacaccatatgaattaacaccaggagccactgttcccttcctgctcagcctgctatgctgcgtcagaacgaccaaggcggccacatattacgaggctgcggcatatctatggaacgaacagcagcccctgttctggttgcaggctcttatcccgctggccgccttgatcgtcctgtgcaactgtctgaaactcttgccatgctgctgtaagaccctggcttttttagccgtaatgagcatcggtgcccacactgtgagcgcgtacgaacacgtaacagtgatcccgaacacggtgggagtaccgtataagactcttgtcaacagaccgggttacagccccatggtgttggagatggagctacaatcagtcaccttggaaccaacactgtcacttgactacatcacgtgcgagtacaaaactgtcatcccctccccgtacgtgaagtgctgtggtacagcagagtgcaaggacaagagcctaccagactacagctgcaaggtctttactggagtctacccatttatgtggggcggcgcctactgcttttgcgacgccgaaaatacgcaattgagcgaggcacatgtagagaaatctgaatcttgcaaaacagagtttgcatcggcctacagagcccacaccgcatcggcgtcggcgaagctccgcgtcctttaccaaggaaacaacattaccgtagctgcctacgctaacggtgaccatgccgtcacagtaaaggacgccaagtttgtcgtgggcccaatgtcctccgcctggacaccttttgacaacaaaatcgtggtgtacaaaggcgacgtctacaacatggactacccaccttttggcgcaggaagaccaggacaatttggtgacattcaaagtcgtacaccggaaagtaaagacgtttatgccaacactcagttggtactacagaggccagcagcaggcacggtacatgtaccatactctcaggcaccatctggcttcaagtattggctgaaggaacgaggagcatcgctacagcacacggcaccgttcggttgccagattgcgacaaacccggtaagagctgtaaattgcgctgtggggaacataccaatttccatcgacataccggatgcggcctttactagggttgtcgatgcaccctctgtaacggacatgtcatgcgaagtaccagcctgcactcactcctccgactttgggggcgtcgccatcatcaaatacacagctagcaagaaaggtaaatgtgcagtacattcgatgaccaacgccgttaccattcgagaagccgacgtagaagtagaggggaactcccagctgcaaatatccttctcaacagccctggcaagcgccgagtttcgcgtgcaagtgtgctccacacaagtacactgcgcagccgcatgccaccctccaaaggaccacatagtcaattacccagcatcacacaccacccttggggtccaggatatatccacaacggcaatgtcttgggtgcagaagattacgggaggagtaggattaattgttgctgttgctgccttaattttaattgtggtgctatgcgtgtcgtttagcaggcactaa

SEQ ID NO: 94 - protein, 1248 aa, Chikungunya virus
MEFIPTQTFYNRRYQPRPWAPRPTIQVIRPRPRPQRQAGQLAQLISAVNKLTMRAVPQQKPRRNRKNKKQRQKKQAPQNDPKQKKQPPQKKPAQKKKKPGRRERMCMKIENDCIFEVKHEGKVMGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLALPVLCLLANTTFPCSQPPCTPCCYEKEPESTLRMLEDNVMRPGYYQLLKASLTCSPHRQRRSTKDNFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKAATYYEAAAYLWNEQQPLFWLQALIPLAALIVLCNCLKLLPCCCKTLAFLAVMSIGAHTVSAYEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 95 - DNA, 3747 bp, Chikungunya virus
  source 1..3747: mol_type = genomic DNA
ttagtgcctgctaaacgacacgcatagcaccacaattaaaattaaggcagcaacagcaacaattaatcctactcctcccgtaatcttctgcacccaagacattgccgttgtggatatatcctggaccccaagggtggtgtgtgatgctgggtaattgactatgtggtcctttggagggtggcatgcggctgcgcagtgtacttgtgtggagcacacttgcacgcgaaactcggcgcttgccagggctgttgagaaggatatttgcagctgggagttcccctctacttctacgtcggcttctcgaatggtaacggcgttggtcatcgaatgtactgcacatttacctttcttgctagctgtgtatttgatgatggcgacgcccccaaagtcggaggagtgagtgcaggctggtacttcgcatgacatgtccgttacagagggtgcatcgacaaccctagtaaaggccgcatccggtatgtcgatggaaattggtatgttccccacagcgcaatttacagctcttaccgggtttgtcgcaatctggcaaccgaacggtgccgtgtgctgtagcgatgctcctcgttccttcagccaatacttgaagccagatggtgcctgagagtatggtacatgtaccgtgcctgctgctggcctctgtagtaccaactgagtgttggcataaacgtctttactttccggtgtacgactttgaatgtcaccaaattgtcctggtcttcctgcgccaaaaggtgggtagtccatgttgtagacgtcgcctttgtacaccacgattttgttgtcaaaaggtgtccaggcggaggacattgggcccacgacaaacttggcgtcctttactgtgacggcatggtcaccgttagcgtaggcagctacggtaatgttgtttccttggtaaaggacgcggagcttcgccgacgccgatgcggtgtgggctctgtaggccgatgcaaactctgttttgcaagattcagatttctctacatgtgcctcgctcaattgcgtattttcggcgtcgcaaaagcagtaggcgccgccccacataaatgggtagactccagtaaagaccttgcagctgtagtctggtaggctcttgtccttgcactctgctgtaccacagcacttcacgtacggggaggggatgacagttttgtactcgcacgtgatgtagtcaagtgacagtgttggttccaaggtgactgattgtagctccatctccaacaccatggggctgtaacccggtctgttgacaagagtcttatacggtactcccaccgtgttcgggatcactgttacgtgttcgtacgcgctcacagtgtgggcaccgatgctcattacggctaaaaaagccagggtcttacagcagcatggcaagagtttcagacagttgcacaggacgatcaaggcggccagcgggataagagcctgcaaccagaacaggggctgctgttcgttccatagatatgccgcagcctcgtaatatgtggccgccttggtcgttctgacgcagcatagcaggctgagcaggaagggaacagtggctcctggtgttaattcatatggtgtaatgcatctgcgccgtgcgcacacacacattcccactgctgtgcccaccatcgacagaagcacgaacgaggccaccgacacaatgactacagtcatagtggggtacagctcataatagtacaagattatctcatgtgggtgaccatgagcagtaccgttcgtagacatctgcggccagtacttgtatggttcgttgttgccccaagtgacctccagaccctcagtaggcacggtcaaggtaacctccttcttgtgtgtcacccactcctcgtggtaatttggttcctgtcccatgttacggtaagacaagagtgtcggatggtcaggatacagcagcatggtgacttggttttttccgtaagttactgtagggtttcttgcttttggcactctgcaagtcacgtttgccaatgggaatgggatgtggatctttcctttacggtccccgagttcagcgttgcgcgggactaaaggggagttgtattgccaattcttgtgattagtgactgcagcatggcactgatcaattttgcagttattgatcactttgtctgtggttgtcagtccctcgtttgagccaccgcagttgcacttgtaccgcaccgtctgcccattaactgtgatcttcacgttgccagactgctgcgtcatcagcgtgcggtcaggagtatctgggggcatatgcacctctatctcctcagcagtggcagcggtgctctgcacgtacgtgctgcaaggtaactctttaccatgttgtggtcgagagtggaacctctccctacctatcacaggtggttcatgatggaacgggtgtgtgcatgtgtggctgatctttctgctgtccgtaaatcccactgtcagcgtctctcctttcgggcatcgggcgagaataaagtgtcccatggtcccggtgatcgtgcacggtgctgaagtccttacaagcaatccggctcgctccgcgtccgctggcgtatggctatccatatagcgcagcttggtccaatcgtggctgtcatctgtctttatcccgatctgcaaagagacctggattttcagcgttccgtccgttgcttcatttctgatgcgctccaatgcgatagggctgtggcacgaatgcccttctccgcagtcaggacaatgagctagatatggtcttgtggctttatagacattaaaattgtccttagtactgcgtctttggcggtggggagagcaagtcagcgatgcttttagtagctggtagtatccgggtctcatcacgttgtcctcaagcatgcgcaaggtgctttccggttccttttcgtagcagcagggtgtgcaaggcggctgagagcaggggaatgtagtgtttgccaacaggcacaagaccgggagggcgaggctccactcttcggctccctcaggggtaatttttgtgacgatgtctttgttccacgtcaccacggagagggccgtgcgggcaccttcgttggcccctcctaggacgatggccaccacccgtcctttgttgtcgaagatcggtctgccgctgtctcccggcttgcctgcacccgtcgggatagtgaaccggcctcctgaatactgcactgctccgtgatgccagttatagtacccctcgggtttctcgtgggtaaacttcgaggcatcagacttcatgtgcaccggtatctgtgcacattcaagatcgtatttagacgaccgcttaaaggccagtttagccagatcggcattgtcgatagttcccttcacatgtgctggtttcattactttatcccccaccaggcatgcgtagcccatcactttgccttcatgcttgacttcgaagatgcaatcattttcaattttcatgcacattctctccctacggcctggtttcttcttcttttgagccggcttcttttgtggtggttgcttcttttgctttgggtcgttttgcggcgcctgcttcttctgcctttgcttcttgtttttccgatttctgcgaggcttctgttgaggtaccgcgcgcatggtcaatttgttgactgcggagatcagctgggcgagttgcccagcctgcctctgtggacgtggtctaggtctaattacttgaattgtagggcgtggggcccagggtcggggttggtaccttctgttatagaaagtttgcgtcgggatgaactccat

SEQ ID NO: 96 - DNA, 1266 bp, synthetic construct
  misc_feature 1..1266: note = Synthetic
  source 1..1266: mol_type = other DNA
atggacagcaagggcagcagccagaagggcagcagactgctgctgctgctggtggtgagcaacctgctgctgcctcagggcgtgctagccattgcccctctgcagctgggcaattgttctgtggccggatggattctgggcaaccccgagtgtgagctgctgatttctaaggagagctggagctacatcgtggagacccccaatcctgagaatggcacctgctaccctggctacttcgccgattacgaggagctgcgcgagcagctgtctagcgtgtccagcttcgagagattcgagatcttccccaaggagtccagctggcctaatcacacagtgacaggcgtgtctgccagctgtagccacaacggcaaaagcagcttctaccggaacctgctgtggctgacaggcaagaatggcctgtaccccaacctgagcaagagctacgtgaacaacaaggaaaaggaagtgctggtgctgtggggagtgcaccaccctcccaacatcggaaatcagcgggccctgtaccacacagagaacgcctatgtgagcgtggtgtccagccactacagcagaagattcacccccgagatcgccaagagacccaaagtgagagaccaggagggccggatcaattactactggaccctgctggagcctggcgataccatcatcttcgaggccaacggcaatctgatcgccccttggtatgcctttgccctgagcagaggcgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 97 - protein, 421 aa, synthetic construct
  REGION 1..421: note = Synthetic
MDSKGSSQKGSRLLLLLVVSNLLLPQGVLAIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 98 - DNA, 1266 bp, synthetic construct
  misc_feature 1..1266: note = Synthetic
  source 1..1266: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgcctctgctcagggcaaaggcataccaaggggcgatcagattgccgttggcctcgaagatgatggtatcgccaggctccagcagggtccagtagtaattgatccggccctcctggtctctcactttgggtctcttggcgatctcgggggtgaatcttctgctgtagtggctggacaccacgctcacataggcgttctctgtgtggtacagggcccgctgatttccgatgttgggagggtggtgcactccccacagcaccagcacttccttttccttgttgttcacgtagctcttgctcaggttggggtacaggccattcttgcctgtcagccacagcaggttccggtagaagctgcttttgccgttgtggctacagctggcagacacgcctgtcactgtgtgattaggccagctggactccttggggaagatctcgaatctctcgaagctggacacgctagacagctgctcgcgcagctcctcgtaatcggcgaagtagccagggtagcaggtgccattctcaggattgggggtctccacgatgtagctccagctctccttagaaatcagcagctcacactcggggttgcccagaatccatccggccacagaacaattgcccagctgcagaggggcaatggctagcacgccctgaggcagcagcaggttgctcaccaccagcagcagcagcagtctgctgcccttctggctgctgcccttgctgtccat

SEQ ID NO: 99 - DNA, 1266 bp, synthetic construct
  misc_feature 1..1266: note = Synthetic
  source 1..1266: mol_type = other DNA
atggacagcaagggcagcagccagaagggcagcagactgctgctgctgctggtggtgagcaacctgctgctgcctcagggcgtgctagccattgcccctctgcagctgggcaattgttctgtggccggatggattctgggcaaccccgagtgtgagctgctgatttctaaggagagctggagctacatcgtggagacccccaatcctgagaatggcacctgcttccctggctacttcgccgattacgaggagctgcgcgagcagctgtctagcgtgtccagcttcgagagattcgagatcttccccaaggagtccagctggcctaatcacacagtgacaggcgtgtctgccagctgtagccacaacggcaaaagcagcttctaccggaacctgctgtggctgacaggcaagaatggcctgtaccccaacctgagcaagagctacgtgaacaacaaggaaaaggaagtgctggtgctgtggggagtgcaccaccctcccaacatcggaaatcagcgggccctgtaccacacagagaacgcctatgtgagcgtggtgtccagccactacagcagaagattcacccccgagatcgccaagagacccaaagtgagagaccaggagggccggatcaattactactggaccctgctggagcctggcgataccatcatcttcgaggccaacggcaatctgatcgccccttggtatgcctttgccctgagcagaggcgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 100 - protein, 421 aa, synthetic construct
  REGION 1..421: note = Synthetic
MDSKGSSQKGSRLLLLLVVSNLLLPQGVLAIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 101 - DNA, 1266 bp, synthetic construct
  misc_feature 1..1266: note = Synthetic
  source 1..1266: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgcctctgctcagggcaaaggcataccaaggggcgatcagattgccgttggcctcgaagatgatggtatcgccaggctccagcagggtccagtagtaattgatccggccctcctggtctctcactttgggtctcttggcgatctcgggggtgaatcttctgctgtagtggctggacaccacgctcacataggcgttctctgtgtggtacagggcccgctgatttccgatgttgggagggtggtgcactccccacagcaccagcacttccttttccttgttgttcacgtagctcttgctcaggttggggtacaggccattcttgcctgtcagccacagcaggttccggtagaagctgcttttgccgttgtggctacagctggcagacacgcctgtcactgtgtgattaggccagctggactccttggggaagatctcgaatctctcgaagctggacacgctagacagctgctcgcgcagctcctcgtaatcggcgaagtagccagggaagcaggtgccattctcaggattgggggtctccacgatgtagctccagctctccttagaaatcagcagctcacactcggggttgcccagaatccatccggccacagaacaattgcccagctgcagaggggcaatggctagcacgccctgaggcagcagcaggttgctcaccaccagcagcagcagcagtctgctgcccttctggctgctgcccttgctgtccat

SEQ ID NO: 102 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccgtggcccccctgcacctgggcaagtgcaacatcgccggctggattctgggcaaccccgagtgcgagagcctgagcaccgccagcagctggagctacatcgtggagacccccagcagcgacaacggcacctgctaccccggcgacttcatcgactacgaggagctgcgggagcagctgagcagcgtgagcagcttcgagcggttcgagatcttccccaagaccagcagctggcccaaccacgacagcaacaagggcgtgaccgccgcctgcccccacgccggcgccaagagcttctacaagaacctgatctggctggtgaagaagggcaacagctaccccaagctgagcaagagctacatcaacgacaagggcaaggaggtgctggtgctgtggggcatccaccaccccagcaccagcgccgaccagcagagcctgtaccagaacgccgacacctacgtgttcgtgggcagcagccggtacagcaagaagttcaagcccgagatcgccatccggcccaaggtgcgggaccaggagggccggatgaactactactggaccctggtggagcccggcgacaagatcaccttcgaggccaccggcaacctggtggtgccccggtacgccttcgccatggagcggaacgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 103 - protein, 422 aa, synthetic construct
  REGION 1..422: note = Synthetic
MDSKGSSQKGSRLLLLLVVSNLLLPQGVLAVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 104 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgttccgctccatggcgaaggcgtaccggggcaccaccaggttgccggtggcctcgaaggtgatcttgtcgccgggctccaccagggtccagtagtagttcatccggccctcctggtcccgcaccttgggccggatggcgatctcgggcttgaacttcttgctgtaccggctgctgcccacgaacacgtaggtgtcggcgttctggtacaggctctgctggtcggcgctggtgctggggtggtggatgccccacagcaccagcacctccttgcccttgtcgttgatgtagctcttgctcagcttggggtagctgttgcccttcttcaccagccagatcaggttcttgtagaagctcttggcgccggcgtgggggcaggcggcggtcacgcccttgttgctgtcgtggttgggccagctgctggtcttggggaagatctcgaaccgctcgaagctgctcacgctgctcagctgctcccgcagctcctcgtagtcgatgaagtcgccggggtagcaggtgccgttgtcgctgctgggggtctccacgatgtagctccagctgctggcggtgctcaggctctcgcactcggggttgcccagaatccagccggcgatgttgcacttgcccaggtgcaggggggccacggctagcacgccctgaggcagcagcaggttgctcaccaccagcagcagcagcagtctgctgcccttctggctgctgcccttgctgtccat

SEQ ID NO: 105 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccgtggcccccctgcacctgggcaagtgcaacatcgccggctggattctgggcaaccccgagtgcgagagcctgagcaccgccagcagctggagctacatcgtggagacccccagcagcgacaacggcacctgcttccccggcgacttcatcgactacgaggagctgcgggagcagctgagcagcgtgagcagcttcgagcggttcgagatcttccccaagaccagcagctggcccaaccacgacagcaacaagggcgtgaccgccgcctgcccccacgccggcgccaagagcttctacaagaacctgatctggctggtgaagaagggcaacagctaccccaagctgagcaagagctacatcaacgacaagggcaaggaggtgctggtgctgtggggcatccaccaccccagcaccagcgccgaccagcagagcctgtaccagaacgccgacacctacgtgttcgtgggcagcagccggtacagcaagaagttcaagcccgagatcgccatccggcccaaggtgcgggaccaggagggccggatgaactactactggaccctggtggagcccggcgacaagatcaccttcgaggccaccggcaacctggtggtgccccggtacgccttcgccatggagcggaacgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 106 - protein, 422 aa, synthetic construct
  REGION 1..422: note = Synthetic
MDSKGSSQKGSRLLLLLVVSNLLLPQGVLAVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 107 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgttccgctccatggcgaaggcgtaccggggcaccaccaggttgccggtggcctcgaaggtgatcttgtcgccgggctccaccagggtccagtagtagttcatccggccctcctggtcccgcaccttgggccggatggcgatctcgggcttgaacttcttgctgtaccggctgctgcccacgaacacgtaggtgtcggcgttctggtacaggctctgctggtcggcgctggtgctggggtggtggatgccccacagcaccagcacctccttgcccttgtcgttgatgtagctcttgctcagcttggggtagctgttgcccttcttcaccagccagatcaggttcttgtagaagctcttggcgccggcgtgggggcaggcggcggtcacgcccttgttgctgtcgtggttgggccagctgctggtcttggggaagatctcgaaccgctcgaagctgctcacgctgctcagctgctcccgcagctcctcgtagtcgatgaagtcgccggggaagcaggtgccgttgtcgctgctgggggtctccacgatgtagctccagctgctggcggtgctcaggctctcgcactcggggttgcccagaatccagccggcgatgttgcacttgcccaggtgcaggggggccacggctagcacgccctgaggcagcagcaggttgctcaccaccagcagcagcagcagtctgctgcccttctggctgctgcccttgctgtccat

SEQ ID NO: 108 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgcccctctgcagctgggcaagtgtaatatcgccggatggctgctgggaaaccccgagtgtgattctctgctgcctgccaagagctggagctacatcgtggagacccccaattctgagaatggcgcctgcttccctggcgacttcatcgattacgaggagctgaaggagcagctgagttctgtctctagcctggagagattcgagatcttccccaaggagagcagctggcccaatcacaataccctgaagggagtgacagccgcctgtagccacagaggcaagagcagcttctaccggaatctgctgtggctgaccaagaccggcgatagctaccccaagctgaacaacagctacgtgaacaacaagggcaaggaagtgctggtgctgtggggagtgcaccaccctagcagcagcaatgagcagcagagcctgtaccacaacgtgaacgcctatgtgagcgtggtgtccagcaactacaaccggagattcacccctgaaatcgccgccagacccaaagtgagagaccagcccggcaggatgaattactactggaccctgctggagcctggcgataccatcatctttgaggccaccggcaatctgattgccccttggtacgcctttgccctgagcagaggcgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 109 - protein, 422 aa, synthetic construct
  REGION 1..422: note = Synthetic
MDSKGSSQKGSRLLLLLVVSNLLLPQGVLAIAPLQLGKCNIAGWLLGNPECDSLLPAKSWSYIVETPNSENGACFPGDFIDYEELKEQLSSVSSLERFEIFPKESSWPNHNTLKGVTAACSHRGKSSFYRNLLWLTKTGDSYPKLNNSYVNNKGKEVLVLWGVHHPSSSNEQQSLYHNVNAYVSVVSSNYNRRFTPEIAARPKVRDQPGRMNYYWTLLEPGDTIIFEATGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 110 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgcctctgctcagggcaaaggcgtaccaaggggcaatcagattgccggtggcctcaaagatgatggtatcgccaggctccagcagggtccagtagtaattcatcctgccgggctggtctctcactttgggtctggcggcgatttcaggggtgaatctccggttgtagttgctggacaccacgctcacataggcgttcacgttgtggtacaggctctgctgctcattgctgctgctagggtggtgcactccccacagcaccagcacttccttgcccttgttgttcacgtagctgttgttcagcttggggtagctatcgccggtcttggtcagccacagcagattccggtagaagctgctcttgcctctgtggctacaggcggctgtcactcccttcagggtattgtgattgggccagctgctctccttggggaagatctcgaatctctccaggctagagacagaactcagctgctccttcagctcctcgtaatcgatgaagtcgccagggaagcaggcgccattctcagaattgggggtctccacgatgtagctccagctcttggcaggcagcagagaatcacactcggggtttcccagcagccatccggcgatattacacttgcccagctgcagaggggcgatggctagcacgccctgaggcagcagcaggttgctcaccaccagcagcagcagcagtctgctgcccttctggctgctgcccttgctgtccat

SEQ ID NO: 111 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccattgcccccctgcagctgggcaagtgcagcattgccggctggattctgggcaaccccgagtgcgagagcctgttcagcaagaagagctggtcctacattgccgagacacccaacagcgagaacggcacctgtttccccggctacttcgccgactacgaggaactgcgggagcagctgagcagcgtgtccagcttcgagcggttcgagatcttccccaaagagcggagctggcccaagcacaacgtgaccagaggcgtgaccgccagctgctctcacaagggcaagagcagcttctaccggaacctgctgtggctgaccgagaagaacggcagctaccccaacctgagcaagagctacgtgaacaacaaagagaaagaggtcctggtcctctggggcgtgcaccaccctagcaacatcgaggaccagaaaaccatctaccggaaagaaaacgcctacgtgtccgtggtgtccagcaactacaaccggcggttcacccccgagatcgccgagaggcctaaagtgcggggccaggccggcagaatcaactactactggaccctgctggaacccggcgacaccatcatcttcgaggccaacggcaacctgatcgccccttggcacgcctttgccctgagcagaggcgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 112 - protein, 422 aa, synthetic construct
  REGION 1..422: note = Synthetic
MDSKGSSQKGSRLLLLLVVSNLLLPQGVLAIAPLQLGKCSIAGWILGNPECESLFSKKSWSYIAETPNSENGTCFPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 113 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgcctctgctcagggcaaaggcgtgccaaggggcgatcaggttgccgttggcctcgaagatgatggtgtcgccgggttccagcagggtccagtagtagttgattctgccggcctggccccgcactttaggcctctcggcgatctcgggggtgaaccgccggttgtagttgctggacaccacggacacgtaggcgttttctttccggtagatggttttctggtcctcgatgttgctagggtggtgcacgccccagaggaccaggacctctttctctttgttgttcacgtagctcttgctcaggttggggtagctgccgttcttctcggtcagccacagcaggttccggtagaagctgctcttgcccttgtgagagcagctggcggtcacgcctctggtcacgttgtgcttgggccagctccgctctttggggaagatctcgaaccgctcgaagctggacacgctgctcagctgctcccgcagttcctcgtagtcggcgaagtagccggggaaacaggtgccgttctcgctgttgggtgtctcggcaatgtaggaccagctcttcttgctgaacaggctctcgcactcggggttgcccagaatccagccggcaatgctgcacttgcccagctgcaggggggcaatggctagcacgccctgaggcagcagcaggttgctcaccaccagcagcagcagcagtctgctgcccttctggctgctgcccttgctgtccat

SEQ ID NO: 114 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgcccctctgcagctgggcaactgcagcgtggctggctggattctggggaatccaaagtgtgagagtctgttttcaaaagaatcttggagttacatcgcagagacccccaaccctgaaaatggaacatgcttccctggctatttcgccgattatgaggaactgagggagcagctgagctccgtgtctagtttcgagcgctttgaaattttcccaaaggaatcaagctggcccaaccacaccgtgacaaaaggcgtcaccacatcatgtagccataacgggaagtcctctttttaccgcaatctgctgtggctgacagagaagaacggcctgtacccaaatctgtccaagtcttacgtgaacaataaggagaaggaagtgctggtcctgtggggcgtccaccatcccagcaacatccgagaccagcgggcaatctaccacacagagaatgcctatgtgagcgtggtcagttcacattacagccggcggttcacccccgagatcgccaagagaccaaaagtgaggggccaggaagggcgaattaactactattggactctgctggagccaggagataccatcattttcgaagccaacggcaatctgatcgctccctggtatgcatttgccctgtcccgcggagcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 115 - protein, 422 aa, synthetic construct
  REGION 1..422: note = Synthetic
MDSKGSSQKGSRLLLLLVVSNLLLPQGVLAIAPLQLGNCSVAGWILGNPKCESLFSKESWSYIAETPNPENGTCFPGYFADYEELREQLSSVSSFERFEIFPKESSWPNHTVTKGVTTSCSHNGKSSFYRNLLWLTEKNGLYPNLSKSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 116 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggctccgcgggacagggcaaatgcataccagggagcgatcagattgccgttggcttcgaaaatgatggtatctcctggctccagcagagtccaatagtagttaattcgcccttcctggcccctcacttttggtctcttggcgatctcgggggtgaaccgccggctgtaatgtgaactgaccacgctcacataggcattctctgtgtggtagattgcccgctggtctcggatgttgctgggatggtggacgccccacaggaccagcacttccttctccttattgttcacgtaagacttggacagatttgggtacaggccgttcttctctgtcagccacagcagattgcggtaaaaagaggacttcccgttatggctacatgatgtggtgacgccttttgtcacggtgtggttgggccagcttgattcctttgggaaaatttcaaagcgctcgaaactagacacggagctcagctgctccctcagttcctcataatcggcgaaatagccagggaagcatgttccattttcagggttgggggtctctgcgatgtaactccaagattcttttgaaaacagactctcacactttggattccccagaatccagccagccacgctgcagttgcccagctgcagaggggcgatggctagcacgccctgaggcagcagcaggttgctcaccaccagcagcagcagcagtctgctgcccttctggctgctgcccttgctgtccat

SEQ ID NO: 117 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccattgcccctctgcagctgggcaattgttctatcgccggctggattctgggaaatcccgagtgcgagagcctgttcagcaagaagtcctggtcctatatcgccgagacacccaacagcgagaatggcacctgtttccctggctacttcgccgattacgaggaactgagagagcagctgtcctctgtctccagcttcgagcggttcgagatcttccccaaagagtccagctggcccaatcacacagtgaccaagggcgtgaccgcctcttgtagccacaagggcagaagcagcttctaccggaacctgctgtggctgaccaagaagaacggcagctaccccaatctgagcaagagctacgtgaacaacaaagaaaaagaggtgctggtcctctggggagtgcaccaccctagcaacatcggagatcagcgggccatctaccacaccgagaacgcctatgtgtccgtggtgtccagccactacaacagaagattcacccccgagatcgccaaaagacccaaagtgcgggaccaggaaggcagaatcaactactactggaccctgctggaacctggcgacaccatcatcttcgaggccaacggcaatctgatcgccccttggtatgcctttgccctgagcagaggcgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 118 - protein, 422 aa, synthetic construct
  REGION 1..422: note = Synthetic
MDSKGSSQKGSRLLLLLVVSNLLLPQGVLAIAPLQLGNCSIAGWILGNPECESLFSKKSWSYIAETPNSENGTCFPGYFADYEELREQLSSVSSFERFEIFPKESSWPNHTVTKGVTASCSHKGRSSFYRNLLWLTKKNGSYPNLSKSYVNNKEKEVLVLWGVHHPSNIGDQRAIYHTENAYVSVVSSHYNRRFTPEIAKRPKVRDQEGRINYYWTLLEPGDTIIFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 119 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgcctctgctcagggcaaaggcataccaaggggcgatcagattgccgttggcctcgaagatgatggtgtcgccaggttccagcagggtccagtagtagttgattctgccttcctggtcccgcactttgggtcttttggcgatctcgggggtgaatcttctgttgtagtggctggacaccacggacacataggcgttctcggtgtggtagatggcccgctgatctccgatgttgctagggtggtgcactccccagaggaccagcacctctttttctttgttgttcacgtagctcttgctcagattggggtagctgccgttcttcttggtcagccacagcaggttccggtagaagctgcttctgcccttgtggctacaagaggcggtcacgcccttggtcactgtgtgattgggccagctggactctttggggaagatctcgaaccgctcgaagctggagacagaggacagctgctctctcagttcctcgtaatcggcgaagtagccagggaaacaggtgccattctcgctgttgggtgtctcggcgatataggaccaggacttcttgctgaacaggctctcgcactcgggatttcccagaatccagccggcgatagaacaattgcccagctgcagaggggcaatggctagcacgccctgaggcagcagcaggttgctcaccaccagcagcagcagcagtctgctgcccttctggctgctgcccttgctgtccat

SEQ ID NO: 120 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgctcctctgcagctggggaaatgcagcatcgcagggtggattctgggaaacccagagtgtgaaagtctgttttcaaagaaatcttggagttacattgccgagacacccaacagcgaaaatggcacttgcttccctgggtatttcgctgattatgaggaactgcgcgagcagctgagctccgtgtctagtttcgagcgatttgaaatcttccccaaggaatcaagctggcctaagcacaacgtgaccaaaggggtcacagcctcatgtagccataagggaaaatcctctttttaccgcaatctgctgtggctgacagagaagaacgggtcctacccaaatctgtccaagtcttacgtgaacaataaggagaaggaagtgctggtcctgtggggcgtccaccatccctctaacatcgaggaccagaagactatctacaggaaagaaaacgcatatgtgagtgtggtcagttcacactacaatcggcggttcacccccgagatcgccaagaggcccaaagtgcgcaaccaggaaggccgcattaattactattggaccctgctggagccaggcgatacaatcattttcgaagccaacgggaatctgatcgctccctggtatgcatttgccctgtcccgaggagcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 121 - protein, 422 aa, synthetic construct
  REGION 1..422: note = Synthetic
MDSKGSSQKGSRLLLLLVVSNLLLPQGVLAIAPLQLGKCSIAGWILGNPECESLFSKKSWSYIAETPNSENGTCFPGYFADYEELREQLSSVSSFERFEIFPKESSWPKHNVTKGVTASCSHKGKSSFYRNLLWLTEKNGSYPNLSKSYVNNKEKEVLVLWGVHHPSNIEDQKTIYRKENAYVSVVSSHYNRRFTPEIAKRPKVRNQEGRINYYWTLLEPGDTIIFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 122 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggctcctcgggacagggcaaatgcataccagggagcgatcagattcccgttggcttcgaaaatgattgtatcgcctggctccagcagggtccaatagtaattaatgcggccttcctggttgcgcactttgggcctcttggcgatctcgggggtgaaccgccgattgtagtgtgaactgaccacactcacatatgcgttttctttcctgtagatagtcttctggtcctcgatgttagagggatggtggacgccccacaggaccagcacttccttctccttattgttcacgtaagacttggacagatttgggtaggacccgttcttctctgtcagccacagcagattgcggtaaaaagaggattttcccttatggctacatgaggctgtgacccctttggtcacgttgtgcttaggccagcttgattccttggggaagatttcaaatcgctcgaaactagacacggagctcagctgctcgcgcagttcctcataatcagcgaaatacccagggaagcaagtgccattttcgctgttgggtgtctcggcaatgtaactccaagatttctttgaaaacagactttcacactctgggtttcccagaatccaccctgcgatgctgcatttccccagctgcagaggagcgatggctagcacgccctgaggcagcagcaggttgctcaccaccagcagcagcagcagtctgctgcccttctggctgctgcccttgctgtccat

SEQ ID NO: 123 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgccccactgcagctgggcaagtgcaacatcgctggctggattctggggaatcccgagtgtgaatcactgctgagcaaccgctcatggagctacatcgctgagacccctaacagcgaaaatggaatttgcttcccaggcgactttgcagattatgaggaactgcgggagcagctgagctccgtgtctagtttcgagagatttgaaatcttccccaaagaatcaagctggcctaagcacaacattaccaggggcgtgacagtcgcctgtagccatgctaagaaatcctctttctacaagaacctgctgtggctgacagaggccaatggcctgtacccctccctgtctaaaagttatgtgaatgaccgcgagaaggaagtgctggtcctgtggggcgtccaccatcctagcaacatcgaggatcagaggacactgtaccgcaaggaaaatgcctatgtgagcgtcgtcagttcaaactacaatcggagatttactccagagattgctgaacgaccaaaagtgcgaggacagcctggacgaatgaactactattggaccctgctggagccaggagataagatcatttttgaagcaaacggcaatctgatcgccccctggtatgcattcgccctgtcaagaggaccttccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 124 - protein, 422 aa, synthetic construct
  REGION 1..422: note = Synthetic
MDSKGSSQKGSRLLLLLVVSNLLLPQGVLAIAPLQLGKCNIAGWILGNPECESLLSNRSWSYIAETPNSENGICFPGDFADYEELREQLSSVSSFERFEIFPKESSWPKHNITRGVTVACSHAKKSSFYKNLLWLTEANGLYPSLSKSYVNDREKEVLVLWGVHHPSNIEDQRTLYRKENAYVSVVSSNYNRRFTPEIAERPKVRGQPGRMNYYWTLLEPGDKIIFEANGNLIAPWYAFALSRGPSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 125 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaaggtcctcttgacagggcgaatgcataccagggggcgatcagattgccgtttgcttcaaaaatgatcttatctcctggctccagcagggtccaatagtagttcattcgtccaggctgtcctcgcacttttggtcgttcagcaatctctggagtaaatctccgattgtagtttgaactgacgacgctcacataggcattttccttgcggtacagtgtcctctgatcctcgatgttgctaggatggtggacgccccacaggaccagcacttccttctcgcggtcattcacataacttttagacagggaggggtacaggccattggcctctgtcagccacagcaggttcttgtagaaagaggatttcttagcatggctacaggcgactgtcacgcccctggtaatgttgtgcttaggccagcttgattctttggggaagatttcaaatctctcgaaactagacacggagctcagctgctcccgcagttcctcataatctgcaaagtcgcctgggaagcaaattccattttcgctgttaggggtctcagcgatgtagctccatgagcggttgctcagcagtgattcacactcgggattccccagaatccagccagcgatgttgcacttgcccagctgcagtggggcgatggctagcacgccctgaggcagcagcaggttgctcaccaccagcagcagcagcagtctgctgcccttctggctgctgcccttgctgtccat

SEQ ID NO: 126 - DNA, 1266 bp, synthetic construct
  misc_feature 1..1266: note = Synthetic
  source 1..1266: mol_type = other DNA
atggacagcaagggcagcagccagaagggcagcagactgctgctgctgctggtggtgagcaacctgctgctgcctcagggcgtgctagccatcgccccactgcagctgggaaaatgcaacatcgctggatggattctgggcaatcccgagtgtgaatcactgctgagcgagcgctcttggagttacatcgtggagacccctaacagcgaaaatgggacatgcttcccaggagactttattgattatgaggaactgcgcgagcagctgagctccgtgtctagtttcgagcgatttgaaatcttctctaaggaatcaagctggccaaaacacaccacaggcggggtgactgccgcttgtagtcatgccggcaagtcctctttctaccggaacctgctgtggctgaccgagaaagacgggtcctaccccaacctgaacaactcttacgtgaataagaagggcaaggaagtgctggtcctgtggggggtccaccatcctagcaacatcaaggatcagcagacactgtaccagaaagagaatgcctatgtgtccgtggtcagttcaaactacaatcggcggttcacccccgagatcgctgaaaggcctaaggtccgcggacaggcaggccgaattaactactattggactctgctgaaacccggggacaccatcatgttcgaggcaaacggaaatctgattgccccttggtatgcttttgcactgtctcgcggggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 127 - protein, 421 aa, synthetic construct
  REGION 1..421: note = Synthetic
MDSKGSSQKGSRLLLLLVVSNLLLPQGVLAIAPLQLGKCNIAGWILGNPECESLLSERSWSYIVETPNSENGTCFPGDFIDYEELREQLSSVSSFERFEIFSKESSWPKHTTGGVTAACSHAGKSSFYRNLLWLTEKDGSYPNLNNSYVNKKGKEVLVLWGVHHPSNIKDQQTLYQKENAYVSVVSSNYNRRFTPEIAERPKVRGQAGRINYYWTLLKPGDTIMFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 128 - DNA, 1266 bp, synthetic construct
  misc_feature 1..1266: note = Synthetic
  source 1..1266: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggccccgcgagacagtgcaaaagcataccaaggggcaatcagatttccgtttgcctcgaacatgatggtgtccccgggtttcagcagagtccaatagtagttaattcggcctgcctgtccgcggaccttaggcctttcagcgatctcgggggtgaaccgccgattgtagtttgaactgaccacggacacataggcattctctttctggtacagtgtctgctgatccttgatgttgctaggatggtggaccccccacaggaccagcacttccttgcccttcttattcacgtaagagttgttcaggttggggtaggacccgtctttctcggtcagccacagcaggttccggtagaaagaggacttgccggcatgactacaagcggcagtcaccccgcctgtggtgtgttttggccagcttgattccttagagaagatttcaaatcgctcgaaactagacacggagctcagctgctcgcgcagttcctcataatcaataaagtctcctgggaagcatgtcccattttcgctgttaggggtctccacgatgtaactccaagagcgctcgctcagcagtgattcacactcgggattgcccagaatccatccagcgatgttgcattttcccagctgcagtggggcgatggctagcacgccctgaggcagcagcaggttgctcaccaccagcagcagcagcagtctgctgcccttctggctgctgcccttgctgtccat

SEQ ID NO: 129 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgcccctctgcagctgggcaagtgctctatcgctggctggattctggggaatccagagtgtgaaagtctgttttcaaagaaatcttggagttacattgctgagacccccaacagcgaaaatggaacatgcttccctggctatttcgcagattatgaggaactgagggagcagctgagctccgtgtctagtttcgagagatttgaaatcttccccaaagaaaggagctggcctaagcacaacgtgacccggggagtcacagcctcatgtagccataagggcaaatcaagcttttacagaaatctgctgtggctgacagagaaaaacgggtcctacccaaatctgtccaagtcttatgtgaacaataaggagaaagaagtgctggtcctgtggggcgtccaccatcccagcaacatcgaggaccagaagactatttaccgaaaagaaaatgcctatgtgtccgtggtctcctctaactacaatcggagatttaccccagagatcgctgaaaggccaaaggtgcgaggacaggcaggacgaattaactactattggactctgctggagccaggggataccatcattttcgaagccaacggaaatctgatcgctccctggtatgcatttgccctgagtcggggagcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 130 - protein, 422 aa, synthetic construct
  REGION 1..422: note = Synthetic
MDSKGSSQKGSRLLLLLVVSNLLLPQGVLAIAPLQLGKCSIAGWILGNPECESLFSKKSWSYIAETPNSENGTCFPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 131 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggctccccgactcagggcaaatgcataccagggagcgatcagatttccgttggcttcgaaaatgatggtatcccctggctccagcagagtccaatagtagttaattcgtcctgcctgtcctcgcacctttggcctttcagcgatctctggggtaaatctccgattgtagttagaggagaccacggacacataggcattttcttttcggtaaatagtcttctggtcctcgatgttgctgggatggtggacgccccacaggaccagcacttctttctccttattgttcacataagacttggacagatttgggtaggacccgtttttctctgtcagccacagcagatttctgtaaaagcttgatttgcccttatggctacatgaggctgtgactccccgggtcacgttgtgcttaggccagctcctttctttggggaagatttcaaatctctcgaaactagacacggagctcagctgctccctcagttcctcataatctgcgaaatagccagggaagcatgttccattttcgctgttgggggtctcagcaatgtaactccaagatttctttgaaaacagactttcacactctggattccccagaatccagccagcgatagagcacttgcccagctgcagaggggcgatggctagcacgccctgaggcagcagcaggttgctcaccaccagcagcagcagcagtctgctgcccttctggctgctgcccttgctgtccat

SEQ ID NO: 132 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgctcctctgcagctgggaaagtgcaacatcgcaggatggattctgggcaatccagagtgtgaatccctgctgtctaaacggtcttggagttacattgccgagacacccaactctgaaaatggggcctgcttccctggagactttgctgattatgaggaactgagagagcagctgagctccgtgtctagtttcgagagatttgaaatcttccccaaggaaaggtcctggcctaaacacaacattactaggggggtgaccgccgcttgttctcatgccggaaaatcaagcttctacaagaatctgctgtggctgacagagactgacggctcctacccaaagctgtcaaaaagctatgtgaacaataaggagaaagaagtgctggtcctgtggggcgtccaccatcccagtaacatcgaggatcagaaaactctgtaccgcaaggaaaatgcttatgtgagcgtggtctcctctaactacaatcggagatttaccccagagatcgcagaaaggccaaaggtgcgaggacaggcaggacgaattaactactattggactctgctggagccaggcgacaccatcattttcgaagcaaacgggaatctgatcgccccctggtatgcttttgcactgtcccgcgatgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 133 - protein, 422 aa, synthetic construct
  REGION 1..422: note = Synthetic
MDSKGSSQKGSRLLLLLVVSNLLLPQGVLAIAPLQLGKCNIAGWILGNPECESLLSKRSWSYIAETPNSENGACFPGDFADYEELREQLSSVSSFERFEIFPKERSWPKHNITRGVTAACSHAGKSSFYKNLLWLTETDGSYPKLSKSYVNNKEKEVLVLWGVHHPSNIEDQKTLYRKENAYVSVVSSNYNRRFTPEIAERPKVRGQAGRINYYWTLLEPGDTIIFEANGNLIAPWYAFALSRDASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 134 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcatcgcgggacagtgcaaaagcataccagggggcgatcagattcccgtttgcttcgaaaatgatggtgtcgcctggctccagcagagtccaatagtagttaattcgtcctgcctgtcctcgcacctttggcctttctgcgatctctggggtaaatctccgattgtagttagaggagaccacgctcacataagcattttccttgcggtacagagttttctgatcctcgatgttactgggatggtggacgccccacaggaccagcacttctttctccttattgttcacatagctttttgacagctttgggtaggagccgtcagtctctgtcagccacagcagattcttgtagaagcttgattttccggcatgagaacaagcggcggtcacccccctagtaatgttgtgtttaggccaggacctttccttggggaagatttcaaatctctcgaaactagacacggagctcagctgctctctcagttcctcataatcagcaaagtctccagggaagcaggccccattttcagagttgggtgtctcggcaatgtaactccaagaccgtttagacagcagggattcacactctggattgcccagaatccatcctgcgatgttgcactttcccagctgcagaggagcgatggctagcacgccctgaggcagcagcaggttgctcaccaccagcagcagcagcagtctgctgcccttctggctgctgcccttgctgtccat

SEQ ID NO: 135 - DNA, 1266 bp, synthetic construct
  misc_feature 1..1266: note = Synthetic
  source 1..1266: mol_type = other DNA
atggacagcaagggcagcagccagaagggcagcagactgctgctgctgctggtggtgagcaacctgctgctgcctcagggcgtgctagccattgcccctctgcagctgggaaattgtagcgtggccggctggattctgggcaatcctgagtgcgagctgctgatttccaaagagtcctggtcctacatcgtggagaagcccaaccctgagaatggcacctgcttccctggccacttcgccgattacgaggaactgagagaacagctgtccagcgtgtccagcttcgagagattcgagatcttccccaaagagagcagctggcccaatcatacagtgaccggcgtgagcgcctcttgtagccacaatggcgagagcagcttctacagaaacctgctgtggctgaccggcaagaacggcctgtaccccaacctgagcaagagctacgccaacaacaaagaaaaagaagtgctggtcctctggggagtgcaccaccctcctaacatcggcatccagaaggccctgtaccacaccgagaatgcctacgtgtccgtggtgtccagccactacagcagaaagttcacccccgagatcgccaaaagacccaaagtgcgggaccaggaaggcaggatcaactactactggaccctgctggaacctggcgacaccatcatcttcgaggccaacggcaatctgatcgcccctagatacgcctttgccctgagcagaggcgcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 136 - protein, 421 aa, synthetic construct
  REGION 1..421: note = Synthetic
MDSKGSSQKGSRLLLLLVVSNLLLPQGVLAIAPLQLGNCSVAGWILGNPECELLISKESWSYIVEKPNPENGTCFPGHFADYEELREQLSSVSSFERFEIFPKESSWPNHTVTGVSASCSHNGESSFYRNLLWLTGKNGLYPNLSKSYANNKEKEVLVLWGVHHPPNIGIQKALYHTENAYVSVVSSHYSRKFTPEIAKRPKVRDQEGRINYYWTLLEPGDTIIFEANGNLIAPRYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 137 - DNA, 1266 bp, synthetic construct
  misc_feature 1..1266: note = Synthetic
  source 1..1266: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggcgcctctgctcagggcaaaggcgtatctaggggcgatcagattgccgttggcctcgaagatgatggtgtcgccaggttccagcagggtccagtagtagttgatcctgccttcctggtcccgcactttgggtcttttggcgatctcgggggtgaactttctgctgtagtggctggacaccacggacacgtaggcattctcggtgtggtacagggccttctggatgccgatgttaggagggtggtgcactccccagaggaccagcacttctttttctttgttgttggcgtagctcttgctcaggttggggtacaggccgttcttgccggtcagccacagcaggtttctgtagaagctgctctcgccattgtggctacaagaggcgctcacgccggtcactgtatgattgggccagctgctctctttggggaagatctcgaatctctcgaagctggacacgctggacagctgttctctcagttcctcgtaatcggcgaagtggccagggaagcaggtgccattctcagggttgggcttctccacgatgtaggaccaggactctttggaaatcagcagctcgcactcaggattgcccagaatccagccggccacgctacaatttcccagctgcagaggggcaatggctagcacgccctgaggcagcagcaggttgctcaccaccagcagcagcagcagtctgctgcccttctggctgctgcccttgctgtccat

SEQ ID NO: 138 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgctcctctgcagctggggaagtgcagcatcgcagggtggattctgggaaatccagagtgtgaatccctgttttctaagaaaagctggtcctacattgccgagacacccaactccgaaaatggcacttgtttccctgggtatttcgctgattatgaggaactgcgcgagcagctgagctccgtgtctagtttcgagcgatttgaaatcttccccaaggaatcaagctggcctaaacacaacgtgacccggggggtcacagcctcttgcagtcataagggaaaatgttctttctacagaaatctgctgtggctgacagagaagaacgggagttacccaaatctgtcaaagagctacgtgaacaataaggagaaagaagtgctggtcctgtggggcgtccaccatccctctaacatcgaggaccagaagactatctaccgaaaagaaaacgcatatgtgagcgtggtctcctctcactacaatcggcggttcacccccgagatcgccaagaggcccaaagtgcgcgaccaggaaggccgcattaactactattggaccctgctggagccaggcgatacaatcattttcgaagccaacgggaatctgatcgctccctggtatgcatttgccctgtcaagaggagcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 139 - protein, 422 aa, synthetic construct
  REGION 1..422: note = Synthetic
MDSKGSSQKGSRLLLLLVVSNLLLPQGVLAIAPLQLGKCSIAGWILGNPECESLFSKKSWSYIAETPNSENGTCFPGYFADYEELREQLSSVSSFERFEIFPKESSWPKHNVTRGVTASCSHKGKCSFYRNLLWLTEKNGSYPNLSKSYVNNKEKEVLVLWGVHHPSNIEDQKTIYRKENAYVSVVSSHYNRRFTPEIAKRPKVRDQEGRINYYWTLLEPGDTIIFEANGNLIAPWYAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 140 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggctcctcttgacagggcaaatgcataccagggagcgatcagattcccgttggcttcgaaaatgattgtatcgcctggctccagcagggtccaatagtagttaatgcggccttcctggtcgcgcactttgggcctcttggcgatctcgggggtgaaccgccgattgtagtgagaggagaccacgctcacatatgcgttttcttttcggtagatagtcttctggtcctcgatgttagagggatggtggacgccccacaggaccagcacttctttctccttattgttcacgtagctctttgacagatttgggtaactcccgttcttctctgtcagccacagcagatttctgtagaaagaacattttcccttatgactgcaagaggctgtgaccccccgggtcacgttgtgtttaggccagcttgattccttggggaagatttcaaatcgctcgaaactagacacggagctcagctgctcgcgcagttcctcataatcagcgaaatacccagggaaacaagtgccattttcggagttgggtgtctcggcaatgtaggaccagcttttcttagaaaacagggattcacactctggatttcccagaatccaccctgcgatgctgcacttccccagctgcagaggagcgatggctagcacgccctgaggcagcagcaggttgctcaccaccagcagcagcagcagtctgctgcccttctggctgctgcccttgctgtccat

SEQ ID NO: 141 - DNA, 1269 bp, synthetic construct
  misc_feature 1..1269: note = Synthetic
  source 1..1269: mol_type = other DNA
atggacagcaagggcagcagccagaagggcagcagactgctgctgctgctggtggtgagcaacctgctgctgcctcagggcgtgctagccatcgcccctctgcagctgggcaagtgcaacatcgctggctggattctggggaatccagagtgtgaatctctgtttagtaagaaaagctggtcctacattgctgagacccccaacagcgaaaatggaacatgcttccctggctatttcgcagattatgaggaactgagggagcagctgagctccgtgtctagtttcgagagatttgaaatcttccccaaggaaaggtcttggcctaaacacaacattacccggggcgtgacagccgcttgtagtcataaggggaaatcaagcttttacagaaacctgctgtggctgacagagaagaatggctcatacccaaacctgaacaagagctatgtgaacaataaggagaaagaagtgctggtcctgtggggcgtccaccatccctctaacatcgaggaccagaagactctgtaccgaaaagaaaatgcctatgtgtccgtggtctcctctaactacaatcggcggttcacccccgagatcgctgaaaggccaaaggtgcgcggacaggcaggacgcattaactactattggactctgctggagccaggagataccatcattttcgaagcaaacggcaatctgatcgccccctggcacgcttttgcactgagccggggagcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 142 - protein, 422 aa, synthetic construct
  REGION 1..422: note = Synthetic
MDSKGSSQKGSRLLLLLVVSNLLLPQGVLAIAPLQLGKCNIAGWILGNPECESLFSKKSWSYIAETPNSENGTCFPGYFADYEELREQLSSVSSFERFEIFPKERSWPKHNITRGVTAACSHKGKSSFYRNLLWLTEKNGSYPNLNKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 143 - DNA, 1269 bp, synthetic construct
  misc_feature 1..1269: note = Synthetic
  source 1..1269: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggctccccggctcagtgcaaaagcgtgccagggggcgatcagattgccgtttgcttcgaaaatgatggtatctcctggctccagcagagtccaatagtagttaatgcgtcctgcctgtccgcgcacctttggcctttcagcgatctcgggggtgaaccgccgattgtagttagaggagaccacggacacataggcattttcttttcggtacagagtcttctggtcctcgatgttagagggatggtggacgccccacaggaccagcacttctttctccttattgttcacatagctcttgttcaggtttgggtatgagccattcttctctgtcagccacagcaggtttctgtaaaagcttgatttccccttatgactacaagcggctgtcacgccccgggtaatgttgtgtttaggccaagacctttccttggggaagatttcaaatctctcgaaactagacacggagctcagctgctccctcagttcctcataatctgcgaaatagccagggaagcatgttccattttcgctgttgggggtctcagcaatgtaggaccagcttttcttactaaacagagattcacactctggattccccagaatccagccagcgatgttgcacttgcccagctgcagaggggcgatggctagcacgccctgaggcagcagcaggttgctcaccaccagcagcagcagcagtctgctgcccttctggctgctgcccttgctgtccat

SEQ ID NO: 144 - DNA, 1266 bp, synthetic construct
  misc_feature 1..1266: note = Synthetic
  source 1..1266: mol_type = other DNA
atggacagcaagggcagcagccagaagggcagcagactgctgctgctgctggtggtgagcaacctgctgctgcctcagggcgtgctagccgtgaagcctctgatcctgagagattgtagcgtggctggatggctgctgggcaaccctatgtgcgacgagttcatcaacgtgcccgagtggagctatatcgtggagaaggccaaccccaccaacgatctgtgtttccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgagcagaccagactgtaccagaaccccaccacctatatcagcatcggcaccagcaccctgaatcagagactggtgcccaagatcgccaccagatccaaggtgaacggccagagcggcaggatggaattcttctggaccatcctgaagcccaacgacgccatcaacttcgagagcaacggcaactttatcgcccctgagtacgcctacaagatcgtgaagaagggctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 145 - protein, 421 aa, synthetic construct
  REGION 1..421: note = Synthetic
MDSKGSSQKGSRLLLLLVVSNLLLPQGVLAVKPLILRDCSVAGWLLGNPMCDEFINVPEWSYIVEKANPTNDLCFPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 146 - DNA, 1266 bp, synthetic construct
  misc_feature 1..1266: note = Synthetic
  source 1..1266: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggagcccttcttcacgatcttgtaggcgtactcaggggcgataaagttgccgttgctctcgaagttgatggcgtcgttgggcttcaggatggtccagaagaattccatcctgccgctctggccgttcaccttggatctggtggcgatcttgggcaccagtctctgattcagggtgctggtgccgatgctgatataggtggtggggttctggtacagtctggtctgctcggcggcatcattagggtggtggattccccacaggaccagcagatcttcctggttggtgttgttgtagctcttcttgatggtggggtaggtgctgttcttcttgatcagccacaccacgtttctgaagaagctggggctgcccaggtaaggacaggcgctagacactccgctagaggcttcgtgatcgctccaagaggacttggggatgatctggatcttctcgaagtggttgatccgggacagcaggtgcttcagttcctcgtaatcgttgaagctgccggggaaacacagatcgttggtggggttggccttctccacgatatagctccactcgggcacgttgatgaactcgtcgcacatagggttgcccagcagccatccagccacgctacaatctctcaggatcagaggcttcacggctagcacgccctgaggcagcagcaggttgctcaccaccagcagcagcagcagtctgctgcccttctggctgctgcccttgctgtccat

SEQ ID NO: 147 - DNA, 1263 bp, synthetic construct
  misc_feature 1..1263: note = Synthetic
  source 1..1263: mol_type = other DNA
atggacagtaaggggtcttcacagaaagggtcacggctgctgctgctgctggtcgtcagtaacctgctgctgcctcagggagtgctagccgtgaaacccctgattctgcgcgactgtagcgtggccggatggctgctgggcaaccctatgtgcgatgagtttattaacgtccctgagtggagctacatcgtggagaaggcatccccagccaacgacctgtgcttccccggcaacttcaatgattatgaggaactgaaacacctgctgtctcgaatcaaccatttcgaaaagatccagatcatcccaaagagctcctggagcaatcacgacgcctctagtggagtctcaagcgcttgtccctatctgggccggtcctctttctttagaaacgtggtctggctgatcaagaaaaattctgcctaccctacaattaagagaagttacaacaacactaatcaggaggacctgctggtgctgtggggcgtccaccatcctaacgatgccgctgaacagaccaaactgtaccagaatccaaccacatatatcagtgtggggacttcaaccctgaaccagaggctggtgcccgagattgcaacccgccctaaggtcaatggccagtccgggcggatggaattcttttggacaatcctgaaacccaacgatgctattaatttcgagagcaacgggaattttatcgctcctgaatacgcatataagattgtgaagaaaggctccggagaaagtcaggtgaggcagcagttcagtaaggatatcgagaaactgctgaacgaacaggtgaacaaggagatgcagtctagtaacctgtacatgagtatgtcaagctggtgttatacccactcactggacggagccggcctgttcctgtttgatcacgcagccgaggaatacgaacatgctaagaaactgatcatttttctgaacgagaacaacgtcccagtgcagctgacaagtatctcagcccccgagcataagttcgaaggcctgactcagatctttcagaaagcctacgaacacgagcagcatattagcgagtccatcaacaatattgtggaccacgcaattaagagcaaagatcatgccaccttcaattttctgcagtggtacgtggccgagcagcacgaggaagaggtgctgttcaaggacatcctggataaaatcgaactgattggcaacgagaatcatgggctgtacctggcagaccagtatgtgaagggcattgctaagtcaagaaaaagctgatga

SEQ ID NO: 148 - protein, 419 aa, synthetic construct
  REGION 1..419: note = Synthetic
MDSKGSSQKGSRLLLLLVVSNLLLPQGVLAVKPLILRDCSVAGWLLGNPMCDEFINVPEWSYIVEKASPANDLCFPGNFNDYEELKHLLSRINHFEKIQIIPKSSWSNHDASSGVSSACPYLGRSSFFRNVVWLIKKNSAYPTIKRSYNNTNQEDLLVLWGVHHPNDAAEQTKLYQNPTTYISVGTSTLNQRLVPEIATRPKVNGQSGRMEFFWTILKPNDAINFESNGNFIAPEYAYKIVKKGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 149 - DNA, 1263 bp, synthetic construct
  misc_feature 1..1263: note = Synthetic
  source 1..1263: mol_type = other DNA
tcatcagctttttcttgacttagcaatgcccttcacatactggtctgccaggtacagcccatgattctcgttgccaatcagttcgattttatccaggatgtccttgaacagcacctcttcctcgtgctgctcggccacgtaccactgcagaaaattgaaggtggcatgatctttgctcttaattgcgtggtccacaatattgttgatggactcgctaatatgctgctcgtgttcgtaggctttctgaaagatctgagtcaggccttcgaacttatgctcgggggctgagatacttgtcagctgcactgggacgttgttctcgttcagaaaaatgatcagtttcttagcatgttcgtattcctcggctgcgtgatcaaacaggaacaggccggctccgtccagtgagtgggtataacaccagcttgacatactcatgtacaggttactagactgcatctccttgttcacctgttcgttcagcagtttctcgatatccttactgaactgctgcctcacctgactttctccggagcctttcttcacaatcttatatgcgtattcaggagcgataaaattcccgttgctctcgaaattaatagcatcgttgggtttcaggattgtccaaaagaattccatccgcccggactggccattgaccttagggcgggttgcaatctcgggcaccagcctctggttcagggttgaagtccccacactgatatatgtggttggattctggtacagtttggtctgttcagcggcatcgttaggatggtggacgccccacagcaccagcaggtcctcctgattagtgttgttgtaacttctcttaattgtagggtaggcagaatttttcttgatcagccagaccacgtttctaaagaaagaggaccggcccagatagggacaagcgcttgagactccactagaggcgtcgtgattgctccaggagctctttgggatgatctggatcttttcgaaatggttgattcgagacagcaggtgtttcagttcctcataatcattgaagttgccggggaagcacaggtcgttggctggggatgccttctccacgatgtagctccactcagggacgttaataaactcatcgcacatagggttgcccagcagccatccggccacgctacagtcgcgcagaatcaggggtttcacggctagcactccctgaggcagcagcaggttactgacgaccagcagcagcagcagccgtgaccctttctgtgaagaccccttactgtccat

SEQ ID NO: 150 - DNA, 1263 bp, synthetic construct
  misc_feature 1..1263: note = Synthetic
  source 1..1263: mol_type = other DNA
atggacagtaaggggtcttcacagaaagggtcacggctgctgctgctgctggtcgtcagtaacctgctgctgcctcagggagtgctagccgtgaagcctctgattctgaaagactgctccgtcgctggatggctgctggggaaccctatgtgtgatgagtttattaacgtgcctgagtggagctacatcgtggagaaggccaaccccgctaatgacctgtgcttccctggcaacttcaatgattatgaggaactgaaacacctgctgagccgaatcaaccattttgagaagattcagatcattcccaaagactcatggagcgatcacgaagcttccctgggagtgagctccgcatgtccttatcagggcaactctagtttctttagaaatgtggtctggctgatcaagaaaggcaacgcctacccaacaattaagaaatcttacaacaacactaatcaggaagacctgctggtcctgtggggcatccaccatccaaacgatgaggccgaacagaccaggctgtaccagaatcccaccacatatatctccattggcacttctaccctgaaccagcggctggtgcccaagatcgccaccagaagtaaagtcaatggccagtcagggcgcatcgacttcttttggacaattctgaagcctaacgatgctattaatttcgagtccaacgggaattttatcgcaccagaatacgcctataagattgtgaagaaaggctccggagaaagtcaggtgaggcagcagttcagtaaggatatcgagaaactgctgaacgaacaggtgaacaaggagatgcagtctagtaacctgtacatgagtatgtcaagctggtgttatacccactcactggacggagccggcctgttcctgtttgatcacgcagccgaggaatacgaacatgctaagaaactgatcatttttctgaacgagaacaacgtcccagtgcagctgacaagtatctcagcccccgagcataagttcgaaggcctgactcagatctttcagaaagcctacgaacacgagcagcatattagcgagtccatcaacaatattgtggaccacgcaattaagagcaaagatcatgccaccttcaattttctgcagtggtacgtggccgagcagcacgaggaagaggtgctgttcaaggacatcctggataaaatcgaactgattggcaacgagaatcatgggctgtacctggcagaccagtatgtgaagggcattgctaagtcaagaaaaagctgatga

SEQ ID NO: 151 - protein, 419 aa, synthetic construct
  REGION 1..419: note = Synthetic
MDSKGSSQKGSRLLLLLVVSNLLLPQGVLAVKPLILKDCSVAGWLLGNPMCDEFINVPEWSYIVEKANPANDLCFPGNFNDYEELKHLLSRINHFEKIQIIPKDSWSDHEASLGVSSACPYQGNSSFFRNVVWLIKKGNAYPTIKKSYNNTNQEDLLVLWGIHHPNDEAEQTRLYQNPTTYISIGTSTLNQRLVPKIATRSKVNGQSGRIDFFWTILKPNDAINFESNGNFIAPEYAYKIVKKGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 152 - DNA, 1263 bp, synthetic construct
  misc_feature 1..1263: note = Synthetic
  source 1..1263: mol_type = other DNA
tcatcagctttttcttgacttagcaatgcccttcacatactggtctgccaggtacagcccatgattctcgttgccaatcagttcgattttatccaggatgtccttgaacagcacctcttcctcgtgctgctcggccacgtaccactgcagaaaattgaaggtggcatgatctttgctcttaattgcgtggtccacaatattgttgatggactcgctaatatgctgctcgtgttcgtaggctttctgaaagatctgagtcaggccttcgaacttatgctcgggggctgagatacttgtcagctgcactgggacgttgttctcgttcagaaaaatgatcagtttcttagcatgttcgtattcctcggctgcgtgatcaaacaggaacaggccggctccgtccagtgagtgggtataacaccagcttgacatactcatgtacaggttactagactgcatctccttgttcacctgttcgttcagcagtttctcgatatccttactgaactgctgcctcacctgactttctccggagcctttcttcacaatcttataggcgtattctggtgcgataaaattcccgttggactcgaaattaatagcatcgttaggcttcagaattgtccaaaagaagtcgatgcgccctgactggccattgactttacttctggtggcgatcttgggcaccagccgctggttcagggtagaagtgccaatggagatatatgtggtgggattctggtacagcctggtctgttcggcctcatcgtttggatggtggatgccccacaggaccagcaggtcttcctgattagtgttgttgtaagatttcttaattgttgggtaggcgttgcctttcttgatcagccagaccacatttctaaagaaactagagttgccctgataaggacatgcggagctcactcccagggaagcttcgtgatcgctccatgagtctttgggaatgatctgaatcttctcaaaatggttgattcggctcagcaggtgtttcagttcctcataatcattgaagttgccagggaagcacaggtcattagcggggttggccttctccacgatgtagctccactcaggcacgttaataaactcatcacacatagggttccccagcagccatccagcgacggagcagtctttcagaatcagaggcttcacggctagcactccctgaggcagcagcaggttactgacgaccagcagcagcagcagccgtgaccctttctgtgaagaccccttactgtccat

SEQ ID NO: 153 - DNA, 1260 bp, synthetic construct
  misc_feature 1..1260: note = Synthetic
  source 1..1260: mol_type = other DNA
atggacagtaaggggtcttcacagaaagggtcacggctgctgctgctgctggtcgtcagtaacctgctgctgcctcagggagtgctagccgtgaaacccctgattctgaaagactgctctgtggctggatggctgctgggcaaccctatgtgcgatgagttcctgaatgtgtccgaatggtcttacatcgtggagaaggccagtccagctaacggactgtgcttccccggcgacttcaatgattatgaggaactgaagcacctgctgtctaggatcaaccatttcgagaagatcaagatcatcccaaagagctcctggtccaatcacgaagcttctggggtgtctagtgcatgtagttatctgggaaagccctcattctttcgcaacctggtctggctgatcaagaaaaacaatacttacccccccatcaaggtcaattatactaacaccaatcaggaagacctgctggtcctgtggggcatccaccatcctaacgatgagacagaacaggtgaagatctaccagaatccaaccacatatatttcagtcggcacaagcactctgaaccagcggctggtgcctaagattgccaccagaagcaaagtcaatggccagtccgggcgaatggagttcttttggacaatcctgaagcccaacgacgctattaatttcgatagcaacggcaacttcatcgcacctgaatacgcctataaaattgtgaagaaagggtccggagaaagtcaggtgaggcagcagttcagtaaggatatcgagaaactgctgaacgaacaggtgaacaaggagatgcagtctagtaacctgtacatgagtatgtcaagctggtgttatacccactcactggacggagccggcctgttcctgtttgatcacgcagccgaggaatacgaacatgctaagaaactgatcatttttctgaacgagaacaacgtcccagtgcagctgacaagtatctcagcccccgagcataagttcgaaggcctgactcagatctttcagaaagcctacgaacacgagcagcatattagcgagtccatcaacaatattgtggaccacgcaattaagagcaaagatcatgccaccttcaattttctgcagtggtacgtggccgagcagcacgaggaagaggtgctgttcaaggacatcctggataaaatcgaactgattggcaacgagaatcatgggctgtacctggcagaccagtatgtgaagggcattgctaagtcaagaaaaagctgatga

SEQ ID NO: 154 - protein, 418 aa, synthetic construct
  REGION 1..418: note = Synthetic
MDSKGSSQKGSRLLLLLVVSNLLLPQGVLAVKPLILKDCSVAGWLLGNPMCDEFLNVSEWSYIVEKASPANGLCFPGDFNDYEELKHLLSRINHFEKIKIIPKSSWSNHEASGVSSACSYLGKPSFFRNLVWLIKKNNTYPPIKVNYTNTNQEDLLVLWGIHHPNDETEQVKIYQNPTTYISVGTSTLNQRLVPKIATRSKVNGQSGRMEFFWTILKPNDAINFDSNGNFIAPEYAYKIVKKGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 155 - DNA, 1260 bp, synthetic construct
  misc_feature 1..1260: note = Synthetic
  source 1..1260: mol_type = other DNA
tcatcagctttttcttgacttagcaatgcccttcacatactggtctgccaggtacagcccatgattctcgttgccaatcagttcgattttatccaggatgtccttgaacagcacctcttcctcgtgctgctcggccacgtaccactgcagaaaattgaaggtggcatgatctttgctcttaattgcgtggtccacaatattgttgatggactcgctaatatgctgctcgtgttcgtaggctttctgaaagatctgagtcaggccttcgaacttatgctcgggggctgagatacttgtcagctgcactgggacgttgttctcgttcagaaaaatgatcagtttcttagcatgttcgtattcctcggctgcgtgatcaaacaggaacaggccggctccgtccagtgagtgggtataacaccagcttgacatactcatgtacaggttactagactgcatctccttgttcacctgttcgttcagcagtttctcgatatccttactgaactgctgcctcacctgactttctccggaccctttcttcacaattttataggcgtattcaggtgcgatgaagttgccgttgctatcgaaattaatagcgtcgttgggcttcaggattgtccaaaagaactccattcgcccggactggccattgactttgcttctggtggcaatcttaggcaccagccgctggttcagagtgcttgtgccgactgaaatatatgtggttggattctggtagatcttcacctgttctgtctcatcgttaggatggtggatgccccacaggaccagcaggtcttcctgattggtgttagtataattgaccttgatgggggggtaagtattgtttttcttgatcagccagaccaggttgcgaaagaatgagggctttcccagataactacatgcactagacaccccagaagcttcgtgattggaccaggagctctttgggatgatcttgatcttctcgaaatggttgatcctagacagcaggtgcttcagttcctcataatcattgaagtcgccggggaagcacagtccgttagctggactggccttctccacgatgtaagaccattcggacacattcaggaactcatcgcacatagggttgcccagcagccatccagccacagagcagtctttcagaatcaggggtttcacggctagcactccctgaggcagcagcaggttactgacgaccagcagcagcagcagccgtgaccctttctgtgaagaccccttactgtccat

SEQ ID NO: 156 - DNA, 1263 bp, synthetic construct
  misc_feature 1..1263: note = Synthetic
  source 1..1263: mol_type = other DNA
atggacagtaaggggtcttcacagaaagggtcacggctgctgctgctgctggtcgtcagtaacctgctgctgcctcagggagtgctagccgtcaaacccctgattctgagagattgtagtgtcgctggctggctgctgggcaaccctatgtgtgatgagtttattaacgtccctgaatggtcttacatcgtggagaaagcaagtcccgccaacgacctgtgcttccctggcgacttcaatgattatgaggaactgaaacacctgctgtcccgaatcaaccattttgagaagattcagatcattcccaaaagctcctggtctaatcacgaagcctctagtggagtctcaagcgcttgtccttatctgggcaagtcctctttctttaggaacgtggtctggctgatcaagaaaaattcaacatacccaaccatcaagcgcagttataacaatactaaccaggaggacctgctggtgctgtggggcatccaccatccaaacgatgccgctgaacagacaaagctgtaccagaatcccaccacatatatcagtgtcgggacttcaaccctgaaccagcggctggtgcccaagattgccaccagaagcaaagtcaatggccagtccgggagaatggaattcttttggacaatcctgaagcctaacgatgccattaatttcgagagcaacgggaattttatcgctccagaatacgcatataaaattgtgaagaaaggctccggagaaagtcaggtgaggcagcagttcagtaaggatatcgagaaactgctgaacgaacaggtgaacaaggagatgcagtctagtaacctgtacatgagtatgtcaagctggtgttatacccactcactggacggagccggcctgttcctgtttgatcacgcagccgaggaatacgaacatgctaagaaactgatcatttttctgaacgagaacaacgtcccagtgcagctgacaagtatctcagcccccgagcataagttcgaaggcctgactcagatctttcagaaagcctacgaacacgagcagcatattagcgagtccatcaacaatattgtggaccacgcaattaagagcaaagatcatgccaccttcaattttctgcagtggtacgtggccgagcagcacgaggaagaggtgctgttcaaggacatcctggataaaatcgaactgattggcaacgagaatcatgggctgtacctggcagaccagtatgtgaagggcattgctaagtcaagaaaaagctgatga

SEQ ID NO: 157 - protein, 419 aa, synthetic construct
  REGION 1..419: note = Synthetic
MDSKGSSQKGSRLLLLLVVSNLLLPQGVLAVKPLILRDCSVAGWLLGNPMCDEFINVPEWSYIVEKASPANDLCFPGDFNDYEELKHLLSRINHFEKIQIIPKSSWSNHEASSGVSSACPYLGKSSFFRNVVWLIKKNSTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 158 - DNA, 1263 bp, synthetic construct
  misc_feature 1..1263: note = Synthetic
  source 1..1263: mol_type = other DNA
tcatcagctttttcttgacttagcaatgcccttcacatactggtctgccaggtacagcccatgattctcgttgccaatcagttcgattttatccaggatgtccttgaacagcacctcttcctcgtgctgctcggccacgtaccactgcagaaaattgaaggtggcatgatctttgctcttaattgcgtggtccacaatattgttgatggactcgctaatatgctgctcgtgttcgtaggctttctgaaagatctgagtcaggccttcgaacttatgctcgggggctgagatacttgtcagctgcactgggacgttgttctcgttcagaaaaatgatcagtttcttagcatgttcgtattcctcggctgcgtgatcaaacaggaacaggccggctccgtccagtgagtgggtataacaccagcttgacatactcatgtacaggttactagactgcatctccttgttcacctgttcgttcagcagtttctcgatatccttactgaactgctgcctcacctgactttctccggagcctttcttcacaattttatatgcgtattctggagcgataaaattcccgttgctctcgaaattaatggcatcgttaggcttcaggattgtccaaaagaattccattctcccggactggccattgactttgcttctggtggcaatcttgggcaccagccgctggttcagggttgaagtcccgacactgatatatgtggtgggattctggtacagctttgtctgttcagcggcatcgtttggatggtggatgccccacagcaccagcaggtcctcctggttagtattgttataactgcgcttgatggttgggtatgttgaatttttcttgatcagccagaccacgttcctaaagaaagaggacttgcccagataaggacaagcgcttgagactccactagaggcttcgtgattagaccaggagcttttgggaatgatctgaatcttctcaaaatggttgattcgggacagcaggtgtttcagttcctcataatcattgaagtcgccagggaagcacaggtcgttggcgggacttgctttctccacgatgtaagaccattcagggacgttaataaactcatcacacatagggttgcccagcagccagccagcgacactacaatctctcagaatcaggggtttgacggctagcactccctgaggcagcagcaggttactgacgaccagcagcagcagcagccgtgaccctttctgtgaagaccccttactgtccat

SEQ ID NO: 159 - DNA, 1263 bp, synthetic construct
  misc_feature 1..1263: note = Synthetic
  source 1..1263: mol_type = other DNA
atggacagtaaggggtcttcacagaaagggtcacggctgctgctgctgctggtcgtcagtaacctgctgctgcctcagggagtgctagccgtcaaacctctgattctgagagactgttccgtggctggatggctgctgggcaaccctatgtgcgatgagtttatcaatgtccccgagtggtcttacatcgtggagaaagcaagtcccgccaacggactgtgcttccctggcgacttcaatgattatgaggaactgaaacacctgctgtcccggatcaaccattttgagaagattcagatcattcccaaaagctcctggtctaatcacgaagcctctagtggggtctcaagcgcttgtccttatcagggaaagtcctctttctttaggaacgtggtctggctgatcaagaaaaattcaacatacccaaccatcaagcgcagttataacaatactaaccaggaggacctgctggtgctgtggggcatccaccatccaaacgatgccgctgaacagacacgactgtaccagaatcccaccacatatatcagtgtcggcacttcaaccctgaaccagcggctggtgcccaagattgccaccagaagcaaagtcaatggccagtccgggagaatggaattcttttggacaatcctgaagcctaacgacgccattaatttcgagagcaacggcaattttatcgctccagaatacgcatataagattgtgaagaaagggtccggagaaagtcaggtgaggcagcagttcagtaaggatatcgagaaactgctgaacgaacaggtgaacaaggagatgcagtctagtaacctgtacatgagtatgtcaagctggtgttatacccactcactggacggagccggcctgttcctgtttgatcacgcagccgaggaatacgaacatgctaagaaactgatcatttttctgaacgagaacaacgtcccagtgcagctgacaagtatctcagcccccgagcataagttcgaaggcctgactcagatctttcagaaagcctacgaacacgagcagcatattagcgagtccatcaacaatattgtggaccacgcaattaagagcaaagatcatgccaccttcaattttctgcagtggtacgtggccgagcagcacgaggaagaggtgctgttcaaggacatcctggataaaatcgaactgattggcaacgagaatcatgggctgtacctggcagaccagtatgtgaagggcattgctaagtcaagaaaaagctgatga

SEQ ID NO: 160 - protein, 419 aa, synthetic construct
  REGION 1..419: note = Synthetic
MDSKGSSQKGSRLLLLLVVSNLLLPQGVLAVKPLILRDCSVAGWLLGNPMCDEFINVPEWSYIVEKASPANGLCFPGDFNDYEELKHLLSRINHFEKIQIIPKSSWSNHEASSGVSSACPYQGKSSFFRNVVWLIKKNSTYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 161 - DNA, 1263 bp, synthetic construct
  misc_feature 1..1263: note = Synthetic
  source 1..1263: mol_type = other DNA
tcatcagctttttcttgacttagcaatgcccttcacatactggtctgccaggtacagcccatgattctcgttgccaatcagttcgattttatccaggatgtccttgaacagcacctcttcctcgtgctgctcggccacgtaccactgcagaaaattgaaggtggcatgatctttgctcttaattgcgtggtccacaatattgttgatggactcgctaatatgctgctcgtgttcgtaggctttctgaaagatctgagtcaggccttcgaacttatgctcgggggctgagatacttgtcagctgcactgggacgttgttctcgttcagaaaaatgatcagtttcttagcatgttcgtattcctcggctgcgtgatcaaacaggaacaggccggctccgtccagtgagtgggtataacaccagcttgacatactcatgtacaggttactagactgcatctccttgttcacctgttcgttcagcagtttctcgatatccttactgaactgctgcctcacctgactttctccggaccctttcttcacaatcttatatgcgtattctggagcgataaaattgccgttgctctcgaaattaatggcgtcgttaggcttcaggattgtccaaaagaattccattctcccggactggccattgactttgcttctggtggcaatcttgggcaccagccgctggttcagggttgaagtgccgacactgatatatgtggtgggattctggtacagtcgtgtctgttcagcggcatcgtttggatggtggatgccccacagcaccagcaggtcctcctggttagtattgttataactgcgcttgatggttgggtatgttgaatttttcttgatcagccagaccacgttcctaaagaaagaggactttccctgataaggacaagcgcttgagaccccactagaggcttcgtgattagaccaggagcttttgggaatgatctgaatcttctcaaaatggttgatccgggacagcaggtgtttcagttcctcataatcattgaagtcgccagggaagcacagtccgttggcgggacttgctttctccacgatgtaagaccactcggggacattgataaactcatcgcacatagggttgcccagcagccatccagccacggaacagtctctcagaatcagaggtttgacggctagcactccctgaggcagcagcaggttactgacgaccagcagcagcagcagccgtgaccctttctgtgaagaccccttactgtccat

SEQ ID NO: 162 - DNA, 1251 bp, synthetic construct
  misc_feature 1..1251: note = Synthetic
  source 1..1251: mol_type = other DNA
atggacagcaagggcagcagccagaagggcagcagactgctgctgctgctggtggtgagcaacctgctgctgcctcagggcgtgctagcccaccagatcctggatggcgaaaactgtacactgattgacgctctgctgggagaccctcagtgcgatggcttccagaataagaaatgggatctgtttgtggagaggtctaaggcatacagtaactgtttcccctatgacgtgcctgattatgcaagcctgcgctccctggtcgcctctagtggcacactggagttcaacaatgaaagctttaattggacaggggtgactcagaacggaacttcaagcgcctgcatccggagatctaacaacagtttcttttcaagactgaactggctgacccagctgaatttcaagtaccctgctctgaacgtgacaatgccaaacaatgagcagtttgacaagctgtatatctggggcgtgcaccatcccgtcaccgacaaagatcagatcttcctgtacgcacagtcctctggcaggattaccgtgtcaacaaagcgcagccagcaggccgtcatccctaatattgggtacaggccacgcatccgaaacattcccagccgcatctccatctactggactatcgtgaaaccaggcgatatcctgctgattaactccaccggaaatctgattgccccccggggctatttcaagattagaagtggggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 163 - protein, 416 aa, synthetic construct
  REGION 1..416: note = Synthetic
MDSKGSSQKGSRLLLLLVVSNLLLPQGVLAHQILDGENCTLIDALLGDPQCDGFQNKKWDLFVERSKAYSNCFPYDVPDYASLRSLVASSGTLEFNNESFNWTGVTQNGTSSACIRRSNNSFFSRLNWLTQLNFKYPALNVTMPNNEQFDKLYIWGVHHPVTDKDQIFLYAQSSGRITVSTKRSQQAVIPNIGYRPRIRNIPSRISIYWTIVKPGDILLINSTGNLIAPRGYFKIRSGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 164 - DNA, 1251 bp, synthetic construct
  misc_feature 1..1251: note = Synthetic
  source 1..1251: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggccccacttctaatcttgaaatagccccggggggcaatcagatttccggtggagttaatcagcaggatatcgcctggtttcacgatagtccagtagatggagatgcggctgggaatgtttcggatgcgtggcctgtacccaatattagggatgacggcctgctggctgcgctttgttgacacggtaatcctgccagaggactgtgcgtacaggaagatctgatctttgtcggtgacgggatggtgcacgccccagatatacagcttgtcaaactgctcattgtttggcattgtcacgttcagagcagggtacttgaaattcagctgggtcagccagttcagtcttgaaaagaaactgttgttagatctccggatgcaggcgcttgaagttccgttctgagtcacccctgtccaattaaagctttcattgttgaactccagtgtgccactagaggcgaccagggagcgcaggcttgcataatcaggcacgtcataggggaaacagttactgtatgccttagacctctccacaaacagatcccatttcttattctggaagccatcgcactgagggtctcccagcagagcgtcaatcagtgtacagttttcgccatccaggatctggtgggctagcacgccctgaggcagcagcaggttgctcaccaccagcagcagcagcagtctgctgcccttctggctgctgcccttgctgtccat

SEQ ID NO: 165 - DNA, 1320 bp, synthetic construct
  misc_feature 1..1320: note = Synthetic
  source 1..1320: mol_type = other DNA
atggacagcaagggcagcagccagaagggcagcagactgctgctgctgctggtggtgagcaacctgctgctgcctcagggcgtgctagccaaaacacgcggaaaactgtgccccgactgtctgaactgcaccgacctggatgtggcactgggacgacctatgtgtgtcggaaccacaccatccgcaaaagcctctattctgcacgaggtgcggcccgtcacttctggctgcttccctatcatgcatgaccggaccaagattagacagctggccaacctgctgagggggtacgaaaatatccgcctgtccacacagaacgtgattgatgctgagaaggcaccaggaggaccatatcgcctgggaacctccgggtcttgtcccaatgctacaagtaaatcaggcttctttgcaactatggcttgggcagtgcctaaggacaacaacaagaacgctacaaatcccctgactgtggaagtcccttacatctgcgcagagggggaagaccagattaccgtgtggggatttcactctgacgataagacacagatgaaaaacctgtacggggatagtaatcctcagaagttcaccagctccgccaacggagtgactacccattatgtcagtcagatcggaggcttcccagaccagactgaggatgggggactgccccagtcaggcagaatcgtggtcgactacatgatgcagaagcctggaaaaactggcaccattgtgtatcagagaggagtcctgctgccacagaaagtgtggtgtgcatcaggcaggagctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 166 - protein, 439 aa, synthetic construct
  REGION 1..439: note = Synthetic
MDSKGSSQKGSRLLLLLVVSNLLLPQGVLAKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 167 - DNA, 1320 bp, synthetic construct
  misc_feature 1..1320: note = Synthetic
  source 1..1320: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggagctcctgcctgatgcacaccacactttctgtggcagcaggactcctctctgatacacaatggtgccagtttttccaggcttctgcatcatgtagtcgaccacgattctgcctgactggggcagtcccccatcctcagtctggtctgggaagcctccgatctgactgacataatgggtagtcactccgttggcggagctggtgaacttctgaggattactatccccgtacaggtttttcatctgtgtcttatcgtcagagtgaaatccccacacggtaatctggtcttccccctctgcgcagatgtaagggacttccacagtcaggggatttgtagcgttcttgttgttgtccttaggcactgcccaagccatagttgcaaagaagcctgatttacttgtagcattgggacaagacccggaggttcccaggcgatatggtcctcctggtgccttctcagcatcaatcacgttctgtgtggacaggcggatattttcgtaccccctcagcaggttggccagctgtctaatcttggtccggtcatgcatgatagggaagcagccagaagtgacgggccgcacctcgtgcagaatagaggcttttgcggatggtgtggttccgacacacataggtcgtcccagtgccacatccaggtcggtgcagttcagacagtcggggcacagttttccgcgtgttttggctagcacgccctgaggcagcagcaggttgctcaccaccagcagcagcagcagtctgctgcccttctggctgctgcccttgctgtccat

SEQ ID NO: 168 - DNA, 1323 bp, synthetic construct
  misc_feature 1..1323: note = Synthetic
  source 1..1323: mol_type = other DNA
atggacagcaagggcagcagccagaagggcagcagactgctgctgctgctggtggtgagcaacctgctgctgcctcagggcgtgctagccgagacaagaggcaagctgtgccccaagtgcctgaactgtaccgatctggatgtggccctgggcagacctaagtgtaccggcaagatccctagcgccagagtgtctatcctgcacgaagtgcggcctgtgaccagcggctgcttccccatcatgcacgaccggaccaagatcagacagctgcccaatctgctgagaggctacgagcacatcagactgagcacccacaacgtgatcaatgccgagaatgcccctggcggcccttacaagatcggcacctccggcagctgtcccaacatcaccaacggcaacggcttttttgccacaatggcctgggccgtgcctaagaacgacaagaacaagaccgccaccaaccctctgacaatcgaggtgccctacatctgtaccgagggcgaggatcagatcacagtgtggggcttccacagcgacaacgagactcagatggccaagctgtacggcgacagcaagccccagaagtttaccagcagcgccaatggcgtgaccacccactacgtgtctcagatcggcggcttccctaatcagacagaggatggcggcctgcctcagagcggcagaatcgtggtggactacatggtgcagaagtccggcaagaccggcaccatcacctaccagagaggcatcctgctgcctcagaaagtgtggtgtgccagcggcagatcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 169 - protein, 440 aa, synthetic construct
  REGION 1..440: note = Synthetic
MDSKGSSQKGSRLLLLLVVSNLLLPQGVLAETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 170 - DNA, 1323 bp, synthetic construct
  misc_feature 1..1323: note = Synthetic
  source 1..1323: mol_type = other DNA
ctaggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaggatctgccgctggcacaccacactttctgaggcagcaggatgcctctctggtaggtgatggtgccggtcttgccggacttctgcaccatgtagtccaccacgattctgccgctctgaggcaggccgccatcctctgtctgattagggaagccgccgatctgagacacgtagtgggtggtcacgccattggcgctgctggtaaacttctggggcttgctgtcgccgtacagcttggccatctgagtctcgttgtcgctgtggaagccccacactgtgatctgatcctcgccctcggtacagatgtagggcacctcgattgtcagagggttggtggcggtcttgttcttgtcgttcttaggcacggcccaggccattgtggcaaaaaagccgttgccgttggtgatgttgggacagctgccggaggtgccgatcttgtaagggccgccaggggcattctcggcattgatcacgttgtgggtgctcagtctgatgtgctcgtagcctctcagcagattgggcagctgtctgatcttggtccggtcgtgcatgatggggaagcagccgctggtcacaggccgcacttcgtgcaggatagacactctggcgctagggatcttgccggtacacttaggtctgcccagggccacatccagatcggtacagttcaggcacttggggcacagcttgcctcttgtctcggctagcacgccctgaggcagcagcaggttgctcaccaccagcagcagcagcagtctgctgcccttctggctgctgcccttgctgtccat

SEQ ID NO: 171 - DNA, 2604 bp, synthetic construct
  misc_feature 1..2604: note = Synthetic
  source 1..2604: mol_type = other DNA
atggacagcaagggcagcagccagaagggctccagactgctgctgctcctggtggtgtccaacctcctgctgcctcagggcgtgctagccgtggctcctctgcacctgggcaagtgcaatatcgccggctggattctgggcaaccccgagtgcgagagcctgagcacagccagcagctggtcctacatcgtggaaacccctagcagcgacaacggcacctgtttccccggcgacttcatcgactacgaggaactgcgcgagcagctgagcagcgtgtccagcttcgagagattcgagatcttccccaagacctccagctggcccaaccacgacagcaacaaaggcgtgaccgccgcctgtcctcacgctggcgccaagagcttctacaagaacctgatctggctggtcaagaagggcaacagctaccccaagctgagcaagagctacatcaacgacaagggcaaagaggtgctggtcctctggggcatccaccaccctagcacaagcgccgaccagcagagcctgtaccagaacgccgacgcctacgtgttcgtgggcagctcccggtacagcaagaagttcaagcccgagatcgccatccggcccaaagtgcgggaccaggaaggccggatgaactactactggaccctggtggaacccggcgacaagatcaccttcgaggccaccggcaatctggtggtgcccagatacgccttcgccatggaaagaaacgccagcggcgagagccaagtccgacagcagttcagcaaggacatcgagaagctgctgaacgagcaggtcaacaaagagatgcagagcagcaacctgtacatgagcatgtccagctggtgttacacccacagcctggacggcgctggcctgttcctgtttgatcacgccgccgaggaatacgagcacgccaagaagctgatcatcttcctgaacgagaacaacgtgcccgtgcagctgaccagcatcagcgccccagagcacaagttcgagggcctgacccagatcttccagaaggcctacgaacacgagcagcacatcagcgagagcatcaacaatatcgtggaccacgccatcaagagcaaggatcacgccaccttcaactttctgcaatggtacgtggccgaacagcacgaggaagaagtgctgtttaaggacatcctggacaagatcgagctgatcggcaacgagaaccacggcctgtacctggccgaccagtacgtgaagggcattgccaagagcagaaagagccggaagcggagatctggcagcggcgctcctgtgaagcagaccctgaacttcgacctgctgaagctggccggcgacgtggaaagcaaccctgggcccatggactccaagggctcctcccagaaaggatctcggctgctcctcctgctcgtggtgtctaatctgctgctgccacagggtgtcctggcccaccagatcctggatggcgagaactgcaccctgatcgacgccctgctgggcgaccctcagtgcgacggcttccagaacaagaagtgggacctgttcgtcgagcggagcaaggcctacagcaactgcttcccctacgacgtgcccgactacgccagcctgagaagcctggtggccagcagcggcaccctggaattcaacaacgagagcttcaactggaacggcgtgacccagaacggcaccagctccgcctgcatcagaagaagcaacaacagcttcttctcccggctgaactggctgacccacctgaatttcaagtaccccgccctgaacgtgaccatgcccaacaatgagcagttcgacaagctgtacatctggggagtgcaccaccccgtgaccgacaaggaccagatctttctgtacgcccagcccagcggccggatcaccgtgtctaccaagagaagccagcaggccgtgatccccaacatcggcttccggcccaggatcagaaacatccccagccggatcagcatctactggacaatcgtgaagcctggcgacatcctgctgatcaacagcaccggcaacctgatcgcccctcggggctacttcaagatcagaagcggcgcctccggagaatctcaagtccgccagcagttttctaaggacatcgaaaagctgctcaatgaacaggtcaacaaagagatgcagtcctctaacctgtatatgagtatgagttcctggtgctatacccactctctcgacggtgcagggctgttcctcttcgaccacgctgcagaggaatatgaacatgctaagaaactgattatctttctcaacgaaaacaacgtgccagtccagctcaccagtatctctgcccctgaacataagtttgaggggctcactcagatctttcagaaagcttacgagcacgagcagcatatctctgagtctattaacaacatcgtcgaccatgctatcaaatctaaagaccacgctacttttaactttctccaatggtacgtcgcagagcagcatgaggaagaggtcctcttcaaggacattctcgacaaaattgaactcatcggaaacgaaaaccatgggctctacctggctgatcagtacgtcaagggaatcgcaaaaagccggaagtcttgatga

SEQ ID NO: 172 - protein, 865 aa, synthetic construct
  REGION 1..865: note = Synthetic
DSKGSSQKGSRLLLLLVVSNLLLPQGVLAVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSRKRRSGSGAPVKQTLNFDLLKLAGDVESNPGPMDSKGSSQKGSRLLLLLVVSNLLLPQGVLAHQILDGENCTLIDALLGDPQCDGFQNKKWDLFVERSKAYSNCFPYDVPDYASLRSLVASSGTLEFNNESFNWNGVTQNGTSSACIRRSNNSFFSRLNWLTHLNFKYPALNVTMPNNEQFDKLYIWGVHHPVTDKDQIFLYAQPSGRITVSTKRSQQAVIPNIGFRPRIRNIPSRISIYWTIVKPGDILLINSTGNLIAPRGYFKIRSGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 173 - DNA, 2604 bp, synthetic construct
  misc_feature 1..2604: note = Synthetic
  source 1..2604: mol_type = other DNA
tcatcaagacttccggctttttgcgattcccttgacgtactgatcagccaggtagagcccatggttttcgtttccgatgagttcaattttgtcgagaatgtccttgaagaggacctcttcctcatgctgctctgcgacgtaccattggagaaagttaaaagtagcgtggtctttagatttgatagcatggtcgacgatgttgttaatagactcagagatatgctgctcgtgctcgtaagctttctgaaagatctgagtgagcccctcaaacttatgttcaggggcagagatactggtgagctggactggcacgttgttttcgttgagaaagataatcagtttcttagcatgttcatattcctctgcagcgtggtcgaagaggaacagccctgcaccgtcgagagagtgggtatagcaccaggaactcatactcatatacaggttagaggactgcatctctttgttgacctgttcattgagcagcttttcgatgtccttagaaaactgctggcggacttgagattctccggaggcgccgcttctgatcttgaagtagccccgaggggcgatcaggttgccggtgctgttgatcagcaggatgtcgccaggcttcacgattgtccagtagatgctgatccggctggggatgtttctgatcctgggccggaagccgatgttggggatcacggcctgctggcttctcttggtagacacggtgatccggccgctgggctgggcgtacagaaagatctggtccttgtcggtcacggggtggtgcactccccagatgtacagcttgtcgaactgctcattgttgggcatggtcacgttcagggcggggtacttgaaattcaggtgggtcagccagttcagccgggagaagaagctgttgttgcttcttctgatgcaggcggagctggtgccgttctgggtcacgccgttccagttgaagctctcgttgttgaattccagggtgccgctgctggccaccaggcttctcaggctggcgtagtcgggcacgtcgtaggggaagcagttgctgtaggccttgctccgctcgacgaacaggtcccacttcttgttctggaagccgtcgcactgagggtcgcccagcagggcgtcgatcagggtgcagttctcgccatccaggatctggtgggccaggacaccctgtggcagcagcagattagacaccacgagcaggaggagcagccgagatcctttctgggaggagcccttggagtccatgggcccagggttgctttccacgtcgccggccagcttcagcaggtcgaagttcagggtctgcttcacaggagcgccgctgccagatctccgcttccggctctttctgctcttggcaatgcccttcacgtactggtcggccaggtacaggccgtggttctcgttgccgatcagctcgatcttgtccaggatgtccttaaacagcacttcttcctcgtgctgttcggccacgtaccattgcagaaagttgaaggtggcgtgatccttgctcttgatggcgtggtccacgatattgttgatgctctcgctgatgtgctgctcgtgttcgtaggccttctggaagatctgggtcaggccctcgaacttgtgctctggggcgctgatgctggtcagctgcacgggcacgttgttctcgttcaggaagatgatcagcttcttggcgtgctcgtattcctcggcggcgtgatcaaacaggaacaggccagcgccgtccaggctgtgggtgtaacaccagctggacatgctcatgtacaggttgctgctctgcatctctttgttgacctgctcgttcagcagcttctcgatgtccttgctgaactgctgtcggacttggctctcgccgctggcgtttctttccatggcgaaggcgtatctgggcaccaccagattgccggtggcctcgaaggtgatcttgtcgccgggttccaccagggtccagtagtagttcatccggccttcctggtcccgcactttgggccggatggcgatctcgggcttgaacttcttgctgtaccgggagctgcccacgaacacgtaggcgtcggcgttctggtacaggctctgctggtcggcgcttgtgctagggtggtggatgccccagaggaccagcacctctttgcccttgtcgttgatgtagctcttgctcagcttggggtagctgttgcccttcttgaccagccagatcaggttcttgtagaagctcttggcgccagcgtgaggacaggcggcggtcacgcctttgttgctgtcgtggttgggccagctggaggtcttggggaagatctcgaatctctcgaagctggacacgctgctcagctgctcgcgcagttcctcgtagtcgatgaagtcgccggggaaacaggtgccgttgtcgctgctaggggtttccacgatgtaggaccagctgctggctgtgctcaggctctcgcactcggggttgcccagaatccagccggcgatattgcacttgcccaggtgcagaggagccacggctagcacgccctgaggcagcaggaggttggacaccaccaggagcagcagcagtctggagcccttctggctgctgcccttgctgtccat

SEQ ID NO: 174 - DNA, 2727 bp, synthetic construct
  misc_feature 1..2727: note = Synthetic
  source 1..2727: mol_type = other DNA
atggacagcaagggcagcagccagaagggctccagactgctgctgctcctggtggtgtccaacctcctgctgcctcagggcgtgctagccgagacaagaggcaagctgtgccccaagtgcctgaactgcaccgacctggatgtggccctgggcagacctaagtgcaccggcaagatccccagcgccagagtgtccatcctgcacgaagtgcggcctgtgaccagcggctgcttccccatcatgcacgaccggaccaagatcagacagctgcccaacctgctgcggggctacgagcacatcagactgagcacccacaacgtgatcaacgccgagaatgcccctggcggcccttacaagatcggcaccagcggaagctgccccaacatcaccaacggcaacggctttttcgccaccatggcctgggccgtgcccaagaacgacaagaacaagaccgccaccaaccctctgaccatcgaggtgccctacatctgcaccgagggcgaggaccagatcaccgtgtggggcttccacagcgacgacgagactcagatggccaagctgtacggcgacagcaagccccagaagttcaccagcagcgccaacggcgtgaccacccactatgtgtcccagatcggcggcttccctaaccagacagaggatggcggcctgccccagagcggcagaatcgtggtggactacatggtgcagaagtccggcaagaccggcacaatcacctaccagagaggcatcctgctgcctcagaaagtgtggtgcgccagcggcagatctagcggcgaatctcaagtccgacagcagttcagcaaggacatcgagaagctgctgaacgagcaggtcaacaaagagatgcagagcagcaacctgtacatgagcatgagcagctggtgctacacccacagcctggacggcgctggcctgttcctgtttgatcacgccgccgaggaatacgagcacgccaagaagctgatcatcttcctgaacgagaacaacgtgcccgtgcagctgaccagcatcagcgcccctgagcacaagttcgagggcctgacccagatcttccagaaggcctacgaacacgagcagcacatctccgagagcatcaacaacatcgtggaccacgccatcaagagcaaggatcacgccaccttcaactttctgcagtggtacgtggccgaacagcacgaggaagaggtgctgtttaaggacatcctggacaagatcgagctgatcggcaacgagaaccacggcctgtacctggccgaccagtacgtgaagggaatcgccaagagcagaaagagccggaagcggagatctggcagcggcgctcctgtgaagcagaccctgaacttcgacctgctgaagctggccggcgacgtggaatctaatcctgggcccatggatagcaaaggctctagccagaaaggcagccgactcctgctcctgctggtcgtcagtaacctgctgctgccccagggcgtcctcgccaagacaagaggcaagctgtgccccgactgcctgaattgcaccgacctggatgtggccctgggcagacctatgtgcgtgggcacaacacctagcgccaaggccagcatcctgcacgaagtgcggcctgtgaccagcggctgcttccctatcatgcacgaccggaccaagatcaggcagctggccaatctgctgagaggctacgagaacatccggctgagcacccagaatgtgatcgatgccgagaaggcccctggcggcccttacagactgggcacaagcggcagctgtcccaacgccaccagcaagagcggctttttcgccacaatggcctgggccgtgcccaaggacaacaacaagaatgccaccaaccctctgaccgtggaagtgccctacatctgcgccgagggcgaggatcagatcacagtgtggggcttccacagcgacgacaagacccagatgaagaacctgtacggcgacagcaatccccagaagttcacctccagcgccaatggcgtgaccacccactacgtgtcccagatcggcggcttccccgatcagacagaggatggcggactgccccagtccggcagaatcgtggtggactacatgatgcagaagcccggcaagaccggcaccatcgtgtaccagagaggcgtgctgctccctcagaaagtgtggtgcgcctctggcagaagctccggagaatctcaagtccgccagcagttttctaaggacatcgaaaagctgctcaatgaacaggtcaacaaagagatgcagtcctctaacctgtatatgagtatgagttcctggtgctatacccactctctcgacggtgcagggctgttcctcttcgaccacgctgcagaggaatatgaacatgctaagaaactgattatctttctcaacgaaaacaacgtgccagtccagctcaccagtatctctgcccctgaacataagtttgaggggctcactcagatctttcagaaagcttacgagcacgagcagcatatctctgagtctattaacaacatcgtcgaccatgctatcaaatctaaagaccacgctacttttaactttctccaatggtacgtcgcagagcagcatgaggaagaggtcctcttcaaggacattctcgacaaaattgaactcatcggaaacgaaaaccatgggctctacctggctgatcagtacgtcaagggaatcgcaaaaagccggaagtcttgatga

SEQ ID NO: 175 - protein, 907 aa, synthetic construct
  REGION 1..907: note = Synthetic
MDSKGSSQKGSRLLLLLVVSNLLLPQGVLAETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDDETQMAKLYGDSKPQKFTSSANGVTTHYVSQIGGFPNQTEDGGLPQSGRIVVDYMVQKSGKTGTITYQRGILLPQKVWCASGRSSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSRKRRSGSGAPVKQTLNFDLLKLAGDVESNPGPMDSKGSSQKGSRLLLLLVVSNLLLPQGVLAKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 176 - DNA, 2727 bp, synthetic construct
  misc_feature 1..2727: note = Synthetic
  source 1..2727: mol_type = other DNA
tcatcaagacttccggctttttgcgattcccttgacgtactgatcagccaggtagagcccatggttttcgtttccgatgagttcaattttgtcgagaatgtccttgaagaggacctcttcctcatgctgctctgcgacgtaccattggagaaagttaaaagtagcgtggtctttagatttgatagcatggtcgacgatgttgttaatagactcagagatatgctgctcgtgctcgtaagctttctgaaagatctgagtgagcccctcaaacttatgttcaggggcagagatactggtgagctggactggcacgttgttttcgttgagaaagataatcagtttcttagcatgttcatattcctctgcagcgtggtcgaagaggaacagccctgcaccgtcgagagagtgggtatagcaccaggaactcatactcatatacaggttagaggactgcatctctttgttgacctgttcattgagcagcttttcgatgtccttagaaaactgctggcggacttgagattctccggagcttctgccagaggcgcaccacactttctgagggagcagcacgcctctctggtacacgatggtgccggtcttgccgggcttctgcatcatgtagtccaccacgattctgccggactggggcagtccgccatcctctgtctgatcggggaagccgccgatctgggacacgtagtgggtggtcacgccattggcgctggaggtgaacttctggggattgctgtcgccgtacaggttcttcatctgggtcttgtcgtcgctgtggaagccccacactgtgatctgatcctcgccctcggcgcagatgtagggcacttccacggtcagagggttggtggcattcttgttgttgtccttgggcacggcccaggccattgtggcgaaaaagccgctcttgctggtggcgttgggacagctgccgcttgtgcccagtctgtaagggccgccaggggccttctcggcatcgatcacattctgggtgctcagccggatgttctcgtagcctctcagcagattggccagctgcctgatcttggtccggtcgtgcatgatagggaagcagccgctggtcacaggccgcacttcgtgcaggatgctggccttggcgctaggtgttgtgcccacgcacataggtctgcccagggccacatccaggtcggtgcaattcaggcagtcggggcacagcttgcctcttgtcttggcgaggacgccctggggcagcagcaggttactgacgaccagcaggagcaggagtcggctgcctttctggctagagcctttgctatccatgggcccaggattagattccacgtcgccggccagcttcagcaggtcgaagttcagggtctgcttcacaggagcgccgctgccagatctccgcttccggctctttctgctcttggcgattcccttcacgtactggtcggccaggtacaggccgtggttctcgttgccgatcagctcgatcttgtccaggatgtccttaaacagcacctcttcctcgtgctgttcggccacgtaccactgcagaaagttgaaggtggcgtgatccttgctcttgatggcgtggtccacgatgttgttgatgctctcggagatgtgctgctcgtgttcgtaggccttctggaagatctgggtcaggccctcgaacttgtgctcaggggcgctgatgctggtcagctgcacgggcacgttgttctcgttcaggaagatgatcagcttcttggcgtgctcgtattcctcggcggcgtgatcaaacaggaacaggccagcgccgtccaggctgtgggtgtagcaccagctgctcatgctcatgtacaggttgctgctctgcatctctttgttgacctgctcgttcagcagcttctcgatgtccttgctgaactgctgtcggacttgagattcgccgctagatctgccgctggcgcaccacactttctgaggcagcaggatgcctctctggtaggtgattgtgccggtcttgccggacttctgcaccatgtagtccaccacgattctgccgctctggggcaggccgccatcctctgtctggttagggaagccgccgatctgggacacatagtgggtggtcacgccgttggcgctgctggtgaacttctggggcttgctgtcgccgtacagcttggccatctgagtctcgtcgtcgctgtggaagccccacacggtgatctggtcctcgccctcggtgcagatgtagggcacctcgatggtcagagggttggtggcggtcttgttcttgtcgttcttgggcacggcccaggccatggtggcgaaaaagccgttgccgttggtgatgttggggcagcttccgctggtgccgatcttgtaagggccgccaggggcattctcggcgttgatcacgttgtgggtgctcagtctgatgtgctcgtagccccgcagcaggttgggcagctgtctgatcttggtccggtcgtgcatgatggggaagcagccgctggtcacaggccgcacttcgtgcaggatggacactctggcgctggggatcttgccggtgcacttaggtctgcccagggccacatccaggtcggtgcagttcaggcacttggggcacagcttgcctcttgtctcggctagcacgccctgaggcagcaggaggttggacaccaccaggagcagcagcagtctggagcccttctggctgctgcccttgctgtccat

SEQ ID NO: 177 - DNA, 4011 bp, synthetic construct
  misc_feature 1..4011: note = Synthetic
  source 1..4011: mol_type = other DNA
atggacagcaagggcagcagccagaagggctccagactgctgctgctcctggtggtgtccaacctcctgctgcctcagggcgtgctagccgtggctcctctgcacctgggcaagtgcaatatcgccggctggattctgggcaaccccgagtgcgagagcctgagcacagccagcagctggtcctacatcgtggaaacccctagcagcgacaacggcacctgtttccccggcgacttcatcgactacgaggaactgcgcgagcagctgagcagcgtgtccagcttcgagagattcgagatcttccccaagacctccagctggcccaaccacgacagcaacaaaggcgtgaccgccgcctgtcctcacgctggcgccaagagcttctacaagaacctgatctggctggtcaagaagggcaacagctaccccaagctgagcaagagctacatcaacgacaagggcaaagaggtgctggtcctctggggcatccaccaccctagcacaagcgccgaccagcagagcctgtaccagaacgccgacgcctacgtgttcgtgggcagctcccggtacagcaagaagttcaagcccgagatcgccatccggcccaaagtgcgggaccaggaaggccggatgaactactactggaccctggtggaacccggcgacaagatcaccttcgaggccaccggcaatctggtggtgcccagatacgccttcgccatggaaagaaacgccagcggcgagagccaagtccgacagcagttcagcaaggacatcgagaagctgctgaacgagcaggtcaacaaagagatgcagagcagcaacctgtacatgagcatgtccagctggtgttacacccacagcctggacggcgctggcctgttcctgtttgatcacgccgccgaggaatacgagcacgccaagaagctgatcatcttcctgaacgagaacaacgtgcccgtgcagctgaccagcatcagcgccccagagcacaagttcgagggcctgacccagatcttccagaaggcctacgaacacgagcagcacatcagcgagagcatcaacaatatcgtggaccacgccatcaagagcaaggatcacgccaccttcaactttctgcaatggtacgtggccgaacagcacgaggaagaagtgctgtttaaggacatcctggacaagatcgagctgatcggcaacgagaaccacggcctgtacctggccgaccagtacgtgaagggcattgccaagagcagaaagagccggaagcggagatctggcagcggcgctcctgtgaagcagaccctgaacttcgacctgctgaagctggccggcgacgtggaaagcaaccctgggcccatggactccaagggctcctcccagaaaggatctcggctgctcctcctgctcgtggtgtctaatctgctgctgccacagggtgtcctggcccaccagatcctggatggcgagaactgcaccctgatcgacgccctgctgggcgaccctcagtgcgacggcttccagaacaagaagtgggacctgttcgtcgagcggagcaaggcctacagcaactgcttcccctacgacgtgcccgactacgccagcctgagaagcctggtggccagcagcggcaccctggaattcaacaacgagagcttcaactggaacggcgtgacccagaacggcaccagctccgcctgcatcagaagaagcaacaacagcttcttctcccggctgaactggctgacccacctgaatttcaagtaccccgccctgaacgtgaccatgcccaacaatgagcagttcgacaagctgtacatctggggagtgcaccaccccgtgaccgacaaggaccagatctttctgtacgcccagcccagcggccggatcaccgtgtctaccaagagaagccagcaggccgtgatccccaacatcggcttccggcccaggatcagaaacatccccagccggatcagcatctactggacaatcgtgaagcctggcgacatcctgctgatcaacagcaccggcaacctgatcgcccctcggggctacttcaagatcagaagcggcgcctccggagagtcccaagtgcgccagcagttttccaaggatattgagaaactcctcaatgaacaggtcaacaaggaaatgcagtcctccaacctctacatgtctatgtcctcttggtgctacacacactccctggatggggccggactgtttctgttcgaccatgccgctgaagagtatgaacacgccaaaaaactcatcatttttctcaatgagaacaatgtccctgtccagctcacctccatctccgctcccgagcacaaatttgaaggactcacacagatttttcagaaagcctatgagcatgaacagcacatttccgagtccatcaacaacattgtcgatcatgccattaagtccaaggaccatgctacattcaatttcctccaatggtatgtcgctgagcagcatgaagaggaagtcctgttcaaagatatcctcgataagatcgaactcattgggaatgagaatcacgggctctatctcgccgatcagtatgtgaaagggatcgctaagtcccggaagtccagaaagcggagaagcggctctggcgccccagtcaaacagacactgaattttgatctgctcaagctcgctggggacgtcgagtccaatccagggcccatggatagcaaaggctctagccagaaaggcagccgactcctgctcctgctggtcgtcagtaacctgctgctgccccagggcgtcctcgccaagacaagaggcaagctgtgccccgactgcctgaattgcaccgacctggatgtggccctgggcagacctatgtgcgtgggcacaacacctagcgccaaggccagcatcctgcacgaagtgcggcctgtgaccagcggctgcttccctatcatgcacgaccggaccaagatcaggcagctggccaatctgctgagaggctacgagaacatccggctgagcacccagaatgtgatcgatgccgagaaggcccctggcggcccttacagactgggcacaagcggcagctgtcccaacgccaccagcaagagcggctttttcgccacaatggcctgggccgtgcccaaggacaacaacaagaatgccaccaaccctctgaccgtggaagtgccctacatctgcgccgagggcgaggatcagatcacagtgtggggcttccacagcgacgacaagacccagatgaagaacctgtacggcgacagcaatccccagaagttcacctccagcgccaatggcgtgaccacccactacgtgtcccagatcggcggcttccccgatcagacagaggatggcggactgccccagtccggcagaatcgtggtggactacatgatgcagaagcccggcaagaccggcaccatcgtgtaccagagaggcgtgctgctccctcagaaagtgtggtgcgcctctggcagaagctccggagaatctcaagtccgccagcagttttctaaggacatcgaaaagctgctcaatgaacaggtcaacaaagagatgcagtcctctaacctgtatatgagtatgagttcctggtgctatacccactctctcgacggtgcagggctgttcctcttcgaccacgctgcagaggaatatgaacatgctaagaaactgattatctttctcaacgaaaacaacgtgccagtccagctcaccagtatctctgcccctgaacataagtttgaggggctcactcagatctttcagaaagcttacgagcacgagcagcatatctctgagtctattaacaacatcgtcgaccatgctatcaaatctaaagaccacgctacttttaactttctccaatggtacgtcgcagagcagcatgaggaagaggtcctcttcaaggacattctcgacaaaattgaactcatcggaaacgaaaaccatgggctctacctggctgatcagtacgtcaagggaatcgcaaaaagccggaagtcttgatga

SEQ ID NO: 178 - protein, 1335 aa, synthetic construct
  REGION 1..1335: note = Synthetic
MDSKGSSQKGSRLLLLLVVSNLLLPQGVLAVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSRKRRSGSGAPVKQTLNFDLLKLAGDVESNPGPMDSKGSSQKGSRLLLLLVVSNLLLPQGVLAHQILDGENCTLIDALLGDPQCDGFQNKKWDLFVERSKAYSNCFPYDVPDYASLRSLVASSGTLEFNNESFNWNGVTQNGTSSACIRRSNNSFFSRLNWLTHLNFKYPALNVTMPNNEQFDKLYIWGVHHPVTDKDQIFLYAQPSGRITVSTKRSQQAVIPNIGFRPRIRNIPSRISIYWTIVKPGDILLINSTGNLIAPRGYFKIRSGASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSRKRRSGSGAPVKQTLNFDLLKLAGDVESNPGPMDSKGSSQKGSRLLLLLVVSNLLLPQGVLAKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 179 - DNA, 4011 bp, synthetic construct
  misc_feature 1..4011: note = Synthetic
  source 1..4011: mol_type = other DNA
tcatcaagacttccggctttttgcgattcccttgacgtactgatcagccaggtagagcccatggttttcgtttccgatgagttcaattttgtcgagaatgtccttgaagaggacctcttcctcatgctgctctgcgacgtaccattggagaaagttaaaagtagcgtggtctttagatttgatagcatggtcgacgatgttgttaatagactcagagatatgctgctcgtgctcgtaagctttctgaaagatctgagtgagcccctcaaacttatgttcaggggcagagatactggtgagctggactggcacgttgttttcgttgagaaagataatcagtttcttagcatgttcatattcctctgcagcgtggtcgaagaggaacagccctgcaccgtcgagagagtgggtatagcaccaggaactcatactcatatacaggttagaggactgcatctctttgttgacctgttcattgagcagcttttcgatgtccttagaaaactgctggcggacttgagattctccggagcttctgccagaggcgcaccacactttctgagggagcagcacgcctctctggtacacgatggtgccggtcttgccgggcttctgcatcatgtagtccaccacgattctgccggactggggcagtccgccatcctctgtctgatcggggaagccgccgatctgggacacgtagtgggtggtcacgccattggcgctggaggtgaacttctggggattgctgtcgccgtacaggttcttcatctgggtcttgtcgtcgctgtggaagccccacactgtgatctgatcctcgccctcggcgcagatgtagggcacttccacggtcagagggttggtggcattcttgttgttgtccttgggcacggcccaggccattgtggcgaaaaagccgctcttgctggtggcgttgggacagctgccgcttgtgcccagtctgtaagggccgccaggggccttctcggcatcgatcacattctgggtgctcagccggatgttctcgtagcctctcagcagattggccagctgcctgatcttggtccggtcgtgcatgatagggaagcagccgctggtcacaggccgcacttcgtgcaggatgctggccttggcgctaggtgttgtgcccacgcacataggtctgcccagggccacatccaggtcggtgcaattcaggcagtcggggcacagcttgcctcttgtcttggcgaggacgccctggggcagcagcaggttactgacgaccagcaggagcaggagtcggctgcctttctggctagagcctttgctatccatgggccctggattggactcgacgtccccagcgagcttgagcagatcaaaattcagtgtctgtttgactggggcgccagagccgcttctccgctttctggacttccgggacttagcgatccctttcacatactgatcggcgagatagagcccgtgattctcattcccaatgagttcgatcttatcgaggatatctttgaacaggacttcctcttcatgctgctcagcgacataccattggaggaaattgaatgtagcatggtccttggacttaatggcatgatcgacaatgttgttgatggactcggaaatgtgctgttcatgctcataggctttctgaaaaatctgtgtgagtccttcaaatttgtgctcgggagcggagatggaggtgagctggacagggacattgttctcattgagaaaaatgatgagttttttggcgtgttcatactcttcagcggcatggtcgaacagaaacagtccggccccatccagggagtgtgtgtagcaccaagaggacatagacatgtagaggttggaggactgcatttccttgttgacctgttcattgaggagtttctcaatatccttggaaaactgctggcgcacttgggactctccggaggcgccgcttctgatcttgaagtagccccgaggggcgatcaggttgccggtgctgttgatcagcaggatgtcgccaggcttcacgattgtccagtagatgctgatccggctggggatgtttctgatcctgggccggaagccgatgttggggatcacggcctgctggcttctcttggtagacacggtgatccggccgctgggctgggcgtacagaaagatctggtccttgtcggtcacggggtggtgcactccccagatgtacagcttgtcgaactgctcattgttgggcatggtcacgttcagggcggggtacttgaaattcaggtgggtcagccagttcagccgggagaagaagctgttgttgcttcttctgatgcaggcggagctggtgccgttctgggtcacgccgttccagttgaagctctcgttgttgaattccagggtgccgctgctggccaccaggcttctcaggctggcgtagtcgggcacgtcgtaggggaagcagttgctgtaggccttgctccgctcgacgaacaggtcccacttcttgttctggaagccgtcgcactgagggtcgcccagcagggcgtcgatcagggtgcagttctcgccatccaggatctggtgggccaggacaccctgtggcagcagcagattagacaccacgagcaggaggagcagccgagatcctttctgggaggagcccttggagtccatgggcccagggttgctttccacgtcgccggccagcttcagcaggtcgaagttcagggtctgcttcacaggagcgccgctgccagatctccgcttccggctctttctgctcttggcaatgcccttcacgtactggtcggccaggtacaggccgtggttctcgttgccgatcagctcgatcttgtccaggatgtccttaaacagcacttcttcctcgtgctgttcggccacgtaccattgcagaaagttgaaggtggcgtgatccttgctcttgatggcgtggtccacgatattgttgatgctctcgctgatgtgctgctcgtgttcgtaggccttctggaagatctgggtcaggccctcgaacttgtgctctggggcgctgatgctggtcagctgcacgggcacgttgttctcgttcaggaagatgatcagcttcttggcgtgctcgtattcctcggcggcgtgatcaaacaggaacaggccagcgccgtccaggctgtgggtgtaacaccagctggacatgctcatgtacaggttgctgctctgcatctctttgttgacctgctcgttcagcagcttctcgatgtccttgctgaactgctgtcggacttggctctcgccgctggcgtttctttccatggcgaaggcgtatctgggcaccaccagattgccggtggcctcgaaggtgatcttgtcgccgggttccaccagggtccagtagtagttcatccggccttcctggtcccgcactttgggccggatggcgatctcgggcttgaacttcttgctgtaccgggagctgcccacgaacacgtaggcgtcggcgttctggtacaggctctgctggtcggcgcttgtgctagggtggtggatgccccagaggaccagcacctctttgcccttgtcgttgatgtagctcttgctcagcttggggtagctgttgcccttcttgaccagccagatcaggttcttgtagaagctcttggcgccagcgtgaggacaggcggcggtcacgcctttgttgctgtcgtggttgggccagctggaggtcttggggaagatctcgaatctctcgaagctggacacgctgctcagctgctcgcgcagttcctcgtagtcgatgaagtcgccggggaaacaggtgccgttgtcgctgctaggggtttccacgatgtaggaccagctgctggctgtgctcaggctctcgcactcggggttgcccagaatccagccggcgatattgcacttgcccaggtgcagaggagccacggctagcacgccctgaggcagcaggaggttggacaccaccaggagcagcagcagtctggagcccttctggctgctgcccttgctgtccat

SEQ ID NO: 180 - DNA, 1518 bp, synthetic construct
  misc_feature 1..1518: note = Synthetic
  source 1..1518: mol_type = other DNA
atgcccatgggcagcctgcagcccctggccaccctgtacctgctgggcatgctggtggctagcgtgctggccatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaattgcccctctgcagctgggcaattgttctgtggccggatggattctgggcaaccccgagtgtgagctgctgatttctaaggagagctggagctacatcgtggagacccccaatcctgagaatggcacctgctaccctggctacttcgccgattacgaggagctgcgcgagcagctgtctagcgtgtccagcttcgagagattcgagatcttccccaaggagtccagctggcctaatcacacagtgacaggcgtgtctgccagctgtagccacaacggcaaaagcagcttctaccggaacctgctgtggctgacaggcaagaatggcctgtaccccaacctgagcaagagctacgtgaacaacaaggaaaaggaagtgctggtgctgtggggagtgcaccaccctcccaacatcggaaatcagcgggccctgtaccacacagagaacgcctatgtgagcgtggtgtccagccactacagcagaagattcacccccgagatcgccaagagacccaaagtgagagaccaggagggccggatcaattactactggaccctgctggagcctggcgataccatcatcttcgaggccaacggcaatctgatcgccccttggtatgcctttgccctgagcagaggcgcctgatga

SEQ ID NO: 181 - protein, 504 aa, synthetic construct
  REGION 1..504: note = Synthetic
MPMGSLQPLATLYLLGMLVASVLAMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGA

SEQ ID NO: 182 - DNA, 1518 bp, synthetic construct
  misc_feature 1..1518: note = Synthetic
  source 1..1518: mol_type = other DNA
tcatcaggcgcctctgctcagggcaaaggcataccaaggggcgatcagattgccgttggcctcgaagatgatggtatcgccaggctccagcagggtccagtagtaattgatccggccctcctggtctctcactttgggtctcttggcgatctcgggggtgaatcttctgctgtagtggctggacaccacgctcacataggcgttctctgtgtggtacagggcccgctgatttccgatgttgggagggtggtgcactccccacagcaccagcacttccttttccttgttgttcacgtagctcttgctcaggttggggtacaggccattcttgcctgtcagccacagcaggttccggtagaagctgcttttgccgttgtggctacagctggcagacacgcctgtcactgtgtgattaggccagctggactccttggggaagatctcgaatctctcgaagctggacacgctagacagctgctcgcgcagctcctcgtaatcggcgaagtagccagggtagcaggtgccattctcaggattgggggtctccacgatgtagctccagctctccttagaaatcagcagctcacactcggggttgcccagaatccatccggccacagaacaattgcccagctgcagaggggcaattccggacttcagcaggatcagggcctcggggttcaccacctggaaggtgaaggtctcggtgatgaacagcctcacggcgtccttctccctgtcctcgtagccgatgctcaggtcctggcccaggatcagcttgaagtcgccgcccctctcgctcaccaccagggcgtcctcgatcctgggggtggtgatgatcttgccgcccctcaggcactcctccaccctcttctccagggggtagtggccggcctcctccttcaggaagttgatccacctgtcggtgttgatcaccagggtgtaggggccctcgatgccgtccttgctgaagatgctcagggccctcacgatggcctccagcaggtccttgggggtgctgccgcactcgatcttcctctcctcgaagctcagcaggcccttcacgccgctcttctcgcagcccctgaagatcacctcgtcctcgaactcggccaccttcctcacggtctcctccaggctgctcaggtccacgttgggcttgcccctctccaggttgtccagctcccacaggtccagggtgaaggtggccctcagctcgatcaggggcaggctcttcctcaggccccacttcaccacctcgttctcgtcgctcagcacctccacctcgcccagggggtgggcggcgtactcccagccgtaggggccctccacgtccacgaacttcctgccgtacagctgggtcttgaagatctccctggccctgttgtcgatctcctgccactgcttctcggtcagaggggcgaagctcctcttcaggaactccatggccagcacgctagccaccagcatgcccagcaggtacagggtggccaggggctgcaggctgcccatgggcat

SEQ ID NO: 183 - DNA, 1521 bp, synthetic construct
  misc_feature 1..1521: note = Synthetic
  source 1..1521: mol_type = other DNA
atgcccatgggcagcctgcagcccctggccaccctgtacctgctgggcatgctggtggctagcgtgctggccatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggagtggcccccctgcacctgggcaagtgcaacatcgccggctggattctgggcaaccccgagtgcgagagcctgagcaccgccagcagctggagctacatcgtggagacccccagcagcgacaacggcacctgctaccccggcgacttcatcgactacgaggagctgcgggagcagctgagcagcgtgagcagcttcgagcggttcgagatcttccccaagaccagcagctggcccaaccacgacagcaacaagggcgtgaccgccgcctgcccccacgccggcgccaagagcttctacaagaacctgatctggctggtgaagaagggcaacagctaccccaagctgagcaagagctacatcaacgacaagggcaaggaggtgctggtgctgtggggcatccaccaccccagcaccagcgccgaccagcagagcctgtaccagaacgccgacacctacgtgttcgtgggcagcagccggtacagcaagaagttcaagcccgagatcgccatccggcccaaggtgcgggaccaggagggccggatgaactactactggaccctggtggagcccggcgacaagatcaccttcgaggccaccggcaacctggtggtgccccggtacgccttcgccatggagcggaacgcctgatga

SEQ ID NO: 184 - protein, 505 aa, synthetic construct
  REGION 1..505: note = Synthetic
MPMGSLQPLATLYLLGMLVASVLAMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNA

SEQ ID NO: 185 - DNA, 1521 bp, synthetic construct
  misc_feature 1..1521: note = Synthetic
  source 1..1521: mol_type = other DNA
tcatcaggcgttccgctccatggcgaaggcgtaccggggcaccaccaggttgccggtggcctcgaaggtgatcttgtcgccgggctccaccagggtccagtagtagttcatccggccctcctggtcccgcaccttgggccggatggcgatctcgggcttgaacttcttgctgtaccggctgctgcccacgaacacgtaggtgtcggcgttctggtacaggctctgctggtcggcgctggtgctggggtggtggatgccccacagcaccagcacctccttgcccttgtcgttgatgtagctcttgctcagcttggggtagctgttgcccttcttcaccagccagatcaggttcttgtagaagctcttggcgccggcgtgggggcaggcggcggtcacgcccttgttgctgtcgtggttgggccagctgctggtcttggggaagatctcgaaccgctcgaagctgctcacgctgctcagctgctcccgcagctcctcgtagtcgatgaagtcgccggggtagcaggtgccgttgtcgctgctgggggtctccacgatgtagctccagctgctggcggtgctcaggctctcgcactcggggttgcccagaatccagccggcgatgttgcacttgcccaggtgcaggggggccactccggacttcagcaggatcagggcctcggggttcaccacctggaaggtgaaggtctcggtgatgaacagcctcacggcgtccttctccctgtcctcgtagccgatgctcaggtcctggcccaggatcagcttgaagtcgccgcccctctcgctcaccaccagggcgtcctcgatcctgggggtggtgatgatcttgccgcccctcaggcactcctccaccctcttctccagggggtagtggccggcctcctccttcaggaagttgatccacctgtcggtgttgatcaccagggtgtaggggccctcgatgccgtccttgctgaagatgctcagggccctcacgatggcctccagcaggtccttgggggtgctgccgcactcgatcttcctctcctcgaagctcagcaggcccttcacgccgctcttctcgcagcccctgaagatcacctcgtcctcgaactcggccaccttcctcacggtctcctccaggctgctcaggtccacgttgggcttgcccctctccaggttgtccagctcccacaggtccagggtgaaggtggccctcagctcgatcaggggcaggctcttcctcaggccccacttcaccacctcgttctcgtcgctcagcacctccacctcgcccagggggtgggcggcgtactcccagccgtaggggccctccacgtccacgaacttcctgccgtacagctgggtcttgaagatctccctggccctgttgtcgatctcctgccactgcttctcggtcagaggggcgaagctcctcttcaggaactccatggccagcacgctagccaccagcatgcccagcaggtacagggtggccaggggctgcaggctgcccatgggcat

SEQ ID NO: 186 - DNA, 4359 bp, synthetic construct
  misc_feature 1..4359: note = Synthetic
  source 1..4359: mol_type = other DNA
atggagttcatcccgacgcaaactttctataacagaaggtaccaaccccgaccctgggccccacgccctacaattcaagtaattagacctagaccacgtccacagaggcaggctgggcaactcgcccagctgatctccgcagtcaacaaattgaccatgcgcgcggtacctcaacagaagcctcgcagaaatcggaaaaacaagaagcaaaggcagaagaagcaggcgccgcaaaacgacccaaagcaaaagaagcaaccaccacaaaagaagccggctcaaaagaagaagaaaccaggccgtagggagagaatgtgcatgaaaattgaaaatgattgcatcttcgaagtcaagcatgaaggcaaagtgatgggctacgcatgcctggtgggggataaagtaatgaaaccagcacatgtgaagggaactatcgacaatgccgatctggctaaactggcctttaagcggtcgtctaaatacgatcttgaatgtgcacagataccggtgcacatgaagtctgatgcctcgaagtttacccacgagaaacccgaggggtactataactggcatcacggagcagtgcagtattcaggaggccggttcactatcccgacgggtgcaggcaagccgggagacagcggcagaccgatcttcgacaacaaaggacgggtggtggccatcgtcctaggaggggccaacgaaggtgcccgcacggccctctccgtggtgacgtggaacaaagacatcgtcacaaaaattacccctgagggagccgaagagtggagcctcgccctcccggtcttgtgcctgttggcaaacactacattcccctgctctcagccgccttgcacaccctgctgctacgaaaaggaaccggaaagcaccttgcgcatgcttgaggacaacgtgatgagacccggatactaccagctactaaaagcatcgctgacttgccgccaaagacgcctgcagctggggaattgttctgtggccggatggattctgggcaaccccgagtgtgagctgctgatttctaaggagagctggagctacatcgtggagacccccaatcctgagaatggcacctgcttccctggctacttcgccgattacgaggagctgcgcgagcagctgtctagcgtgtccagcttcgagagattcgagatcttccccaaggagtccagctggcctaatcacacagtgacaggcgtgtctgccagctgtagccacaacggcaaaagcagcttctaccggaacctgctgtggctgacaggcaagaatggcctgtaccccaacctgagcaagagctacgtgaacaacaaggaaaaggaagtgctggtgctgtggggagtgcaccaccctcccaacatcggaaatcagcgggccctgtaccacacagagaacgcctatgtgagcgtggtgtccagccactacagcagaagattcacccccgagatcgccaagagacccaaagtgagagaccaggagggccggatcaattactactggaccctgctggagcctggcgataccatcatcttcgaggccaacggcaatctgatcgccccttggtatgcctttgccctgagcagaggcgccaattttaatgtctataaagccacaagaccatatctagctcattgtcctgactgcggagaagggcattcgtgccacagccctatcgcattggagcgcatcagaaatgaagcaacggacggaacgctgaaaatccaggtctctttgcagatcgggataaagacagatgacagccacgattggaccaagctgcgctatatggatagccatacgccagcggacgcggagcgagccggattgcttgtaaggacttcagcaccgtgcacgatcaccgggaccatgggacactttattctcgcccgatgcccgaaaggagagacgctgacagtgggatttacggacagcagaaagatcagccacacatgcacacacccgttccatcatgaaccacctgtgataggtagggagaggttccactctcgaccacaacatggtaaagagttaccttgcagcacgtacgtgcagagcaccgctgccactgctgaggagatagaggtgcatatgcccccagatactcctgaccgcacgctgatgacgcagcagtctggcaacgtgaagatcacagttaatgggcagacggtgcggtacaagtgcaactgcggtggctcaaacgagggactgacaaccacagacaaagtgatcaataactgcaaaattgatcagtgccatgctgcagtcactaatcacaagaattggcaatacaactcccctttagtcccgcgcaacgctgaactcggggaccgtaaaggaaagatccacatcccattcccattggcaaacgtgacttgcagagtgccaaaagcaagaaaccctacagtaacttacggaaaaaaccaagtcaccatgctgctgtatcctgaccatccgacactcttgtcttaccgtaacatgggacaggaaccaaattaccacgaggagtgggtgacacacaagaaggaggttaccttgaccgtgcctactgagggtctggaggtcacttggggcaacaacgaaccatacaagtactggccgcagatgtctacgaacggtactgctcatggtcacccacatgagataatcttgtactattatgagctgtaccccactatgactgtagtcattgtgtcggtggcctcgttcgtgcttctgtcgatggtgggcacagcagtgggaatgtgtgtgtgcgcacggcgcagatgcattacaccatatgaattaacaccaggagccactgttcccttcctgctcagcctgctatgctgcgtcagaacgaccaaggcggccacatattacgaggctgcggcatatctatggaacgaacagcagcccctgttctggttgcaggctcttatcccgctggccgccttgatcgtcctgtgcaactgtctgaaactcttgccatgctgctgtaagaccctggcttttttagccgtaatgagcatcggtgcccacactgtgagcgcgtacgaacacgtaacagtgatcccgaacacggtgggagtaccgtataagactcttgtcaacagaccgggttacagccccatggtgttggagatggagctacaatcagtcaccttggaaccaacactgtcacttgactacatcacgtgcgagtacaaaactgtcatcccctccccgtacgtgaagtgctgtggtacagcagagtgcaaggacaagagcctaccagactacagctgcaaggtctttactggagtctacccatttatgtggggcggcgcctactgcttttgcgacgccgaaaatacgcaattgagcgaggcacatgtagagaaatctgaatcttgcaaaacagagtttgcatcggcctacagagcccacaccgcatcggcgtcggcgaagctccgcgtcctttaccaaggaaacaacattaccgtagctgcctacgctaacggtgaccatgccgtcacagtaaaggacgccaagtttgtcgtgggcccaatgtcctccgcctggacaccttttgacaacaaaatcgtggtgtacaaaggcgacgtctacaacatggactacccaccttttggcgcaggaagaccaggacaatttggtgacattcaaagtcgtacaccggaaagtaaagacgtttatgccaacactcagttggtactacagaggccagcagcaggcacggtacatgtaccatactctcaggcaccatctggcttcaagtattggctgaaggaacgaggagcatcgctacagcacacggcaccgttcggttgccagattgcgacaaacccggtaagagctgtaaattgcgctgtggggaacataccaatttccatcgacataccggatgcggcctttactagggttgtcgatgcaccctctgtaacggacatgtcatgcgaagtaccagcctgcactcactcctccgactttgggggcgtcgccatcatcaaatacacagctagcaagaaaggtaaatgtgcagtacattcgatgaccaacgccgttaccattcgagaagccgacgtagaagtagaggggaactcccagctgcaaatatccttctcaacagccctggcaagcgccgagtttcgcgtgcaagtgtgctccacacaagtacactgcgcagccgcatgccaccctccaaaggaccacatagtcaattacccagcatcacacaccacccttggggtccaggatatatccacaacggcaatgtcttgggtgcagaagattacgggaggagtaggattaattgttgctgttgctgccttaattttaattgtggtgctatgcgtgtcgtttagcaggcactaa

SEQ ID NO: 187 - protein, 1452 aa, synthetic construct
  REGION 1..1452: note = Synthetic
MEFIPTQTFYNRRYQPRPWAPRPTIQVIRPRPRPQRQAGQLAQLISAVNKLTMRAVPQQKPRRNRKNKKQRQKKQAPQNDPKQKKQPPQKKPAQKKKKPGRRERMCMKIENDCIFEVKHEGKVMGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLALPVLCLLANTTFPCSQPPCTPCCYEKEPESTLRMLEDNVMRPGYYQLLKASLTCRQRRLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGANFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKAATYYEAAAYLWNEQQPLFWLQALIPLAALIVLCNCLKLLPCCCKTLAFLAVMSIGAHTVSAYEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 188 - DNA, 4359 bp, synthetic construct
  misc_feature 1..4359: note = Synthetic
  source 1..4359: mol_type = other DNA
ttagtgcctgctaaacgacacgcatagcaccacaattaaaattaaggcagcaacagcaacaattaatcctactcctcccgtaatcttctgcacccaagacattgccgttgtggatatatcctggaccccaagggtggtgtgtgatgctgggtaattgactatgtggtcctttggagggtggcatgcggctgcgcagtgtacttgtgtggagcacacttgcacgcgaaactcggcgcttgccagggctgttgagaaggatatttgcagctgggagttcccctctacttctacgtcggcttctcgaatggtaacggcgttggtcatcgaatgtactgcacatttacctttcttgctagctgtgtatttgatgatggcgacgcccccaaagtcggaggagtgagtgcaggctggtacttcgcatgacatgtccgttacagagggtgcatcgacaaccctagtaaaggccgcatccggtatgtcgatggaaattggtatgttccccacagcgcaatttacagctcttaccgggtttgtcgcaatctggcaaccgaacggtgccgtgtgctgtagcgatgctcctcgttccttcagccaatacttgaagccagatggtgcctgagagtatggtacatgtaccgtgcctgctgctggcctctgtagtaccaactgagtgttggcataaacgtctttactttccggtgtacgactttgaatgtcaccaaattgtcctggtcttcctgcgccaaaaggtgggtagtccatgttgtagacgtcgcctttgtacaccacgattttgttgtcaaaaggtgtccaggcggaggacattgggcccacgacaaacttggcgtcctttactgtgacggcatggtcaccgttagcgtaggcagctacggtaatgttgtttccttggtaaaggacgcggagcttcgccgacgccgatgcggtgtgggctctgtaggccgatgcaaactctgttttgcaagattcagatttctctacatgtgcctcgctcaattgcgtattttcggcgtcgcaaaagcagtaggcgccgccccacataaatgggtagactccagtaaagaccttgcagctgtagtctggtaggctcttgtccttgcactctgctgtaccacagcacttcacgtacggggaggggatgacagttttgtactcgcacgtgatgtagtcaagtgacagtgttggttccaaggtgactgattgtagctccatctccaacaccatggggctgtaacccggtctgttgacaagagtcttatacggtactcccaccgtgttcgggatcactgttacgtgttcgtacgcgctcacagtgtgggcaccgatgctcattacggctaaaaaagccagggtcttacagcagcatggcaagagtttcagacagttgcacaggacgatcaaggcggccagcgggataagagcctgcaaccagaacaggggctgctgttcgttccatagatatgccgcagcctcgtaatatgtggccgccttggtcgttctgacgcagcatagcaggctgagcaggaagggaacagtggctcctggtgttaattcatatggtgtaatgcatctgcgccgtgcgcacacacacattcccactgctgtgcccaccatcgacagaagcacgaacgaggccaccgacacaatgactacagtcatagtggggtacagctcataatagtacaagattatctcatgtgggtgaccatgagcagtaccgttcgtagacatctgcggccagtacttgtatggttcgttgttgccccaagtgacctccagaccctcagtaggcacggtcaaggtaacctccttcttgtgtgtcacccactcctcgtggtaatttggttcctgtcccatgttacggtaagacaagagtgtcggatggtcaggatacagcagcatggtgacttggttttttccgtaagttactgtagggtttcttgcttttggcactctgcaagtcacgtttgccaatgggaatgggatgtggatctttcctttacggtccccgagttcagcgttgcgcgggactaaaggggagttgtattgccaattcttgtgattagtgactgcagcatggcactgatcaattttgcagttattgatcactttgtctgtggttgtcagtccctcgtttgagccaccgcagttgcacttgtaccgcaccgtctgcccattaactgtgatcttcacgttgccagactgctgcgtcatcagcgtgcggtcaggagtatctgggggcatatgcacctctatctcctcagcagtggcagcggtgctctgcacgtacgtgctgcaaggtaactctttaccatgttgtggtcgagagtggaacctctccctacctatcacaggtggttcatgatggaacgggtgtgtgcatgtgtggctgatctttctgctgtccgtaaatcccactgtcagcgtctctcctttcgggcatcgggcgagaataaagtgtcccatggtcccggtgatcgtgcacggtgctgaagtccttacaagcaatccggctcgctccgcgtccgctggcgtatggctatccatatagcgcagcttggtccaatcgtggctgtcatctgtctttatcccgatctgcaaagagacctggattttcagcgttccgtccgttgcttcatttctgatgcgctccaatgcgatagggctgtggcacgaatgcccttctccgcagtcaggacaatgagctagatatggtcttgtggctttatagacattaaaattggcgcctctgctcagggcaaaggcataccaaggggcgatcagattgccgttggcctcgaagatgatggtatcgccaggctccagcagggtccagtagtaattgatccggccctcctggtctctcactttgggtctcttggcgatctcgggggtgaatcttctgctgtagtggctggacaccacgctcacataggcgttctctgtgtggtacagggcccgctgatttccgatgttgggagggtggtgcactccccacagcaccagcacttccttttccttgttgttcacgtagctcttgctcaggttggggtacaggccattcttgcctgtcagccacagcaggttccggtagaagctgcttttgccgttgtggctacagctggcagacacgcctgtcactgtgtgattaggccagctggactccttggggaagatctcgaatctctcgaagctggacacgctagacagctgctcgcgcagctcctcgtaatcggcgaagtagccagggaagcaggtgccattctcaggattgggggtctccacgatgtagctccagctctccttagaaatcagcagctcacactcggggttgcccagaatccatccggccacagaacaattccccagctgcaggcgtctttggcggcaagtcagcgatgcttttagtagctggtagtatccgggtctcatcacgttgtcctcaagcatgcgcaaggtgctttccggttccttttcgtagcagcagggtgtgcaaggcggctgagagcaggggaatgtagtgtttgccaacaggcacaagaccgggagggcgaggctccactcttcggctccctcaggggtaatttttgtgacgatgtctttgttccacgtcaccacggagagggccgtgcgggcaccttcgttggcccctcctaggacgatggccaccacccgtcctttgttgtcgaagatcggtctgccgctgtctcccggcttgcctgcacccgtcgggatagtgaaccggcctcctgaatactgcactgctccgtgatgccagttatagtacccctcgggtttctcgtgggtaaacttcgaggcatcagacttcatgtgcaccggtatctgtgcacattcaagatcgtatttagacgaccgcttaaaggccagtttagccagatcggcattgtcgatagttcccttcacatgtgctggtttcattactttatcccccaccaggcatgcgtagcccatcactttgccttcatgcttgacttcgaagatgcaatcattttcaattttcatgcacattctctccctacggcctggtttcttcttcttttgagccggcttcttttgtggtggttgcttcttttgctttgggtcgttttgcggcgcctgcttcttctgcctttgcttcttgtttttccgatttctgcgaggcttctgttgaggtaccgcgcgcatggtcaatttgttgactgcggagatcagctgggcgagttgcccagcctgcctctgtggacgtggtctaggtctaattacttgaattgtagggcgtggggcccagggtcggggttggtaccttctgttatagaaagtttgcgtcgggatgaactccat

SEQ ID NO: 189 - DNA, 4362 bp, synthetic construct
  misc_feature 1..4362: note = Synthetic
  source 1..4362: mol_type = other DNA
atggagttcatcccgacgcaaactttctataacagaaggtaccaaccccgaccctgggccccacgccctacaattcaagtaattagacctagaccacgtccacagaggcaggctgggcaactcgcccagctgatctccgcagtcaacaaattgaccatgcgcgcggtacctcaacagaagcctcgcagaaatcggaaaaacaagaagcaaaggcagaagaagcaggcgccgcaaaacgacccaaagcaaaagaagcaaccaccacaaaagaagccggctcaaaagaagaagaaaccaggccgtagggagagaatgtgcatgaaaattgaaaatgattgcatcttcgaagtcaagcatgaaggcaaagtgatgggctacgcatgcctggtgggggataaagtaatgaaaccagcacatgtgaagggaactatcgacaatgccgatctggctaaactggcctttaagcggtcgtctaaatacgatcttgaatgtgcacagataccggtgcacatgaagtctgatgcctcgaagtttacccacgagaaacccgaggggtactataactggcatcacggagcagtgcagtattcaggaggccggttcactatcccgacgggtgcaggcaagccgggagacagcggcagaccgatcttcgacaacaaaggacgggtggtggccatcgtcctaggaggggccaacgaaggtgcccgcacggccctctccgtggtgacgtggaacaaagacatcgtcacaaaaattacccctgagggagccgaagagtggagcctcgccctcccggtcttgtgcctgttggcaaacactacattcccctgctctcagccgccttgcacaccctgctgctacgaaaaggaaccggaaagcaccttgcgcatgcttgaggacaacgtgatgagacccggatactaccagctactaaaagcatcgctgacttgccgccaaagacgcctgcacctgggcaagtgcaacatcgccggctggattctgggcaaccccgagtgcgagagcctgagcaccgccagcagctggagctacatcgtggagacccccagcagcgacaacggcacctgcttccccggcgacttcatcgactacgaggagctgcgggagcagctgagcagcgtgagcagcttcgagcggttcgagatcttccccaagaccagcagctggcccaaccacgacagcaacaagggcgtgaccgccgcctgcccccacgccggcgccaagagcttctacaagaacctgatctggctggtgaagaagggcaacagctaccccaagctgagcaagagctacatcaacgacaagggcaaggaggtgctggtgctgtggggcatccaccaccccagcaccagcgccgaccagcagagcctgtaccagaacgccgacacctacgtgttcgtgggcagcagccggtacagcaagaagttcaagcccgagatcgccatccggcccaaggtgcgggaccaggagggccggatgaactactactggaccctggtggagcccggcgacaagatcaccttcgaggccaccggcaacctggtggtgccccggtacgccttcgccatggagcggaacgccaattttaatgtctataaagccacaagaccatatctagctcattgtcctgactgcggagaagggcattcgtgccacagccctatcgcattggagcgcatcagaaatgaagcaacggacggaacgctgaaaatccaggtctctttgcagatcgggataaagacagatgacagccacgattggaccaagctgcgctatatggatagccatacgccagcggacgcggagcgagccggattgcttgtaaggacttcagcaccgtgcacgatcaccgggaccatgggacactttattctcgcccgatgcccgaaaggagagacgctgacagtgggatttacggacagcagaaagatcagccacacatgcacacacccgttccatcatgaaccacctgtgataggtagggagaggttccactctcgaccacaacatggtaaagagttaccttgcagcacgtacgtgcagagcaccgctgccactgctgaggagatagaggtgcatatgcccccagatactcctgaccgcacgctgatgacgcagcagtctggcaacgtgaagatcacagttaatgggcagacggtgcggtacaagtgcaactgcggtggctcaaacgagggactgacaaccacagacaaagtgatcaataactgcaaaattgatcagtgccatgctgcagtcactaatcacaagaattggcaatacaactcccctttagtcccgcgcaacgctgaactcggggaccgtaaaggaaagatccacatcccattcccattggcaaacgtgacttgcagagtgccaaaagcaagaaaccctacagtaacttacggaaaaaaccaagtcaccatgctgctgtatcctgaccatccgacactcttgtcttaccgtaacatgggacaggaaccaaattaccacgaggagtgggtgacacacaagaaggaggttaccttgaccgtgcctactgagggtctggaggtcacttggggcaacaacgaaccatacaagtactggccgcagatgtctacgaacggtactgctcatggtcacccacatgagataatcttgtactattatgagctgtaccccactatgactgtagtcattgtgtcggtggcctcgttcgtgcttctgtcgatggtgggcacagcagtgggaatgtgtgtgtgcgcacggcgcagatgcattacaccatatgaattaacaccaggagccactgttcccttcctgctcagcctgctatgctgcgtcagaacgaccaaggcggccacatattacgaggctgcggcatatctatggaacgaacagcagcccctgttctggttgcaggctcttatcccgctggccgccttgatcgtcctgtgcaactgtctgaaactcttgccatgctgctgtaagaccctggcttttttagccgtaatgagcatcggtgcccacactgtgagcgcgtacgaacacgtaacagtgatcccgaacacggtgggagtaccgtataagactcttgtcaacagaccgggttacagccccatggtgttggagatggagctacaatcagtcaccttggaaccaacactgtcacttgactacatcacgtgcgagtacaaaactgtcatcccctccccgtacgtgaagtgctgtggtacagcagagtgcaaggacaagagcctaccagactacagctgcaaggtctttactggagtctacccatttatgtggggcggcgcctactgcttttgcgacgccgaaaatacgcaattgagcgaggcacatgtagagaaatctgaatcttgcaaaacagagtttgcatcggcctacagagcccacaccgcatcggcgtcggcgaagctccgcgtcctttaccaaggaaacaacattaccgtagctgcctacgctaacggtgaccatgccgtcacagtaaaggacgccaagtttgtcgtgggcccaatgtcctccgcctggacaccttttgacaacaaaatcgtggtgtacaaaggcgacgtctacaacatggactacccaccttttggcgcaggaagaccaggacaatttggtgacattcaaagtcgtacaccggaaagtaaagacgtttatgccaacactcagttggtactacagaggccagcagcaggcacggtacatgtaccatactctcaggcaccatctggcttcaagtattggctgaaggaacgaggagcatcgctacagcacacggcaccgttcggttgccagattgcgacaaacccggtaagagctgtaaattgcgctgtggggaacataccaatttccatcgacataccggatgcggcctttactagggttgtcgatgcaccctctgtaacggacatgtcatgcgaagtaccagcctgcactcactcctccgactttgggggcgtcgccatcatcaaatacacagctagcaagaaaggtaaatgtgcagtacattcgatgaccaacgccgttaccattcgagaagccgacgtagaagtagaggggaactcccagctgcaaatatccttctcaacagccctggcaagcgccgagtttcgcgtgcaagtgtgctccacacaagtacactgcgcagccgcatgccaccctccaaaggaccacatagtcaattacccagcatcacacaccacccttggggtccaggatatatccacaacggcaatgtcttgggtgcagaagattacgggaggagtaggattaattgttgctgttgctgccttaattttaattgtggtgctatgcgtgtcgtttagcaggcactaa

SEQ ID NO: 190 - protein, 1453 aa, synthetic construct
  REGION 1..1453: note = Synthetic
MEFIPTQTFYNRRYQPRPWAPRPTIQVIRPRPRPQRQAGQLAQLISAVNKLTMRAVPQQKPRRNRKNKKQRQKKQAPQNDPKQKKQPPQKKPAQKKKKPGRRERMCMKIENDCIFEVKHEGKVMGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLALPVLCLLANTTFPCSQPPCTPCCYEKEPESTLRMLEDNVMRPGYYQLLKASLTCRQRRLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNANFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKAATYYEAAAYLWNEQQPLFWLQALIPLAALIVLCNCLKLLPCCCKTLAFLAVMSIGAHTVSAYEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 191 - DNA, 4362 bp, synthetic construct
  misc_feature 1..4362: note = Synthetic
  source 1..4362: mol_type = other DNA
ttagtgcctgctaaacgacacgcatagcaccacaattaaaattaaggcagcaacagcaacaattaatcctactcctcccgtaatcttctgcacccaagacattgccgttgtggatatatcctggaccccaagggtggtgtgtgatgctgggtaattgactatgtggtcctttggagggtggcatgcggctgcgcagtgtacttgtgtggagcacacttgcacgcgaaactcggcgcttgccagggctgttgagaaggatatttgcagctgggagttcccctctacttctacgtcggcttctcgaatggtaacggcgttggtcatcgaatgtactgcacatttacctttcttgctagctgtgtatttgatgatggcgacgcccccaaagtcggaggagtgagtgcaggctggtacttcgcatgacatgtccgttacagagggtgcatcgacaaccctagtaaaggccgcatccggtatgtcgatggaaattggtatgttccccacagcgcaatttacagctcttaccgggtttgtcgcaatctggcaaccgaacggtgccgtgtgctgtagcgatgctcctcgttccttcagccaatacttgaagccagatggtgcctgagagtatggtacatgtaccgtgcctgctgctggcctctgtagtaccaactgagtgttggcataaacgtctttactttccggtgtacgactttgaatgtcaccaaattgtcctggtcttcctgcgccaaaaggtgggtagtccatgttgtagacgtcgcctttgtacaccacgattttgttgtcaaaaggtgtccaggcggaggacattgggcccacgacaaacttggcgtcctttactgtgacggcatggtcaccgttagcgtaggcagctacggtaatgttgtttccttggtaaaggacgcggagcttcgccgacgccgatgcggtgtgggctctgtaggccgatgcaaactctgttttgcaagattcagatttctctacatgtgcctcgctcaattgcgtattttcggcgtcgcaaaagcagtaggcgccgccccacataaatgggtagactccagtaaagaccttgcagctgtagtctggtaggctcttgtccttgcactctgctgtaccacagcacttcacgtacggggaggggatgacagttttgtactcgcacgtgatgtagtcaagtgacagtgttggttccaaggtgactgattgtagctccatctccaacaccatggggctgtaacccggtctgttgacaagagtcttatacggtactcccaccgtgttcgggatcactgttacgtgttcgtacgcgctcacagtgtgggcaccgatgctcattacggctaaaaaagccagggtcttacagcagcatggcaagagtttcagacagttgcacaggacgatcaaggcggccagcgggataagagcctgcaaccagaacaggggctgctgttcgttccatagatatgccgcagcctcgtaatatgtggccgccttggtcgttctgacgcagcatagcaggctgagcaggaagggaacagtggctcctggtgttaattcatatggtgtaatgcatctgcgccgtgcgcacacacacattcccactgctgtgcccaccatcgacagaagcacgaacgaggccaccgacacaatgactacagtcatagtggggtacagctcataatagtacaagattatctcatgtgggtgaccatgagcagtaccgttcgtagacatctgcggccagtacttgtatggttcgttgttgccccaagtgacctccagaccctcagtaggcacggtcaaggtaacctccttcttgtgtgtcacccactcctcgtggtaatttggttcctgtcccatgttacggtaagacaagagtgtcggatggtcaggatacagcagcatggtgacttggttttttccgtaagttactgtagggtttcttgcttttggcactctgcaagtcacgtttgccaatgggaatgggatgtggatctttcctttacggtccccgagttcagcgttgcgcgggactaaaggggagttgtattgccaattcttgtgattagtgactgcagcatggcactgatcaattttgcagttattgatcactttgtctgtggttgtcagtccctcgtttgagccaccgcagttgcacttgtaccgcaccgtctgcccattaactgtgatcttcacgttgccagactgctgcgtcatcagcgtgcggtcaggagtatctgggggcatatgcacctctatctcctcagcagtggcagcggtgctctgcacgtacgtgctgcaaggtaactctttaccatgttgtggtcgagagtggaacctctccctacctatcacaggtggttcatgatggaacgggtgtgtgcatgtgtggctgatctttctgctgtccgtaaatcccactgtcagcgtctctcctttcgggcatcgggcgagaataaagtgtcccatggtcccggtgatcgtgcacggtgctgaagtccttacaagcaatccggctcgctccgcgtccgctggcgtatggctatccatatagcgcagcttggtccaatcgtggctgtcatctgtctttatcccgatctgcaaagagacctggattttcagcgttccgtccgttgcttcatttctgatgcgctccaatgcgatagggctgtggcacgaatgcccttctccgcagtcaggacaatgagctagatatggtcttgtggctttatagacattaaaattggcgttccgctccatggcgaaggcgtaccggggcaccaccaggttgccggtggcctcgaaggtgatcttgtcgccgggctccaccagggtccagtagtagttcatccggccctcctggtcccgcaccttgggccggatggcgatctcgggcttgaacttcttgctgtaccggctgctgcccacgaacacgtaggtgtcggcgttctggtacaggctctgctggtcggcgctggtgctggggtggtggatgccccacagcaccagcacctccttgcccttgtcgttgatgtagctcttgctcagcttggggtagctgttgcccttcttcaccagccagatcaggttcttgtagaagctcttggcgccggcgtgggggcaggcggcggtcacgcccttgttgctgtcgtggttgggccagctgctggtcttggggaagatctcgaaccgctcgaagctgctcacgctgctcagctgctcccgcagctcctcgtagtcgatgaagtcgccggggaagcaggtgccgttgtcgctgctgggggtctccacgatgtagctccagctgctggcggtgctcaggctctcgcactcggggttgcccagaatccagccggcgatgttgcacttgcccaggtgcaggcgtctttggcggcaagtcagcgatgcttttagtagctggtagtatccgggtctcatcacgttgtcctcaagcatgcgcaaggtgctttccggttccttttcgtagcagcagggtgtgcaaggcggctgagagcaggggaatgtagtgtttgccaacaggcacaagaccgggagggcgaggctccactcttcggctccctcaggggtaatttttgtgacgatgtctttgttccacgtcaccacggagagggccgtgcgggcaccttcgttggcccctcctaggacgatggccaccacccgtcctttgttgtcgaagatcggtctgccgctgtctcccggcttgcctgcacccgtcgggatagtgaaccggcctcctgaatactgcactgctccgtgatgccagttatagtacccctcgggtttctcgtgggtaaacttcgaggcatcagacttcatgtgcaccggtatctgtgcacattcaagatcgtatttagacgaccgcttaaaggccagtttagccagatcggcattgtcgatagttcccttcacatgtgctggtttcattactttatcccccaccaggcatgcgtagcccatcactttgccttcatgcttgacttcgaagatgcaatcattttcaattttcatgcacattctctccctacggcctggtttcttcttcttttgagccggcttcttttgtggtggttgcttcttttgctttgggtcgttttgcggcgcctgcttcttctgcctttgcttcttgtttttccgatttctgcgaggcttctgttgaggtaccgcgcgcatggtcaatttgttgactgcggagatcagctgggcgagttgcccagcctgcctctgtggacgtggtctaggtctaattacttgaattgtagggcgtggggcccagggtcggggttggtaccttctgttatagaaagtttgcgtcgggatgaactccat

(19) United States
(12) Patent Application Publication
(10) Pub. No.: US 2024/0226012 A1
(43) Pub. Date: Jul. 11, 2024

(54) NOVEL MULTIVALENT NANOPARTICLE-BASED VACCINES

(71) Applicant: The USA, as represented by the Secretary, Department of Health and Human Services, Bethesda, MD (US)

(72) Inventors: Barney S. Graham, Smyrna, GA (US); Masaru Kanekiyo, Chevy Chase, MD (US); Hadi M. Yassine, Boyds, MD (US)

(73) Assignee: The USA, as represented by the Secretary, Department of Health and Human Services, Bethesda, MD (US)

(21) Appl. No.: 18/615,895

(22) Filed: Mar. 25, 2024

Related U.S. Application Data
(62) Division of application No. 17/519,142, filed on Nov. 4, 2021, now Pat. No. 11,938,221.
Application No. 17/519,142 is a division of application No. 15/540,898, filed on Jun. 29, 2017, now Pat. No. 11,191,727.
(60) Provisional application No. 62/098,755, filed on Dec. 31, 2014.

(51) Int. Cl. A61K 9/16; A61K 39/00; A61K 39/12; A61K 39/145; C12N 7/00

(52) U.S. Cl. CPC A61K 9/167; A61K 39/12; A61K 39/145; C12N 7/00; A61K 2039/575; A61K 2039/6031; A61K 2039/6068; A61K 2039/6075; A61K 2039/70; C12N 2760/16134; C12N 2760/16171; Y02A 50/30

(57) ABSTRACT

Novel, nanoparticle-based vaccines are provided that elicit an immune response to a broad range of infectious agents, such as influenza viruses. The nanoparticles comprise a heterogeneous population of fusion proteins, each comprising a monomeric subunit of a self-assembly protein, such as ferritin, joined to one or more immunogenic portions of a protein from an infectious agent, such as influenza virus. The fusion proteins self-assemble to form nanoparticles that display a heterogeneous population of immunogenic portions on their surface. When administered to an individual, such nanoparticles elicit an immune response to different strains, types, subtypes and species with in the same taxonomic family. Thus, such nanoparticles can be used to vaccinate an individual against infection by different Types, subtypes and/or strains of infectious agents. Also provided are specific fusion proteins, nucleic acid molecules encoding such fusion proteins and methods of using nanoparticles of the invention to vaccinate individuals.

### CROSS-REFERENCE TO RELATED APPLICATIONS

This is a divisional of U.S. Non-Provisional patent application Ser. No. 17/519,142, filed on Nov. 4, 2021, which is a divisional of U.S. Non-Provisional patent application Ser. No. 15/540,898, filed on Jun. 29, 2017, now U.S. Pat. No. 11,191,727, which is the U.S. National Stage of International Application No. PCT/US2015/068272, filed on Dec. 31, 2015, which was published in English under PCT Article 21(2), which in turn claims the benefit of U.S. Provisional Application No. 62/098,755, filed on Dec. 31, 2014. Each of the above-listed applications is incorporated by reference herein in its entirety.

### REFERENCE TO SEQUENCE LISTING

This application contains a Sequence Listing submitted as an XML file in the form of the filed name “4239-104866-21_Sequence.xml” (360,448 bytes), which was created on Mar. 25, 2024, which is incorporated by reference herein.

### SUMMARY OF THE INVENTION

The present invention provides novel, nanoparticle-based vaccines that are easily manufactured, potent, and which elicit broadly neutralizing antibodies against infectious agents, such as influenza virus, HIV and human papilloma virus. In particular, the present invention provides novel nanoparticles (nps), the surfaces of which display a heterogeneous population of immunogenic portions of proteins from infectious agents. Such nanoparticles comprise fusion proteins, each of which comprises a monomeric subunit of ferritin joined to one or more immunogenic portions of proteins from infectious agents. When such nanoparticles are administered to an individual, they elicit an immune response to proteins from a broad range of infectious agents.

In one embodiment, the invention is a nanoparticle comprising fusion proteins, wherein the surface of the nanoparticle displays immunogenic portions of corresponding proteins from at least two infectious agents, wherein the at least two infectious agents are from different corresponding taxonomic groups within the same taxonomic family. In certain aspects of the invention, the fusion proteins comprise at least a portion of a self-assembling, monomeric subunit joined to at least one immunogenic portion of a protein from an infectious agent.

In one embodiment, the invention is a nanoparticle comprising at least a first fusion protein and a second fusion protein, each fusion protein comprising at least a portion of a self-assembling, monomeric subunit joined to at least one immunogenic portion of a protein from an infectious agent, wherein the immunogenic portion of the first fusion protein is from a protein from a first infectious agent; wherein the immunogenic portion of the second fusion protein is from a protein from a second infectious agent; wherein the proteins from the first and second infectious agents are corresponding proteins; and wherein the first and second infectious agents are from different corresponding taxonomic groups within the same taxonomic family.

In the above embodiments, the corresponding taxonomic groups can be genera, types, subtypes, species or strains. In certain aspects, the monomeric subunit can be a monomeric ferritin subunit protein, a monomeric encapsulin protein, a monomeric 03-33 protein, a monomeric SOR protein, a monomeric LS protein, a monomeric PDC protein or a Chikungunya virus structural polyprotein. In certain aspects the infectious agents are viruses. In certain aspects, the infectious agents can be, for example, influenza viruses, human immunodeficiency viruses (HIV), flaviviruses (e.g., hepatitis virus, dengue virus, etc.), human papillomaviruses (HPV), rhinoviruses, coronaviruses, enteroviruses, polyomaviruses, respiratory synctial viruses (RSV), human metapneumoviruses, ebola viruses, Marburg viruses, alphaviruses (e.g., Chikungunya virus, Ross River virus, Semliki Forest virus, Sindbis virus, Mayaro virus, etc), Porcine Epidemic Diarrhea, Porcine reproductive and respiratory syndrome virus and foot and mouth disease virus.

In one embodiment, nanoparticles of the above embodiments can be produced by introducing into a cell one or more nucleic acid molecules encoding fusion proteins comprising at least a portion of a self-assembling, monomeric subunit joined to at least one immunogenic portion of a protein from an infectious agent, and incubating the cell under conditions suitable for expression of the encoded proteins to form nanoparticles. In certain embodiments, such a method can comprise further purification and/or isolation of the nanoparticles.

In one embodiment of the invention, nanoparticles of the embodiments listed above are used to prepare a medicament for protecting an individual from an infectious agent. In such embodiments, the nanoparticles comprise immunogenic portions of proteins from infectious agents in the same taxonomic family as the infectious agent against which the individual is being protected. In certain embodiments, the medicament is used to vaccinate the individual.

One embodiment of the invention is a method to elicit a protective immune response against an infectious agent, the method comprising administering to an individual a nanoparticle of the embodiments of the invention, or a composition or medicament comprising embodiments of the invention, wherein the nanoparticles comprise immunogenic portions of proteins from infectious agents in the same taxonomic family as the infectious agent against which the protective immune response is being elicited.

One embodiment of the invention is a method to elicit neutralizing antibodies against an infectious agent, the method comprising administering to an individual a nanoparticle of the embodiments of the invention, or a composition or medicament comprising embodiments of the invention, wherein the nanoparticles comprise immunogenic portions of proteins from infectious agents in the same taxonomic family as the infectious agent against which the neutralizing antibodies are desired.

In one embodiment, the invention is a nanoparticle that comprises self-assembling fusion proteins, and in this embodiment the nanoparticle displays on its surface a heterogeneous population of immunogenic portions from HA proteins from one or more Type, Group, subtype and/or strain of influenza virus.

In another embodiment, the invention is a nanoparticle that comprises a heterogeneous population of fusion proteins, and in this embodiment each fusion protein comprises at least a portion of a monomeric subunit protein capable of self-assembling into a nanoparticle joined to at least one immunogenic portion from an influenza virus hemagglutinin protein, such that the heterogeneous population comprises at least two different species of fusion proteins, and such that the difference between two species of fusion proteins is due, at least in part, to sequence differences in the immunogenic portion from an influenza virus HA protein.

In yet another embodiment, the invention is a nanoparticle that comprises at least two species of fusion proteins, and in this embodiment each fusion protein comprises at least a portion of a monomeric subunit protein capable of self-assembling into a nanoparticle joined to at least one immunogenic portion from an influenza virus HA protein, such that the species of fusion protein differ from one another due, at least in part, to differences in the sequences of the immunogenic portion from an influenza virus hemagglutinin protein.

In still another embodiment, the invention is a nanoparticle that comprises at least a first species of fusion protein and a second species of fusion protein, and in this embodiment the fusion proteins comprise at least a portion of a monomeric subunit protein capable of self-assembling into a nanoparticle joined to at least one immunogenic portion from an influenza virus hemagglutinin protein, such that the species of fusion proteins differ from one another due, at least in part, to differences in the sequences of the immunogenic portion from an influenza virus hemagglutinin protein.

In the above embodiments, the different species of fusion proteins contain immunogenic portions from HA proteins of influenza viruses in different taxonomic groups within the orthomyxoviridae family.

In the above embodiments, ferritin-based nanoparticle can form an octahedron, which can consist of 24 subunits. Further, the immunogenic portions of the influenza HA proteins can be displayed on the surface of the nanoparticle with a spacing range in the range of about 50 Å to about 100 Å. Additionally, the monomeric subunit protein can be selected from a monomeric ferritin subunit protein, a monomeric encapsulin protein, a monomeric 03-33 protein, a monomeric SOR protein, a monomeric LS protein, a monomeric PDC protein and Chikungunya virus envelope protein. The monomeric ferritin subunit protein can be selected from the a bacterial ferritin, a plant ferritin, an algal ferritin, an insect ferritin, a fungal ferritin and a mammalian ferritin and in preferred embodiments, is selected from a monomeric subunit of a Helicobacter pylori ferritin protein, a monomeric subunit of a Escherichia coli ferritin protein and a monomeric subunit of a bullfrog ferritin protein. In still another preferred embodiment, the monomeric ferritin subunit protein can be a hybrid protein that comprises at least a portion of a bullfrog ferritin protein joined to at least a portion of a ferritin protein selected from a Helicobacter pylori ferritin protein and an Escherichia coli ferritin protein.

In one aspect of the embodiments of the invention, the monomeric subunit protein can comprise at least 25 contiguous amino acids from a protein selected from a monomeric ferritin subunit protein, a monomeric encapsulin protein, a monomeric 03-33 protein, a monomeric SOR protein, a monomeric LS protein, a monomeric PDC protein and Chikungunya virus envelope protein.

In still another aspect of the embodiments of the invention, the monomeric subunit protein can comprise at least 25 contiguous amino acids from an amino acid sequence selected from a sequence selected from SEQ ID NO:64, SEQ ID NO:67, SEQ ID NO:70, SEQ ID NO:73, SEQ ID NO:76, SEQ ID NO: 79, SEQ ID NO:82, SEQ ID NO:85, SEQ ID NO:88, SEQ ID NO:91 and SEQ ID NO:94. Alternatively, the monomeric subunit protein can comprise an amino acid sequence at least about 80% identical to an amino acid sequence selected from SEQ ID NO:64, SEQ ID NO:67, SEQ ID NO:70, SEQ ID NO:73, SEQ ID NO:76, SEQ ID NO:79, SEQ ID NO:82, SEQ ID NO:85, SEQ ID NO:88, SEQ ID NO:91 and SEQ ID NO:94. Also, the monomeric subunit protein can comprise an amino acid sequence selected from SEQ ID NO:64, SEQ ID NO:67, SEQ ID NO:70, SEQ ID NO:73, SEQ ID NO:76, SEQ ID NO:79, SEQ ID NO:82, SEQ ID NO: 85, SEQ ID NO: 88, SEQ ID NO:91 and SEQ ID NO:94.

In one aspect of the embodiments of the invention, the HA protein can be from a virus selected from A/New Caledonia/20/1999 (H1N1), A/California/04/2009 (H1N1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 (HIN1), A/Malaysia/54 (H1N1), A/Iowa/43 (H1N1), A/Hong Kong/117/77 (HIN1), A/Fort Monmouth/1/47 (H1N1), A/Brisbane/59/07 (H1N1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2).

In yet another aspect of the embodiments of the invention, the HA protein can comprise at least 25 contiguous amino acids from the hemagglutinin protein of an influenza virus selected from the group consisting of A/New Caledonia/20/1999 (H1N1), A/California/04/2009 (H1N1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 (H1N1), A/Malaysia/54 (H1N1), A/Iowa/43 (H1N1), A/Hong Kong/117/77 (H1N1), A/Fort Monmouth/1/47 (H1N1), A/Brisbane/59/07 (HIN1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2).

In yet another aspect of the embodiments of the invention, the HA protein can comprise at least 25 contiguous amino acids from a sequence selected from SEQ ID NOs: 1-62. The HA protein can comprise an amino acid sequence at least about 80% identical to an amino acid sequence selected from SEQ ID NOs: 1-62. Also, the hemagglutinin protein can comprise an amino acid sequence selected from SEQ ID NOs: 1-62.

In still another aspect of the embodiments of the invention, the HA protein can be capable of eliciting an immune response to a protein comprising an amino acid sequence selected from SEQ ID NOs: 1-62.

In another aspect of the embodiments of the invention, the immunogenic portion can comprise the receptor-binding domain of an influenza HA protein. Further, the immunogenic portion can be selected from amino acid residues 56-264 of a sequence selected from SEQ ID NOs: 1-62.

In yet another aspect of the embodiments of the invention, the at least two species of fusion proteins can comprise immunogenic portions obtained from HA proteins from two different strains of influenza virus. Also, the at least two species of fusion proteins can comprise immunogenic portions obtained from HA protein from two different subtypes of influenza virus.

In still another aspect of the embodiments of the invention, at least one species of fusion protein can comprise a linker sequence.

In another aspect of the embodiments of the invention, the nanoparticle can elicit an immune response against the RBD region of an influenza HA protein. In one aspect, the nanoparticle can elicit an immune response to an influenza virus strain that is heterologous to the strains of influenza viruses from which the HA immunogenic portions were obtained. In still another aspect, the nanoparticle can elicit an immune response to an influenza virus that is antigenically divergent from the influenza virus from which the hemagglutinin proteins were obtained.

In still another aspect of the embodiments of the invention, the heterogeneous population can comprise between 2 and 60 species of fusion proteins. In still another aspect of the embodiments of the invention, the heterogeneous population can comprise between 2 and 240 species of fusion proteins.

Another embodiment of the present invention is a fusion protein comprising an amino acid sequence at least 80% identical to a sequence selected from SEQ ID NO:97, SEQ ID NO:100, SEQ ID NO: 103, SEQ ID NO: 106, SEQ ID NO: 109, SEQ ID NO: 112, SEQ ID NO:115, SEQ ID NO: 118, SEQ ID NO: 121, SEQ ID NO: 124, SEQ ID NO: 127, SEQ ID NO:130, SEQ ID NO: 133, SEQ ID NO: 136, SEQ ID NO:139, SEQ ID NO: 142, SEQ ID NO:145, SEQ ID NO:148, SEQ ID NO: 151, SEQ ID NO: 154, SEQ ID NO: 157, SEQ ID NO:160, SEQ ID NO:163, SEQ ID NO: 166, SEQ ID NO:169, SEQ ID NO: 172, SEQ ID NO:175, SEQ ID NO:178, SEQ ID NO: 181, SEQ ID NO: 184, SEQ ID NO: 187 and SEQ ID NO:190. The fusion protein can also comprise an amino acid sequence selected from SEQ ID NO:97, SEQ ID NO: 100, SEQ ID NO: 103, SEQ ID NO: 106, SEQ ID NO: 109, SEQ ID NO: 112, SEQ ID NO:115, SEQ ID NO:118, SEQ ID NO: 121, SEQ ID NO:124, SEQ ID NO:127, SEQ ID NO: 130, SEQ ID NO: 133, SEQ ID NO:136, SEQ ID NO:139, SEQ ID NO: 142, SEQ ID NO:145, SEQ ID NO: 148, SEQ ID NO: 151, SEQ ID NO: 154, SEQ ID NO: 157, SEQ ID NO:160, SEQ ID NO: 163, SEQ ID NO: 166, SEQ ID NO: 169, SEQ ID NO: 172, SEQ ID NO:175, SEQ ID NO: 178, SEQ ID NO: 181, SEQ ID NO: 184, SEQ ID NO: 187 and SEQ ID NO:190.

A further embodiment is a nucleic acid molecule encoding any of the fusion proteins described above. In this embodiment, the nucleic acid sequence can be at least 80% identical to a sequence selected from SEQ ID NO:96, SEQ ID NO:99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO:123, SEQ ID NO: 126, SEQ ID NO:129, SEQ ID NO: 132, SEQ ID NO: 135, SEQ ID NO:138, SEQ ID NO:141, SEQ ID NO:144, SEQ ID NO: 147, SEQ ID NO: 150, SEQ ID NO:153, SEQ ID NO:156, SEQ ID NO: 159, SEQ ID NO: 162, SEQ ID NO: 165, SEQ ID NO:168, SEQ ID NO:171, SEQ ID NO:174, SEQ ID NO: 177, SEQ ID NO: 180, SEQ ID NO:183, SEQ ID NO:186 and SEQ ID NO:189. In still another aspect, the nucleic acid sequence can comprise a sequence selected from SEQ ID NO:96, SEQ ID NO:99, SEQ ID NO: 102, SEQ ID NO: 105, SEQ ID NO: 108, SEQ ID NO: 111, SEQ ID NO: 114, SEQ ID NO:117, SEQ ID NO:120, SEQ ID NO: 123, SEQ ID NO: 126, SEQ ID NO: 129, SEQ ID NO:132, SEQ ID NO: 135, SEQ ID NO: 138, SEQ ID NO: 141, SEQ ID NO: 144, SEQ ID NO:147, SEQ ID NO:150, SEQ ID NO: 153, SEQ ID NO: 156, SEQ ID NO: 159, SEQ ID NO:162, SEQ ID NO: 165, SEQ ID NO: 168, SEQ ID NO: 171, SEQ ID NO: 174, SEQ ID NO:177, SEQ ID NO: 180, SEQ ID NO: 183, SEQ ID NO: 186 and SEQ ID NO: 189. Further in this embodiment, a plasmid can comprise the nucleic acid molecule of any of the nucleic acid molecules described above.

Another embodiment of the present invention is a method for producing a nanoparticle of any of the nanoparticles described above, the method comprising introducing one or more nucleic acid molecules encoding fusion proteins, wherein each fusion protein can comprise at least a portion of a monomeric subunit protein capable of self-assembling into a nanoparticle joined to at least one immunogenic portion from an influenza virus hemagglutinin protein; and incubating the cell under conditions suitable for expressing the encoded proteins and forming nanoparticles. A further aspect of this embodiment can comprise isolating the nanoparticles from the cell.

Another embodiment of the present invention is a method of eliciting an immune response against influenza virus, the method comprising administering to an individual a nanoparticle as described above.

Another embodiment of the present invention is a method of vaccinating an individual against influenza virus, such that the method can comprise administering to the individual a nanoparticle as described above. Accordingly, another embodiment of the present invention is an immunogenic composition comprising a nanoparticle of the invention. Another embodiment of the invention is a medicament for use in vaccinating an individual, or electing an immune response, against influenza virus, the medicament comprising a nanoparticle of the present invention.

A further embodiment of the present invention is a kit. The kit can comprise a nanoparticle as described above, compositions and medicaments comprising such nanoparticles, a fusion protein and/or a nucleic acid molecule as described above.

### BACKGROUND

Protective immune responses induced by vaccination against influenza virus are primarily directed to the viral hemagglutinin (HA) protein, which is a glycoprotein on the surface of the virus responsible for interaction of the virus with host cell receptors. HA proteins on the virus surface are trimers of hemagglutinin protein monomers that are enzymatically cleaved to yield amino-terminal HA1 and carboxy-terminal HA2 polypeptides. The globular head consists exclusively of the major portion of the HA1 polypeptide, whereas the stem that anchors the hemagglutinin protein into the viral lipid envelope is comprised of HA2 and part of HA1. The globular head of a hemagglutinin protein includes two domains: the receptor binding domain (RBD), an ˜148-amino acid residue domain that includes the sialic acid-binding site, and the vestigial esterase domain, a smaller ˜75-amino acid residue region just below the RBD. The top part of the RBD adjacent to the 2,6-sialic acid recognition sites includes a large region (amino acids 131-143, 170-182, 205-215 and 257-262, 1918 numbering) (referred to herein as the RBD-A region) of over 6000 Å² per trimer that is 95% conserved between A/South Carolina/1/1918 (1918 SC) and A/California/04/2009 (2009 CA) pandemic strains. The globular head includes several antigenic sites that include immunodominant epitopes. Examples include the Sa, Sb, Ca₁, Ca₂ and Cb antigenic sites (see, for example, Caton A J et al, 1982, Cell 31, 417-427). The RBD-A region includes the Sa antigenic site and part of the Sb antigenic site.

Antibodies against influenza often target variable antigenic sites in the globular head of HA, which surround a conserved sialic acid binding site, and thus, neutralize only antigenically closely related viruses. The variability of the HA head is due to the constant antigenic drift of influenza viruses and is responsible for seasonal endemics of influenza. In contrast, gene segments of the viral genome can undergo reassortment (antigenic shift) in host species, creating new viruses with altered antigenicity that are capable of becoming pandemics [Salomon, R. et al. Cell 136, 402-410 (2009)]. Until now, each year, influenza vaccine is updated to reflect the predicted HA and neuraminidase (NA) for upcoming circulating viruses.

Current vaccine strategies for influenza use either a chemically inactivated or a live attenuated influenza virus. Both vaccines are generally produced in embryonated eggs which present major manufacturing limitations due to the time consuming process and limited production capacity. Another more critical limitation of current vaccines is its highly strain-specific efficacy. These challenges became glaring obvious during emergence of the 2009 HIN1 pandemic, thus validating the necessity for new vaccine platforms capable of overcoming these limitations. Virus-like particles represent one of such alternative approaches and are currently being evaluated in clinical trials [Roldao, A. et al. Expert Rev Vaccines 9, 1149-1176 (2010); Sheridan, C. Nat Biotechnol 27, 489-491 (2009)]. Instead of embryonated eggs, VLPs comprising HA, NA and matrix protein 1 (M1) can be mass-produced in mammalian or insect cell expression systems [Haynes, J. R. Expert Rev Vaccines 8, 435-445 (2009)]. The advantages of this approach are its particulate, multivalent nature and the authentic display of properly folded HA proteins that faithfully mimic the infectious virion. In contrast, by the nature of its assembly, the enveloped VLPs contain a small but finite host cell component that may present potential safety, immunogenicity challenges following repeated use of this platform [Wu, C. Y. et al. PLOS One 5, c9784 (2010)]. Moreover, the immunity induced by the VLPs is essentially the same as the immunity induced by current vaccines, and thus, does not likely improve both potency and breadth of vaccine-induced protective immunity. In addition to VLPs, a recombinant HA protein has also been evaluated in humans [Treanor, J. J. et al. Vaccine 19, 1732-1737 (2001); Treanor, J. J. JAMA 297, 1577-1582 (2007)], though the ability to induce protective neutralizing antibody titers are limited. The recombinant HA proteins used in those trials were produced in insect cells and might not form native trimer preferentially [Stevens, J. Science 303, 1866-1870 (2004)].

Recently, entirely new classes of broadly neutralizing antibodies against influenza viruses were isolated. One class of antibodies recognizes the highly conserved HA stem [Corti, D. et al. J Clin Invest 120, 1663-1673 (2010); Ekiert, D. C. et al. Science 324, 246-251 (2009); Kashyap, A. K. et al. Proc Natl Acad Sci USA 105, 5986-5991 (2008); Okuno, Y. et al. J Virol 67, 2552-2558 (1993); Sui, J. et al. Nat Struct Mol Biol 16, 265-273 (2009); Ekiert, D. C. et al. Science 333, 843-850 (2011); Corti, D. et al. Science 333, 850-856 (2011)], and another class of antibodies precisely recognizes the sialic acid binding site of the RBD on the variable HA head [Whittle, J. R. et al. Proc Natl Acad Sci USA 108, 14216-14221 (2011); Krause, J. C. et al. J Virol 85, 10905-10908 (2011)]. Unlike strain-specific antibodies, those antibodies are capable of neutralizing multiple antigenically distinct viruses, and hence inducing such antibodies has been a focus of next generation universal vaccine [Nabel, G. J. et al. Nat Med 16, 1389-1391 (2010)]. However, robustly eliciting these antibodies with such heterologous neutralizing profile by vaccination has been difficult [Steel, J. et al. MBio 1, e0018 (2010); Wang, T. T. et al. PLOS Pathog 6, e1000796 (2010); Wei, C. J. et al. Science 329, 1060-1064 (2010)].

Despite several alternatives to conventional influenza vaccines, advances in biotechnology in past decades have allowed engineering of biological materials to be exploited for the generation of novel vaccine platforms. Ferritin, an iron storage protein found in almost all living organisms, is an example which has been extensively studied and engineered for a number of potential biochemical/biomedical purposes [Iwahori, K. U.S. Patent 2009/0233377 (2009); Meldrum, F. C. et al. Science 257, 522-523 (1992); Naitou, M. et al. U.S. Patent Publication No. 2011/0038025 (2011); Yamashita, I. Biochim Biophys Acta 1800, 846-857 (2010)], including a potential vaccine platform for displaying exogenous epitope peptides [Carter, D. C. et al. U.S. Patent Publication No. 2006/0251679 (2006); Li, C. Q. et al. Industrial Biotechnol 2, 143-147 (2006)]. Its use as a vaccine platform is particularly interesting because of its self-assembly and multivalent presentation of antigen which induces stronger B cell responses than monovalent form as well as induce T-cell independent antibody responses [Bachmann, M. F. et al. Annu Rev Immunol 15, 235-270 (1997); Dintzis, H. M. et al. Proc Natl Acad Sci USA 73, 3671-3675 (1976)]. Further, the molecular architecture of ferritin, which consists of 24 subunits assembling into an octahedral cage with 432 symmetry has the potential to display multimeric antigens on its surface.

There remains a need for an efficacious influenza vaccine that provides robust protection against influenza virus. There particularly remains a need for an influenza vaccine that elicits broadly a neutralizing immune response, thereby protecting individuals from heterologous strains of influenza virus, including evolving seasonal and pandemic influenza virus strains of the future. The present invention meets this need by providing a novel, multivalent nanoparticle-based, influenza vaccine that is easily manufactured, potent, and elicits broadly neutralizing influenza antibodies.

### BRIEF DESCRIPTION OF THE FIGURES

FIG. 1. Theoretical model of immune triggering by a supernatural heterogeneous antigen array on particulate immunogen. (Left Images) Immune triggering upon natural homogeneous antigen array. Particulate homogeneous antigens are built by an antigenically identical subunit and thereby displaying antigenically homogeneous antigens to immune system. B cells harboring B cell receptor (BCR) specific to an antigen displayed on the homogeneous array are stimulated strongly upon encountering the “matched” antigenic stimulation (left middle). B cells harboring more BCR with broader specificity are also stimulated by the homogeneous antigen array but lesser extent presumably the binding affinity of broader specific BCR to the antigen is not as tight as narrower specific BCR to the “matched” antigen (left bottom). The weaker affinity of broader specific BCR to antigen may partly be due to its cross-reactivity because the BCR recognizes other antigenically distinct antigens and thus avoids contacting antigenically heterogeneous parts on antigens (smaller antibody footprint on antigens). (Right Images) Immune triggering upon supernatural heterogeneous antigen array. Particulate heterogeneous antigens are synthetically built using antigenically heterogeneous subunits, which display antigenically heterogeneous antigens to immune system. Upon stimulation with the heterogeneous antigen array, BCRs having narrow specificity only recognize a subset of antigens on the particulate antigen and thus are not stimulated by this antigen (middle right); BCRs having broader specificity recognize larger numbers of antigens on the particulate antigen and thus are stimulated by this antigen (bottom right). In this situation, the B cells harboring BCR with broader specificity have a better chance to outcompete B cells harboring BCR with narrower specificity, therefore selecting cross-reactive B cells otherwise being overcast by others.

FIG. 2. Schematic representation of HA RBD-ferritin single polypeptide design. HA RBD-ferritin construct without Furin-2A (F2A) self cleavage module (top). Two or three HA RBD-ferritin constructs are connected with F2A self cleavage module (middle or bottom, respectively). As the fusion proteins are produced in producer cells, the cellular protease furin cleaves its cleavage site at the N-terminus of F2A module, and 2A protease cleaves the second (and third) HA RBD-ferritin from the F2A module. As the result, equimolar amount of each HA-RBD-ferritin is produced.

FIGS. 3A-3C. Electron microscopic analysis of HA RBD-nanoparticles. (A) Negative stain electron micrographs of NC99 RBD-nanoparticles; (B) Negative stain electron micrographs of CA09 RBD-nanoparticles; (C) Negative stain electron micrographs of co-assembled (CoAsmbl 2) RBD-nanoparticles. Purified particles were adsorbed to freshly glow-discharged carbon-coated grids and stained with uranyl formate.

FIG. 4. Two-dimension classifications of NC99 RBD-nanoparticles were calculated using images stained with ammonium molybdate instead of uranyl formate.

FIG. 5. Characterization of HA RBD-nanoparticles. Monovalent (NC99=A/New Caledonia/20/1999 and CA09 A/California/04/2009) and co-assembled (CoAsmbl2=A/New Caledonia/20/99 (NC99)+A/California/04/09 (CA09)) nanoparticles were immunoprecipitated using either anti-NC99 (3u-u)(left), anti-pandemic HIN1 HA (2D1)(center) or anti-HA stem (C179) (right) monoclonal antibodies. The precipitated material was then analyzed by SDS-PAGE Protein bands at ˜150 and ˜50 kDa correspond to IgG, RBD-nanoparticle subunits, respectively.

FIG. 6. SDS-PAGE analysis of purified HA RBD-nanoparticles from different HIN1 strains and co-assembled RBD-nanoparticles with different combinations of HA. NC99=A/New Caledonia/20/1999; CA09=A/California/04/2009; WS33=A/Wilson-Smith/1933; AB48=A/Albany/4835/1948; BR07=A/Brisbane/59/2007; IA43=A/Iowa/1943; HK77=A/Hong Kong/117/1977; FM47=A/Fort Monmouth/1/1947. CoAsmbl 2=A/New Caledonia/20/99 (NC99)+A/California/04/09 (CA09); CoAsmbl 4=CoAsmbl 2+A/Wilson-Smith/33 (WS33)+A/Albany/4835/48 (AB48); CoAsmbl 6=CoAsmbl 4+A/Brisbane/59/07 (BR07)+A/Iowa/43 (IA43); CoAsmbl 8=CoASmbl 6+A/Hong Kong/117/77 (HK77)+A/Fort Monmouth/1/47 (FM47)

FIG. 7. Hemagglutination inhibitory (HAI) titers against influenza A/New Caledonia/20/1999 virus. (Left panel) Hemagluttination inhibition titers of sera from mice immunized with monovalent nanoparticles (NC99) or mixtures of monovalent nanoparticles (Admix 2, 4 or 6). (Middle panel) Hemagluttination inhibition titers of sera from mice immunized with monovalent nanoparticles (NC99) or multivalent nanoparticles (CoAsmb12, 4, 6 or 8). (Right panel) Side by side comparison, using the data from the left and middle panels, comparing the HAI titers generated by immunizing mice with either admixed monovalent nanoparticles or multivalent nanoparticles displaying corresponding influenza HA proteins. All sera was collected at 2 weeks following the second immunization and tested for hemagluttination inhibition activity. Each dot indicates individual serum sample and is plotted as box-and-whiskers graph. P values were calculated by Student's t-test.

FIG. 8. Neutralization titers against NC99 pseudotyped lentivirus.

(Left panel) Neutralization titer of sera from mice immunized with monovalent nanoparticles (NC99) or mixtures of monovalent nanoparticles (Admix 2, 4 or 6). (Middle panel) Neutralization titer of sera from mice immunized with monovalent nanoparticles (NC99) or multivalent nanoparticles (CoAsmbl2, 4, 6, or 8). (Right panel) Side by Side comparison, using the data from the left and middle panels, comparing the neutralization titers generated by immunizing mice with either admixed monovalent nanoparticles or multivalent nanoparticles displaying corresponding influenza HA proteins. All sera was collected at 2 weeks following the second immunization and tested for hemagluttination inhibition activity. Each dot indicates individual serum sample and is plotted as box-and-whiskers graph. P values were calculated by Student's t-test.

FIG. 9. Neutralization breadth of immune serum. Heatmap representation of HAI titers from mice immunized with either monovalent nanoparticles against NC99 or CA09, admixed monovalent nanoparticles (Admix 4), or multivalent, co-assembled nanoparticles (CoAsmbl 4 or CoAsmbl 8). Each row indicates an individual mouse.

FIG. 10. Detection of HA-specific cross-reactive B cells in peripheral blood cells in HA RBD-nanoparticle-immunized mice. (Upper panels). Gating strategy of mouse whole blood cells. (Bottom panel) FACs analysis using anti-CD3, anti-CD14, anti-CD19, anti-IgD to identify non-naïve B-cell populations in peripheral blood from mice immunized with monovalent nanoparticles against NC99 or CA09, admixed particles (Admix 2, Admix 4 or Admix 6), or multivalent (CoAsmbl 2, CoAsmbl 4, CoAsmbl 6 or CoAsmbl 8). Each dot indicates individual sample.

FIG. 11. Box-and-whiskers plot of FACS data from lower panel in FIG. 10.

FIG. 12. Correlation of NC99/CA09 cross-reactive B cell frequency and antigenic heterogeneity of co-assembled RBD-nanoparticles. X-axis represents antigenic heterogeneity (number of different HA RBD on a single RBD-nanoparticle). Y-axis represents cross-reactive B cell frequency. Pearson correlation was calculated using GraphPad Prism 6.

FIG. 13. Three dimensional reconstruction model of HA trimer in complex with Fab 441D6. (Upper panels) Rotational and top views for reconstructed model of HA:Fab441D6 complex. (Lower panels) Electron microscopy density maps of HA:Fab441D6 complex. Resolution of the final model was ˜18.5 Å.

### DETAILED DESCRIPTION OF THE INVENTION

The present invention relates to a novel, nanoparticle-based, multivalent vaccine that can be used to produce a broadly neutralizing immune response to various infectious agents, such as influenza virus and human immunodeficiency virus (HIV). The present invention builds on previous work showing that monovalent, nanoparticle-based vaccines can be used to induce a protective immune response against a limited number of closely related infectious agents. For example, previous work in the field of influenza vaccines demonstrated that fusion proteins comprising an immunogenic portion of an influenza virus hemagglutinin (HA) protein joined to a self-assembly (SA) protein, to produce an HA-SA fusion protein, will self-assemble into nanoparticles displaying the immunogenic portion of the influenza HA protein on their surface. Moreover, when such nanoparticles are administered to an individual, they elicit a robust, neutralizing immune response to influenza virus. The construction and use of such nanoparticles has been described in U.S. Patent Publication No. 2014-0302079A1, which is incorporated herein by reference in its entirety. Similarly, nanoparticle-based vaccines for Epstein-Barr Virus have been described in International Patent Application No. PCT/US14/60142, which is incorporated herein by reference in its entirety. The present inventors have now discovered that nanoparticles displaying immunogenic portions of proteins from more than one genera, Type, Group, subtype or strain of infectious agent (e.g., influenza virus) can be used as a vaccine to elicit an immune response that neutralizes a variety, including a heterogeneous population, of different, but related, infectious agents. Moreover, the inventors have found that, surprisingly, such multivalent nanoparticles elicit a greater immune response than do vaccines comprising a single species of monovalent nanoparticles, or a mixture of two or more species of monovalent nanoparticles. Thus, a general embodiment of the invention is a nanoparticle made from self-assembling fusion proteins, wherein the surface of the nanoparticle displays a heterogeneous population of immunogenic portions of proteins from two or more infectious agents of the same taxonomic family. In specific embodiments, the two or more infectious agents are divergent enough such that the amino acid sequence of the immunogenic portions of corresponding proteins from the two or more infectious agents differ by at least one amino acid. In certain embodiments, the infectious agents are from different taxonomic groups within the same taxonomic family.

Before the present invention is further described, it is to be understood that this invention is not limited to particular embodiments described herein, as such may, of course, vary. It is also to be understood that the terminology used herein is for the purpose of describing particular embodiments only, and is not intended to be limiting on the finally claimed invention, since the scope of the invention will be limited only by the claims. It should also be understood that while elements of the invention appear in specific locations in the application, the present invention encompasses any combination of the elements disclosed herein.

It must be noted that as used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like, in connection with the recitation of claim elements, or use of a “negative” limitation.

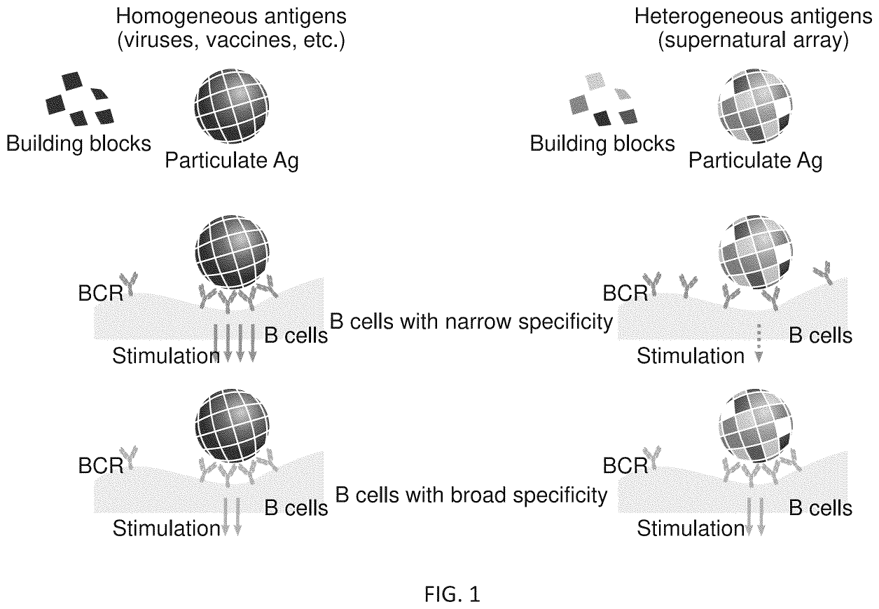

As used herein, a nanoparticle refers to a particle formed from self-assembling, monomeric subunit proteins. For example, ferritin subunit proteins self-assemble into ferritin nanoparticles. Nanoparticles of the present invention are generally spherical, or spheroid, in shape, although other shapes, for example, rod, cube, sheet, oblong, ovoid, and the like, are also useful for practicing the present invention. While nanoparticles of the present invention can vary in size, preferred nanoparticles are those in which the distance between the displayed immunogenic portions of the HA protein globular head region is such that two adjacent immunogenic portions displayed on the nanoparticle can fit the distance of the two antigenic-binding sites of a single B-cell receptor, or about 50-100 Å apart. Such spacing allows each of the two adjacent immunogenic portions to interact with one of the two, identical antigen-binding sites in the same B-cell receptor. Binding of a single B-cell receptor to heterologous immunogenic portions that are adjacent on the surface of the nanoparticle is desirable since it allows for the selection of cross-reactive immune responses. While not intending to be bound by theory, the inventors believe that this is due to the fact that high affinity binding of one antigenic site to an immunogenic portion allows stabilization of low-affinity binding of the other antigenic binding site to a heterologous immunogenic portion. Thus, B-cells are selected that produce cross-reactive antibodies. This concept is illustrated in FIG. 1. It is understood by those skilled in the art that the antigenic binding sites of a B-cell receptor are approximately 50-100 angstroms (Å) apart. Thus, in certain embodiments, the immunogenic portions displayed on the surface of the nanoparticle are separated by about 50-100 Å. In specific embodiment, the immunogenic portions displayed on the surface of the nanoparticle are separated by about 50 Å, by about 60 Å, by about 70 Å, by about 80 Å, by about 90 Å, by about 100 Å. With respect to the spacing of immunogenic portions on the surface of a nanoparticle, the term about refers to a variation of no more than 20%.

According to the present invention, a self-assembling monomeric subunit protein, monomeric subunit protein, self-assembly (SA) protein, self-assembling subunit protein, and the like, of the present invention is a full length, monomeric polypeptide, or any portion or variant thereof, which, is capable of directing self-assembly of monomeric self-assembling subunit proteins into a nanoparticle. Such proteins are known to those skilled in the art. Examples of self-assembly proteins useful for producing nanoparticles of the present invention include, but are not limited to, ferritin, encapsulin, sulfur oxygenase reductase (SOR), lumazine synthase (LS), pyruvate dehydrogenase complex (PDC) dihydrolipoamide acetyltransferase (E2) and the envelope (Env) proteins of alphaviruses such as Chikungunya virus. Representative examples of such proteins are listed below in Table 1.

As used herein, a fusion protein is a recombinant protein containing amino acid sequences from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally found joined together via a peptide bond in their natural environment (e.g., inside a cell). For example, the amino acid sequences of monomeric subunits of ferritin, and the amino acid sequences of influenza hemagglutinin proteins are not normally found joined together via a peptide bond and thus, these two proteins would be considered unrelated. Similarly, the amino acid sequences of monomeric subunits of encapsulin and the amino acid sequences of influenza hemagglutinin proteins or HIV envelope proteins are not normally found joined together via a peptide bond and thus, encapsulin and influenza HA, or encapsulin and HIV envelope protein, would be considered unrelated.

As used herein, a heterogeneous population of immunogenic portions refers a nanoparticle that displays more than one species of immunogenic portion of a protein on its surface. A species of immunogenic portion of a protein of the invention is defined by the specific amino acid sequence of the immunogenic portion. Accordingly, two immunogenic portions having identical amino acid sequences would be considered the same species of immunogenic portion. It should be noted that two fusion proteins comprising the same species of immunogenic portions may or may not vary in regions of amino acid sequences other than the immunogenic portion. If such fusion proteins are identical throughout their entire sequence, they would be considered the same species of fusion protein. Thus, it should be apparent that species of immunogenic portions are defined by variations in their immunogenic portions. Such variation can be due to natural or man-made changes in the amino acid sequence of the immunogenic portion. For example, a new species of immunogenic portion can be made by altering (mutating) the sequence of an existing immunogenic portion through means such as recombinant DNA technology. Methods of making such alterations are known to those skilled in the art.

Alternatively, fusion proteins having different species of immunogenic portions can be made using corresponding proteins, or useful portions thereof, (or nucleic acid molecules encoding such proteins or portions) from unique, but related, infectious agents. For example, it is known that viruses often produce progeny virus having mutations in their envelope (or capsid) protein, the result being that some percentage of the progeny virus avoid detection by the host immune system. Similarly repeated cycles of progeny production result in a heterogeneous population of viruses, with various individual viruses in the population differing in the sequence of their envelope (or capsid) proteins. Such a process eventually results in the production of closely related, but genetically divergent viruses. These divergent viruses are referred to strains, species and subtypes. As these strains, species and subtypes become more divergent, they are further classified into types, generas and/or families. Such classifications can be referred to as taxonomic groups. For examples, a taxonomic group can be a family, a genus, a type, a subtype, a strain or a species. Classification of viruses into various taxonomic groups is well understood by those skilled in the art. With regard to the present invention, preferred nanoparticles are those comprising immunogenic portions from two or more infectious agents within the same family.

As used herein, corresponding proteins are proteins having a similar function in two (or more) different organisms. Corresponding proteins may or may not have identical amino acid sequences, but generally share some sequence homology. In the examples above, the envelope (or capsid) proteins from two closely related viruses are corresponding proteins. As a further example, envelope proteins from different strains of HIV would be considered corresponding proteins, as would hemagglutinin (HA) protein from different strains, subtypes, or genera of influenza virus. In certain embodiments, proteins having the same function in two different infectious agents from the same taxonomic family would be considered corresponding proteins. In certain embodiments, such proteins have at least 50% sequence homology. In certain embodiments, such proteins have at least 50% sequence identity, at least 60% sequence identity, at least 70% sequence identity, at least 80% sequence identity, at least 85% sequence identity, at least 95% sequence identity, at least 97% sequence identity, or at least 99% sequence identity.

As used herein, the term infectious agent refers to any microorganism capable of infecting a mammal. Preferred infectious agents are those which cause illness. Examples of infectious agents include, but are not limited to, viruses, bacteria and parasites. Examples of useful viruses for practicing methods of the present invention include, but are not limited to, viruses from a family selected from the group consisting of orthomyxoviridae, retroviridae, flaviviridae, filoviridae, coronoviridae, paramyxoviridae, picornoviridae, retroviridae, papillomaviridae, togaviridae, and polyomaviridae. More specific examples of useful viruses for practicing methods of the present invention include, but are not limited to, influenza viruses, human immunodeficiency viruses (HIV), flaviviruses (e.g., hepatitis virus, dengue virus, etc.), human papillomaviruses (HPV), rhinoviruses, coronaviruses, enteroviruses, polyomaviruses, respiratory synctial viruses (RSV), human metapneumoviruses, ebola viruses, Marburg viruses, alphaviruses (e.g., Chikungunya virus, Ross River virus, Semliki Forest virus, Sindbis virus, Mayaro virus, etc), Porcine Epidemic Diarrhea virus, Porcine reproductive and respiratory syndrome virus, and foot and mouth disease virus.

Proteins from infectious agents can be any protein useful for generating an immune response against an infectious agent comprising the protein. Useful proteins are those that elicit a protective immune response, such as the production of neutralizing antibodies. A particularly desirable protein is one that elicits the production of broadly neutralizing antibodies. One example of a useful protein with which to practice the present invention is the HIV envelope glycoprotein protein (Gp120). The ability of GP120 to elicit an antibody response, as well as useful mutants thereof, as well as other useful HIV proteins are described in U.S. Patent Publication Nos. US20140322269, US 20040052821, US20030064361, US20030158134, all of which are incorporated herein by reference in their entirety. Another example of a useful protein with which to practice the present invention is the flavivirus envelope protein, which is described in U.S. Patent Publication No. 20110059131, U.S. Patent Publication No. 20090311287, and U.S. Patent Publication No. 20040009469, all of which are incorporated herein by reference in their entirety. Another example of a useful protein with which to practice the present invention is the HCV capsid protein, which is described in U.S. Patent Publication No. 20020107360, U.S. Patent Publication No. 20020119495, and U.S. Patent Publication No. 20050233316, all of which are incorporated herein by reference in their entirety. Other useful proteins with which to practice the present invention are human Papillomavirus (HPV) proteins such as E2. The use of such proteins is described in U.S. Patent Publication No. 20100143408 and U.S. Patent Publication No. 20100183648, both of which are incorporated herein by reference in their entirety. Other useful proteins are disclosed in U.S. Patent Publication No. 20140161833, U.S. Patent Publication No. 20090202583, U.S. Patent Publication No. 20060182762, U.S. Patent Publication No. 20050053622, U.S. Patent Publication No. 20040175395, U.S. Patent Publication No. 20090162395, U.S. Patent Publication No. 20030224015, U.S. Patent Publication No. 20050255123, U.S. Patent Publication No. US 2012-0003266 and U.S. Patent Publication No. 20120315270, all of which are incorporated herein by reference in their entirety.

As used herein, broadly neutralizing antibodies are antibodies that neutralize an infectious agent from a taxonomic group that differs from the taxonomic groups of the infectious agents from which the immunogenic portions used to elicit the antibodies (used produce the nanoparticles) were derived. In preferred embodiments, nanoparticles of the invention elicit broadly neutralizing antibodies that neutralize at least one infectious agent from a genera, type, subtype, species and/or strain that differs from the genera, type, subtype, species and/or strain of the infectious agents from which immunogenic portions were derived in order to produce the nanoparticle. For example, if a nanoparticle is constructed using immunogenic portions of HA proteins from influenza A/Hong Kong/1/1968 (H3N2) and influenza A/Indonesia/05/2005 (H5N1), antibodies elicited by such nanoparticle and that that are broadly neutralizing would be able to neutralize one or more influenza viruses of genera, types, subtypes, species and/or strains other than influenza A/Hong Kong/1/1968 (H3N2) and influenza A/Indonesia/05/2005 (H5N1).

One embodiment of the present invention is a nanoparticle comprising fusion proteins, wherein the surface of the nanoparticle displays immunogenic portions of corresponding proteins from at least two infectious agents, wherein the at least two infectious agents are from different corresponding taxonomic groups within the same taxonomic family. In on embodiment, each fusion protein comprises at least a portion of a self-assembling, monomeric subunit joined to at least one portion of an immunogenic portion of a protein from an infectious agent. In one embodiment, the portion of the self-assembling, monomeric subunit comprises at least 25 amino acids, at least 50 amino acids, at least 75 amino acids, at least 100 amino acids or at least 150 amino acids from a monomeric subunit protein selected from the group consisting of a monomeric ferritin subunit protein, a monomeric encapsulin protein, a monomeric 03-33 protein, a monomeric SOR protein, a monomeric LS protein, a monomeric PDC protein and a Chikungunya virus structural polyprotein. In one embodiment, each fusion protein comprises a monomeric subunit protein selected from the group consisting of a monomeric ferritin subunit protein, a monomeric encapsulin protein, a monomeric 03-33 protein, a monomeric SOR protein, a monomeric LS protein, a monomeric PDC protein and a Chikungunya virus structural polyprotein.

In one embodiment, the infectious agents are viruses. Any virus capable of infecting a mammal can be used in constructing nanoparticles of the present invention. Examples of useful viruses for practicing methods of the present invention include, but are not limited to, viruses from a family selected from the group consisting of orthomyxoviridae, retroviridae, flaviviridae, filoviridae, coronoviridae, paramyxoviridae, picornoviridae, retroviridae, papillomaviridae, togaviridae, and polyomaviridae. Examples of useful viruses include, but are not limited to, influenza viruses, human immunodeficiency viruses (HIV), flaviviruses (e.g., hepatitis virus, dengue virus, etc.), human papillomaviruses (HPV), rhinoviruses, coronaviruses, enteroviruses, polyomaviruses, respiratory synctial viruses (RSV), human metapneumoviruses, ebola viruses, Marburg viruses, alphaviruses (e.g., Chikungunya virus, Ross River virus, Semliki Forest virus, Sindbis virus, Mayaro virus, etc), Porcine Epidemic Diarrhea virus, Porcine reproductive and respiratory syndrome virus and foot and mouth disease virus.

In one embodiment, the at least two infectious agents are from different genera within the same family. In one embodiment, the at least two infectious agents are from different species within the same family. In one embodiment, the at least two infectious agents are from different Types within the same family. In one embodiment, the at least two infectious agents are from different subtypes within the same family. In one embodiment, the at least two infectious agents are different strains within the same family.

One embodiment of the present invention is a nanoparticle comprising a first fusion protein and a second fusion protein, each fusion protein comprising at least a portion of a self-assembling, monomeric subunit joined to at least one immunogenic portion of a protein from an infectious agent, wherein the immunogenic portion of the first fusion protein is from a protein from a first infectious agent; wherein the immunogenic portion of the second fusion protein is from a protein from a second infectious agent; wherein the proteins from the first and second infectious agents are corresponding proteins; and wherein the first and second infectious agents are from different corresponding taxonomic groups within the same taxonomic family.

In one embodiment, the infectious agents are viruses. Any virus capable of infecting a mammal can be used in constructing nanoparticles of the present invention. Examples of useful viruses for practicing methods of the present invention include, but are not limited to, viruses from a family selected from the group consisting of orthomyxoviridae, retroviridae, flaviviridae, filoviridae, coronoviridae, paramyxoviridae, picornoviridae, retroviridae, papillomaviridae, togaviridae, and polyomaviridae. Examples of useful viruses include, but are not limited to, influenza viruses, human immunodeficiency viruses (HIV), flaviviruses (e.g., hepatitis virus, dengue virus, etc.), human papillomaviruses (HPV), rhinoviruses, coronaviruses, enteroviruses, polyomaviruses, respiratory synctial viruses (RSV), human metapneumoviruses, ebola viruses, Marburg viruses, alphaviruses (e.g., Chikungunya virus, Ross River virus, Semliki Forest virus, Sindbis virus, Mayaro virus, etc), Porcine Epidemic Diarrhea, Porcine reproductive and respiratory syndrome virus and foot and mouth disease virus.

In one embodiment, the at least two infectious agents are from different genera within the same family. In one embodiment, the at least two infectious agents are from different species within the same family. In one embodiment, the at least two infectious agents are from different Types within the same family. In one embodiment, the at least two infectious agents are from different subtypes within the same family. In one embodiment, the at least two infectious agents are different strains within the same family.

One embodiment of the present invention is a nanoparticle comprising at least two species of fusion protein, each species of fusion protein comprising at least a portion of a self-assembling, monomeric subunit joined to at least one immunogenic portion of a protein from a unique infectious agent, wherein the proteins in the unique infectious agents correspond to one another, and; wherein each unique infectious agent is from a different corresponding taxonomic group within the same taxonomic family.

As used herein, a unique infectious agent refers to infectious agents from the same taxonomic family, such as orthomyoviridae or retroviridae, which are genetically distinct from one another. Thus, infectious agents that are unique from one another would belong to different taxonomic groups. For example, two different strains of influenza virus would be considered unique from one another. Likewise, two different subtypes of influenza virus would be considered unique from one another.

While not intending to be limited to a specific embodiment, the inventors have chosen to utilize influenza virus to demonstrate the general principles and concepts of the present invention. Thus, with regard to certain embodiments of the present invention, all nomenclature used herein to classify influenza virus is that commonly used by those skilled in the art. Thus, a Type of influenza virus refers to influenza Type A, influenza Type B or influenza type C. It is understood by those skilled in the art that the designation of a virus as a specific Type relates to sequence difference in the respective M1 (matrix) protein or NP (nucleoprotein). Type A influenza viruses are further divided into Group1 and Group 2. These Groups are further divided into subtypes, a designation that refers to classification of a virus based on the sequence of its HA protein. Examples of current commonly recognized subtypes are H1, H2, H3, H4, H5, H6, H7, H8, H9, H10, H11, H12, H13, H14, H15, H16, H17 or H18. Group 1 influenza subtypes are H1, H2, H5, H6, H8, H9, H11, H12, H13, H16, H17 and H18. Group 2 influenza subtypes are H3, H4, H7, H10, H14 and H15. Finally, the term strain refers to viruses within a subtype that differ from one another due to small, genetic variations in their genome. Such genetic variations may, or may not, result in amino acid changes in the encoded influenza protein(s).

As used herein, an influenza hemagglutinin protein, or HA protein, refers the hemagglutinin glycoprotein present on the surface of influenza virus. Influenza virus HA proteins are able to bind sialic acid on the surface of cells, an activity responsible for the viruses ability to cause red blood cells to agglutinate. Influenza virus HA proteins are also responsible for fusion of the influenza virus membrane with the endosome membrane following infection of a cell by influenza virus. Such proteins, and their activities, are known to those skilled in the art. With specific regard to the present invention, an HA protein refers to a full-length influenza virus hemagglutinin protein or any portion thereof, that is, at least, capable of eliciting an immune response. Exemplary influenza proteins useful for producing nanoparticles of the present invention are listed below in Table 1.

TABLE 1

| SEQ ID | | |
|---|---|---|
| NOs. | ORGANISM | DESCRIPTION |
| | | Ectodomains |
| 1 | Influenza Virus | Amino acid sequence of ectodomain from the hemagglutinin |
| | | protein from A/New Caledonia/20/1999 (H1N1). |
| 2 | Influenza Virus | Amino acid sequence of ectodomain from the hemagglutinin |
| | | protein from A/California/04/2009 (H1N1) |
| 3 | Influenza Virus | Amino acid sequence of ectodomain from the hemagglutinin |
| | | protein from A/Singapore/1/1957 (H2N2) |
| 4 | Influenza Virus | Amino acid sequence of ectodomain from the hemagglutinin |
| | | protein from A/Hong Kong/1/1968 (H3N2) |
| 5 | Influenza Virus | Amino acid sequence of ectodomain from the hemagglutinin |
| | | protein from A/Brisbane/10/2007 (H3N2) |
| 6 | Influenza Virus | Amino acid sequence of ectodomain from the hemagglutinin |
| | | protein from A/Indonesia/05/2005 (H5N1) |
| 7 | Influenza Virus | Amino acid sequence of ectodomain from the hemagglutinin |
| | | protein from B/Florida/4/2006 (influenza B) |
| 8 | Influenza Virus | Amino acid sequence of ectodomain from the hemagglutinin |
| | | protein from A/Perth/16/2009 (H3N2) |
| 9 | Influenza Virus | Amino acid sequence of ectodomain from the hemagglutinin |
| | | protein from A/Brisbane/59/2007 (H1N1) |
| 10 | Influenza Virus | Amino acid sequence of ectodomain from the hemagglutinin |
| | | protein from B/Brisbane/60/2008 (influenza B) |
| 11 | Influenza Virus | Amino acid sequence of ectodomain from the hemagglutinin |
| | | protein from A/Wilson-Smith/33 (H1N1) |
| 12 | Influenza Virus | Amino acid sequence of ectodomain from the hemagglutinin |
| | | protein from A/Tientsin/78/77 (H1N1) |
| 13 | Influenza Virus | Amino acid sequence of ectodomain from the hemagglutinin |
| | | protein from A/Texas/36/91 (H1N1) |
| 14 | Influenza Virus | Amino acid sequence of ectodomain from the hemagglutinin |
| | | protein from A/Singapore/6/86 (H1N1) |
| 15 | Influenza Virus | Amino acid sequence of ectodomain from the hemagglutinin |
| | | protein from A/Memphis/39/83 (H1N1) |
| 16 | Influenza Virus | Amino acid sequence of ectodomain from the hemagglutinin |
| | | protein from A/Malaysia/54 (H1N1) |
| 17 | Influenza Virus | Amino acid sequence of ectodomain from the hemagglutinin |
| | | protein from A/Iowa/43 (H1N1) |
| 18 | Influenza Virus | Amino acid sequence of ectodomain from the hemagglutinin |
| | | protein from A/Hong Kong/117/77 (H1N1) |
| 19 | Influenza virus | Amino acid sequence of ectodomain from the hemagglutinin |
| | | protein from A/Fort Monmouth/1/47 (H1N1) |
| 20 | Influenza virus | Amino acid sequence of ectodomain from the hemagglutinin |
| | | protein from A/Brisbane/59/07 (H1N1) |
| 21 | Influenza Virus | Amino acid sequence of ectodomain from the hemagglutinin |
| | | protein from A/Baylor/4052/81 (H1N1) |
| 22 | Influenza Virus | Amino acid sequence of ectodomain from the hemagglutinin |
| | | protein from A/Albany/4835/48 (H1N1) |
| 23 | Influenza Virus | Amino acid sequence of ectodomain from the hemagglutinin |
| | | protein from A/Hong Kong/156/97 (H5N1) |
| 24 | Influenza Virus | Amino acid sequence of ectodomain from the hemagglutinin |
| | | protein from A/common magpie/Hong Kong/5052/07 (H5N1) |
| 25 | Influenza Virus | Amino acid sequence of ectodomain from the hemagglutinin |
| | | protein from A/chicken/Shanxi/2/06 (H5N1) |
| 26 | Influenza Virus | Amino acid sequence of ectodomain from the hemagglutinin |
| | | protein from A/silky chicken/Hong Kong/SF189/01 (H5N1) |
| 27 | Influenza Virus | Amino acid sequence of ectodomain from the hemagglutinin |
| | | protein from A/chicken/Henan/16/04 (H5N1) |
| 28 | Influenza Virus | Amino acid sequence of ectodomain from the hemagglutinin |
| | | protein from A/Victoria/361/11 (H3N2) |
| 29 | Influenza Virus | Amino acid sequence of ectodomain from the hemagglutinin |
| | | protein from B/Massachusetts/2/12 (Influenza B) |
| 30 | Influenza Virus | Amino acid sequence of ectodomain from the hemagglutinin |
| | | protein from B/Brisbane/60/08 (Influenza B) |
| 31 | Influenza Virus | Amino acid sequence of ectodomain from the hemagglutinin |
| | | protein from A/Texas/50/12 (H3N2) |
| | | Receptor Binding Domains |
| 32 | Influenza Virus | Amino acid sequence of RBD from hemagglutinin protein of |
| | | A/New Caledonia/20/1999 (H1N1); (56-264, F264A) |
| 33 | Influenza Virus | Amino acid sequence of RBD from hemagglutinin protein of |
| | | A/New Caledonia/20/1999 (H1N1); (56-264, Y98F, F264A) |
| 34 | Influenza Virus | Amino acid sequence of RBD from hemagglutinin protein of |
| | | A/California/04/2009 (H1N1); (56-264) |
| 35 | Influenza Virus | Amino acid sequence of RBD from hemagglutinin protein of |
| | | A/California/04/2009 (H1N1); (56-264, Y98F) |
| 36 | Influenza Virus | Amino acid sequence of RBD from hemagglutinin protein of |
| | | A/Singapore/1/1957 (H2N2) |
| 37 | Influenza Virus | Amino acid sequence of RBD from hemagglutinin protein of |
| | | A/Hong Kong/1/1968 (H3N2) |
| 38 | Influenza Virus | Amino acid sequence of RBD from hemagglutinin protein of |
| | | A/Brisbane/10/2007 (H3N2) |
| 39 | Influenza Virus | Amino acid sequence of RBD from hemagglutinin protein of |
| | | B/Florida/4/2006 (influenza B) |
| 40 | Influenza Virus | Amino acid sequence of RBD from hemagglutinin protein of |
| | | A/Perth/16/2009 (H3N2) |
| 41 | Influenza Virus | Amino acid sequence of RBD from hemagglutinin protein of |
| | | A/Wilson-Smith/33 (H1N1) (56-264, Y98F, F264A) |
| 42 | Influenza Virus | Amino acid sequence of RBD from hemagglutinin protein of |
| | | A/Tientsin/78/77 (H1N1) (56-264, Y98F, F264A) |
| 43 | Influenza Virus | Amino acid sequence of RBD from hemagglutinin protein of |
| | | A/Texas/36/91 (H1N1) (56-264, Y98F, F264A) |
| 44 | Influenza Virus | Amino acid sequence of RBD from hemagglutinin protein of |
| | | A/Singapore/6/86 (H1N1) (56-264, Y98F, F264A) |
| 45 | Influenza Virus | Amino acid sequence of RBD from hemagglutinin protein of |
| | | A/Memphis/39/83 (H1N1) (56-264, Y98F, F264A) |
| 46 | Influenza Virus | Amino acid sequence of RBD from hemagglutinin protein of |
| | | A/Malaysia/54 (H1N1) (56-264, Y98F) (56-264, Y98F) |
| 47 | Influenza Virus | Amino acid sequence of RBD from hemagglutinin protein of |
| | | A/Iowa/43 (H1N1) (56-264, Y98F, F264A) |
| 48 | Influenza Virus | Amino acid sequence of RBD from hemagglutinin protein of |
| | | A/Hong Kong/117/77 (H1N1) (56-264, Y98F, F264A) |
| 49 | Influenza Virus | Amino acid sequence of RBD from hemagglutinin protein of |
| | | Fort Monmouth/1/47 (H1N1) (56-264, Y98F, F264A) |
| 50 | Influenza virus | Amino acid sequence of RBD from hemagglutinin protein of |
| | | Brisbane/59/07 (H1N1) (56-264, Y98F, F264A) |
| 51 | Influenza Virus | Amino acid sequence of RBD from hemagglutinin protein of |
| | | A/Baylor/4052/81 (H1N1) (56-264, Y98F, F264A) |
| 52 | Influenza Virus | Amino acid sequence of RBD from hemagglutinin protein of |
| | | A/Albany/4835/48 (H1N1) (56-264, Y98F, F264A) |
| 53 | Influenza virus | Amino acid sequence of RBD from hemagglutinin protein of |
| | | Indonesia/05/05 (H5N1) (56-264, Y98F) |
| 54 | Influenza Virus | Amino acid sequence of RBD from hemagglutinin protein of |
| | | A/Hong Kong/156/97 (H5N1) (56-264, Y98F) |
| 55 | Influenza Virus | Amino acid sequence of RBD from hemagglutinin protein of |
| | | A/common magpie/Hong Kong/5052/07 (H5N1) (56-264, Y98F) |
| 56 | Influenza Virus | Amino acid sequence of RBD from hemagglutinin protein of |
| | | A/chicken/Shanxi/2/06 (H5N1) (56-264, Y98F) |
| 57 | Influenza Virus | Amino acid sequence of RBD from hemagglutinin protein of |
| | | A/silky chicken/Hong Kong/SF189/01 (H5N1) (56-264, Y98F) |
| 58 | Influenza Virus | Amino acid sequence of RBD from hemagglutinin protein of |
| | | A/chicken/Henan/16/04 (H5N1) (56-264, Y98F) |
| 59 | Influenza Virus | Amino acid sequence of RBD from hemagglutinin protein of |
| | | A/Victoria/361/11 (H3N2) (56-264, Y98F, K264A)) |
| 60 | Influenza Virus | Amino acid sequence of RBD from hemagglutinin protein of |
| | | B/Massachusetts/2/12 (Influenza B) (63-294) |
| 61 | Influenza Virus | Amino acid sequence of RBD from hemagglutinin protein of |
| | | B/Brisbane/60/08 (Influenza B) (63-294) |
| 62 | Influenza Virus | Amino acid sequence of RBD from hemagglutinin protein of |
| | | A/Texas/50/12 (H3N2) |
| | | Self-Assembly Proteins |
| | | Ferritin Proteins |
| 63 | Helicobacter pylori | Coding sequence for ferritin monomeric subunit protein from H. |
| | | pylori |
| 64 | Helicobacter pylori | Amino acid sequence encoded by SEQ ID NO: 63 |
| 65 | Helicobacter pylori | Complement of SEQ ID NO: 63 |
| 66 | Escherichia coli | Coding sequence for ferritin monomeric subunit protein from E. |
| | | coli (gi 446839951_WP_000917207.1) |
| 67 | Escherichia coli | Amino acid sequence encoded by SEQ ID NO: 66 |
| 68 | Escherichia coli | Complement of SEQ ID NO: 66 |
| 69 | Rana catesbeiana | Coding sequence for bullfrog ferritin monomeric subunit protein |
| | | (gi 13675 gb AAA49524.1) |
| 70 | Rana catesbeiana | Amino acid sequence encoded by SEQ ID NO: 69 |
| | | SEQ ID NO: 8 from 6137NIAID-34-PCT |
| 71 | Rana catesbeiana | Complement of SEQ ID NO: 69 |
| | | Hybrid Ferritin Proteins |
| 72 | Artificial Sequence | Coding sequence for H. pylori-ferritin/bullfrog-ferritin fusion |
| | | protein |
| 73 | Artificial Sequence | Amino acid sequence encoded by SEQ ID NO: 72 |
| 74 | Artificial Sequence | Complement of SEQ ID NO: 72 |
| 75 | Artificial Sequence | Coding sequence for E. coli-ferritin/bullfrog-ferritin fusion |
| | | protein |
| 76 | Artificial Sequence | Amino acid sequence encoded by SEQ ID NO: 75 |
| 77 | Artificial Sequence | Complement of SEQ ID NO: 75 |
| | | Other Self-Assembling Monomeric Subunits |
| 78 | Thermotoga maritima | Coding sequence for encapsulin protein |
| 79 | Thermotoga maritime | Amino acid sequence encoded by SEQ ID NO: 78 |
| 80 | Thermotoga maritime | Complement of SEQ ID NO: 78 |
| 81 | Artificial Sequence | Coding sequence for Salmonella enteritis 03-33 protein (gi |
| | | 390136278 pdb 3VCD) |
| 82 | Artificial Sequence | Amino acid sequence encoded by SEQ ID NO: 81 |
| 83 | Artificial Sequence | Complement of SEQ ID NO: 81 |
| 84 | Acidianus | Coding sequence for sulfur oxygenase reductase protein from |
| | ambivalens | Acidianus ambivalens (gi 93279016 pdb 2CB2) |
| 85 | Acidianus | Amino acid sequence encoded by SEQ ID NO: 84 |
| | ambivalens | |
| 86 | Acidianus | Complement of SEQ ID NO: 84 |
| | ambivalens | |
| 87 | Aquifex aeolicus | Coding sequence for lumazine synthase protein from Aquifex |
| | | aeolicus (gi 18159011 pdb1HQK) |
| 88 | Aquifex aeolicus | Amino acid sequence encoded by SEQ ID NO: 87 |
| 89 | Aquifex aeolicus | Complement of SEQ ID NO: 87 |
| 90 | Bacillus | Coding sequence for dihydrolipoamide acetyltransferase (E2p) |
| | stearothermophilus | protein from Bacillus stearothermophilus (gi 4558102 pdb1B5S |
| 91 | Bacillus | Amino acid sequence encoded by SEQ ID NO: 90 |
| | stearothermophilus | |
| 92 | Bacillus | Complement of SEQ ID NO: 90 |
| | stearothermophilus | |
| 93 | Chikungunya virus | Coding sequence for Chikungunya virus capsid, envelope E3, |
| | | E2, 6K, and E1 polyprotein |
| 94 | Chikungunya virus | Amino acid sequence encoded by SEQ ID NO: 93 |
| 95 | Chikungunya virus | Complement of SEQ ID NO: 93 |
| | | Ferritin Fusion Proteins |
| 96 | Artificial Sequence | Nucleic acid sequence of H1 NC99 RBD-Ferritin (56-264, |
| | | F264A) |
| 97 | Artificial Sequence | Amino acid sequence of H1 NC99 RBD-Ferritin (56-264, |
| | | F264A) |
| 98 | Artificial Sequence | Complement of SEQ ID NO: 96 |
| 99 | Artificial Sequence | Nucleic acid sequence of H1 NC99 RBD-Ferritin (56-264, Y98F, |
| | | F264A) |
| 100 | Artificial Sequence | Amino acid sequence of H1 NC99 RBD-Ferritin (56-264, |
| | | F264A) |
| 101 | Artificial Sequence | Complement of SEQ ID NO: 99 |
| 102 | Artificial Sequence | Nucleic acid sequence of H1 CA09 RBD-Ferritin (56-264) |
| 103 | Artificial Sequence | Amino acid sequence of H1 CA09 RBD-Ferritin (56-264) |
| 104 | Artificial Sequence | Complement of SEQ ID NO: 102 |
| 105 | Artificial Sequence | Nucleic acid sequence of H1 CA09 RBD-Ferritin (56-264, |
| | | Y98F) |
| 106 | Artificial Sequence | Amino acid sequence of H1 CA09 RBD-Ferritin (56-264, Y98F) |
| 107 | Artificial Sequence | Complement of SEQ ID NO: 105 |
| 108 | Artificial Sequence | Nucleic acid sequence of H1 WS33 RBD-Ferritin (56-264, |
| | | Y98F, F264A) |
| 109 | Artificial Sequence | Amino acid sequence of H1 WS33 RBD-Ferritin (56-264, Y98F, |
| | | F264A) |
| 110 | Artificial Sequence | Complement of SEQ ID NO: 108 |
| 111 | Artificial Sequence | Nucleic acid sequence of H1 Tien 77 RBD-Ferritin (56-264, |
| | | Y98F, F264A) |
| 112 | Artificial Sequence | Amino acid sequence of H1 Tien 77 RBD-Ferritin (56-264, |
| | | Y98F, F264A) |
| 113 | Artificial Sequence | Complement of SEQ ID NO: 111 |
| 114 | Artificial Sequence | Nucleic acid sequence of H1 TX91 RBD-Ferritin (56-264, Y98F, |
| | | F264A) |
| 115 | Artificial Sequence | Amino acid sequence of H1 TX91 RBD-Ferritin (56-264, Y98F, |
| | | F264A) |
| 116 | Artificial Sequence | Complement of SEQ ID NO: 114 |
| 117 | Artificial Sequence | Nucleic acid sequence of H1 SG86 RBD-Ferritin (56-264, Y98F, |
| | | F264A) |
| 118 | Artificial Sequence | Amino acid sequence of H1 SG86 RBD-Ferritin (56-264, Y98F, |
| | | F264A) |
| 119 | Artificial Sequence | Complement of SEQ ID NO: 117 |
| 120 | Artificial Sequence | Nucleic acid sequence of H1 Mem83 RBD-Ferritin (56-264, |
| | | Y98F, F264A) |
| 121 | Artificial Sequence | Amino acid sequence of H1 Mem83 RBD-Ferritin (56-264, |
| | | Y98F, F264A) |
| 122 | Artificial Sequence | Complement of SEQ ID NO: 120 |
| 123 | Artificial Sequence | Nucleic acid sequence of H1 Mal54 RBD-Ferritin (56-264, |
| | | Y98F) |
| 124 | Artificial Sequence | Amino acid sequence of H1 Mal54 RBD-Ferritin (56-264, Y98F) |
| 125 | Artificial Sequence | Complement of SEQ ID NO: 123 |
| 126 | Artificial Sequence | Nucleic acid sequence of H1 IA43 RBD-Ferritin (56-264, Y98F, |
| | | F264A) |
| 127 | Artificial Sequence | Amino acid sequence of H1 IA43 RBD-Ferritin (56-264, Y98F, |
| | | F264A) |
| 128 | Artificial Sequence | Complement of SEQ ID NO: 126 |
| 129 | Artificial Sequence | Nucleic acid sequence of H1 HK77 RBD-Ferritin (56-264, Y98F, |
| | | F264A) |
| 130 | Artificial Sequence | Amino acid sequence of H1 HK77 RBD-Ferritin (56-264, Y98F, |
| | | F264A) |
| 131 | Artificial Sequence | Complement of SEQ ID NO: 129 |
| 132 | Artificial Sequence | Nucleic acid sequence of H1 FM47 RBD-Ferritin (56-264, Y98F, |
| | | F264A) |
| 133 | Artificial Sequence | Amino acid sequence of H1 FM47 RBD-Ferritin (56-264, Y98F, |
| | | F264A) |
| 134 | Artificial Sequence | Complement of SEQ ID NO: 132 |
| 135 | Artificial Sequence | Nucleic acid sequence of H1 BRO7 RBD-Ferritin (56-264, |
| | | Y98F, F264A) |
| 136 | Artificial Sequence | Amino acid sequence of H1 BRO7 RBD-Ferritin (56-264, Y98F, |
| | | F264A) |
| 137 | Artificial Sequence | Complement of SEQ ID NO: 135 |
| 138 | Artificial Sequence | Nucleic acid sequence of H1 Bay81 RBD-Ferritin (56-264, |
| | | Y98F, F264A) |
| 139 | Artificial Sequence | Amino acid sequence of H1 Bay81 RBD-Ferritin (56-264, Y98F, |
| | | F264A) |
| 140 | Artificial Sequence | Complement of SEQ ID NO: 138 |
| 141 | Artificial Sequence | Nucleic acid sequence of H1 Alb48 RBD-Ferritin (56-264, |
| | | Y98F, F264A) |
| 142 | Artificial Sequence | Amino acid sequence of H1 Alb48 RBD-Ferritin (56-264, Y98F, |
| | | F264A) |
| 143 | Artificial Sequence | Complement of SEQ ID NO: 141 |
| 144 | Artificial Sequence | Nucleic acid sequence of H5 IN05 RBD-Ferritin (56-264, Y98F) |
| 145 | Artificial Sequence | Amino acid sequence of H5 IN05 RBD-Ferritin (56-264, Y98F) |
| 146 | Artificial Sequence | Complement of SEQ ID NO: 144 |
| 147 | Artificial Sequence | Nucleic acid sequence of H5 HK97(c0) RBD-Ferritin (56-264, |
| | | Y98F) |
| 148 | Artificial Sequence | Amino acid sequence of H5 HK97(c0) RBD-Ferritin (56-264, |
| | | Y98F) |
| 149 | Artificial Sequence | Complement of SEQ ID NO: 147 |
| 150 | Artificial Sequence | Nucleic acid sequence of H5 HK07(c2.3.2.1) RBD-Ferritin (56- |
| | | 264, Y98F) |
| 151 | Artificial Sequence | Amino acid sequence of H5 HK07(c2.3.2.1) RBD-Ferritin (56- |
| | | 264, Y98F) |
| 152 | Artificial Sequence | Complement of SEQ ID NO: 150 |
| 153 | Artificial Sequence | Nucleic acid sequence of H5 SX06(c7.2) RBD-Ferritin (56-264, |
| | | Y98F) |
| 154 | Artificial Sequence | Amino acid sequence of H5 SX06(c7.2) RBD-Ferritin (56-264, |
| | | Y98F) |
| 155 | Artificial Sequence | Complement of SEQ ID NO: 153 |
| 156 | Artificial Sequence | Nucleic acid sequence of H5 HK01(c3) RBD-Ferritin (56-264, |
| | | Y98F) |
| 157 | Artificial Sequence | Amino acid sequence of H5 HK01(c3) RBD-Ferritin (56-264, |
| | | Y98F) |
| 158 | Artificial Sequence | Complement of SEQ ID NO: 156 |
| 159 | Artificial Sequence | Nucleic acid sequence of H5 HN04(c8) RBD-Ferritin (56-264, |
| | | Y98F) |
| 160 | Artificial Sequence | Amino acid sequence of H5 HN04(c8) RBD-Ferritin (56-264, |
| | | Y98F) |
| 161 | Artificial Sequence | Complement of SEQ ID NO: 159 |
| 162 | Artificial Sequence | Nucleic acid sequence of H3 Vic11 RBD-Ferritin (56-264, Y98F, |
| | | K264A) |
| 163 | Artificial Sequence | Amino acid sequence of H3 Vic11 RBD-Ferritin (56-264, Y98F, |
| | | K264A) |
| 164 | Artificial Sequence | Complement of SEQ ID NO: 162 |
| 165 | Artificial Sequence | Nucleic acid sequence of B MA12 RBD-Ferritin (63-294) |
| 166 | Artificial Sequence | Amino acid sequence of B MA12 RBD-Ferritin (63-294) |
| 167 | Artificial Sequence | Complement of SEQ ID NO: 165 |
| 168 | Artificial Sequence | Nucleic acid sequence of B BRO8 RBD-Ferritin (63-295) |
| 169 | Artificial Sequence | Amino acid sequence of B BRO8 RBD-Ferritin (63-295) |
| 170 | Artificial Sequence | Complement of SEQ ID NO: 168 |
| | | Ferritin Single Polypeptide Design Fusion Proteins |
| 171 | Artificial Sequence | Nucleic acid sequence H1/H3 CA09 TX12 F2A RBD-Ferritin |
| 172 | Artificial Sequence | Amino acid sequence of H1/H3 CA09 TX12 F2A RBD-Ferritin |
| 173 | Artificial Sequence | Complement of SEQ ID NO: 171 |
| 174 | Artificial Sequence | Nucleic acid sequence of B/B BRO8 MA12 F2A RBD-Ferritin |
| 175 | Artificial Sequence | Amino acid sequence of B/B BRO8 MA12 F2A RBD-Ferritin |
| 176 | Artificial Sequence | Complement of SEQ ID NO: 174 |
| 177 | Artificial Sequence | Nucleic acid sequence H1/H3/B CA09 TX12 MA12 F2A RBD- |
| | | Ferritin |
| 178 | Artificial Sequence | Amino acid sequence of H1/H3 CA09 TX12 MA12 F2A RBD- |
| | | Ferritin |
| 179 | Artificial Sequence | Complement of SEQ ID NO: 177 |
| | | Encapsulin Fusion Proteins |
| 180 | Artificial Sequence | Nucleic acid sequence of H1 NC99 RBD-Encapsulin (56-264, |
| | | F264A) |
| 181 | Artificial Sequence | Amino acid sequence of H1 NC99 RBD-Encapsulin (56-264, |
| | | F264A) |
| 182 | Artificial Sequence | Complement of SEQ ID NO: 180 |
| 183 | Artificial Sequence | Nucleic acid sequence of H1 CA09 RBD-Encapsulin (56-264) |
| 184 | Artificial Sequence | Amino acid sequence of H1 CA09 RBD-Encapsulin (56-264) |
| 185 | Artificial Sequence | Complement of SEQ ID NO: 183 |
| | | CHIK VLP Fusion Proteins |
| 186 | Artificial Sequence | Nucleic acid sequence of H1 NC99 RBD-CHIKVLP (59-264, |
| | | Y98F, F264A) |
| 187 | Artificial Sequence | Amino acid sequence of NC99 RBD-CHIKVLP (59-264, Y98F, |
| | | F264A) |
| 188 | Artificial Sequence | Complement of SEQ ID NO: 186 |
| 189 | Artificial Sequence | Nucleic acid sequence of H1 CA09 RBD-CHIKVLP (59-264, |
| | | Y98F) |
| 190 | Artificial Sequence | Amino acid sequence of H1 CA09 RBD-CHIKVLP (59-264, |
| | | Y98F) |
| 191 | Artificial Sequence | Complement of SEQ ID NO: 189 |

It is understood by those skilled in the art that HA proteins from different influenza viruses may have different lengths, due to insertions and/or deletions of amino acid residue in one or both of the HA proteins. Thus, reference to a corresponding region refers to a region of another proteins that is identical, or nearly so (e.g., at least 95%, identical, at least 98% identical or at least 99% identical), in sequence, structure and/or function to the region being compared. For example, with regard to the globular head region or RBD of a hemagglutinin protein, the corresponding region in another hemagglutinin protein may not have the same residue numbers, but will have a very similar sequence and will perform the same function. To improve sequence comparisons between viruses, numbering systems are used by those in the field, which relate amino acid positions to a reference sequence. Thus, corresponding amino acid residues in hemagglutinin proteins from different strains of influenza may not have the same residue number with respect to their distance from the N-terminal amino acid of the mature protein. For example, using the H3 numbering system, reference to residue 100 in A/New Caledonia/20/1999 (1999 NC, H1) does not mean it is the 100th residue from the N-terminal amino acid of the mature protein. Instead, residue 100 of A/New Caledonia/20/1999 (1999 NC, H1) HA protein aligns with residue 100 of the HA protein from influenza H3N2 strain. The use of such numbering systems is understood by those skilled in the art. Unless otherwise noted, reference to amino acid positions in hemagglutinin proteins herein is made using the H3 numbering system.

As used herein, the term immunogenic refers to the ability of a specific protein, or a specific region thereof (i.e., a specific amino acid sequence), to elicit an immune response to the specific protein, or to proteins comprising an amino acid sequence having a high degree of identity with the specific protein. According to the present invention, two proteins having a high degree of identity have amino acid sequences at least 80% identical, at least 85% identical, at least 87% identical, at least 90% identical, at least 92% identical, at least 94% identical, at least 96% identical, at least 98% identical or at least 99% identical. Preferred immunogenic proteins, or portions thereof, are those that elicit neutralizing antibodies to influenza virus.

As used herein, a heterogeneous population refers to a population of molecules in which at least one molecule in the population differs in sequence from at least one other molecule in the population. For example, with particular regard to the present invention, in a heterogeneous population of immunogenic portions from influenza HA proteins, the population is heterologous due to the fact that the amino acid sequence of at least one immunogenic portion in the population differs from the amino acid sequence of at least one other immunogenic portion in the population. With regard to the present invention, each unique sequence is referred to as a species of molecule (e.g., a species of immunogenic portion, a species of fusion protein, etc.). The difference in sequence between two species of molecule can involve a single amino acid difference or it can involve more than one amino acid difference. Moreover, such differences may, or may not, result in different species having different epitopes.

As used herein, epitopes are clusters of amino acid residues that are recognized by (e.g., bound by) components of the immune system, such as B-cell receptors, T-cell receptors, antibodies, and the like, thus forming an immune complex and eliciting an immune response. Such epitopes may consist of contiguous amino acids residues (i.e., amino acid residues that are adjacent to one another in the protein), or they may consist of non-contiguous amino acid residues (i.e., amino acid residues that are not adjacent one another in the linear protein) but which are in close spatial proximity in three-dimensional space in the finally folded protein. Thus, in one embodiment the immunogenic portion comprises at least one epitope from an influenza virus HA protein.

As used herein, a monovalent nanoparticle refers to a nanoparticle that displays a single species of immunogenic portion from an HA protein on its surface. That is, all of the immunogenic portions have the same sequence. As used herein, Admixed nanoparticles refers to a population of nanoparticles that contains a mixture of monovalent nanoparticle species. In such a population, each monovalent nanoparticle is produced separately from other monovalent nanoparticles, and the monovalent nanoparticles are then mixed together to produce Admixed nanoparticles. It will be understood by those skilled in the art that while a population of Admixed nanoparticles comprises more than one species of immunogenic portion, each monovalent nanoparticle in the Admixed population comprises a single species of immunogenic portion. As used herein, a multivalent co-assembled nanoparticle, co-assembled nanoparticle, and the like, refers to a nanoparticle made by combining more than one species of fusion protein, wherein at least two fusion proteins differ in the sequence of their immunogenic portions. The result is a nanoparticle comprising a heterogeneous population of self-assembling fusion proteins, wherein the nanoparticle displays on its surface a heterogeneous population of immunogenic portions from HA proteins. Such multivalent nanoparticles can also be referred to as mosaic nanoparticles.

One embodiment of the present invention is a nanoparticle comprising a heterologous population of fusion proteins, wherein each fusion protein comprises at least one immunogenic portion from an influenza HA protein joined to at least 25 contiguous amino acids from a monomeric subunit protein capable of assembling into a nanoparticle (i.e., a self-assembly (SA) protein), wherein the heterologous population of fusion proteins comprises at least two different species of fusion proteins. Nanoparticles of the present invention can be made from fusion proteins comprising immunogenic portions of HA proteins from any Type, sub-type, strain, or combinations thereof, of influenza virus. In certain embodiments, the immunogenic portions are from HA proteins from one or more influenza viruses selected from the group consisting of Type A influenza viruses, Type B influenza viruses and Type C influenza viruses. In one embodiment, the immunogenic portions are from HA proteins from one or more virus selected from the group consisting of Group I influenza viruses and Group II influenza virus. In one embodiment, the immunogenic portions are from HA proteins from one or more virus selected from the group consisting of an H1 influenza virus, an H2 influenza virus, an influenza H3 virus, an influenza H4 virus, an influenza H5 virus, an influenza H6 virus, an H7 influenza virus, an H8 influenza virus, an H9 influenza virus, an H10 influenza virus, an H11 influenza virus, an H12 influenza virus, an H13 influenza virus, an H14 influenza virus, an H15 influenza virus, an H16 influenza virus, an H17 influenza virus, an H18 influenza virus and an influenza B lineage virus. In certain embodiments, the immunogenic portions are from HA proteins from one or more influenza viruses selected from the group consisting of A/New Caledonia/20/1999 (HIN1), A/California/04/2009 (H1N1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 (H1N1), A/Malaysia/54 (H1N1), A/Iowa/43 (H1N1), A/Hong Kong/117/77 (H1N1), A/Fort Monmouth/1/47 (H1N1), A/Brisbane/59/07 (H1N1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2). In certain embodiments, the immunogenic portions are from one or more HA proteins Listed in Table 1. In certain embodiments, the immunogenic portions are from one or more HA proteins selected from the group consisting of HA proteins comprising SEQ ID NO:1-SEQ ID NO:62. In certain embodiments, the immunogenic portions are from one or more HA proteins selected from the group consisting of HA proteins consisting of SEQ ID NO:1-SEQ ID NO:62.

Immunogenic portions useful for constructing nanoparticles of the present invention can also be obtained from variants of influenza virus HA proteins disclosed herein. As used herein, a variant refers to a protein, or nucleic acid molecule, the sequence of which is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as such alterations do not significantly affect the activity of the variant protein and the variant protein retains the desired activity. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein.

As noted, variant proteins of the present invention can contain amino acid substitutions relative to the influenza HA proteins disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

Naturally occurring residues may be divided into classes based on common side chain properties:
1) hydrophobic: Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

For example, non-conservative substitutions may involve the exchange of a member of one of these classes for a member from another class.

In making amino acid changes, the hydropathic index of amino acids may be considered. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is generally understood in the art (Kyte et al., 1982, J. Mol. Biol. 157:105-31). It is known that certain amino acids may be substituted for other amino acids having a similar hydropathic index or score and still retain a similar biological activity. In making changes based upon the hydropathic index, the substitution of amino acids whose hydropathic indices are within +2 is preferred, those within +1 are particularly preferred, and those within +0.5 are even more particularly preferred.

It is also understood in the art that the substitution of like amino acids can be made effectively on the basis of hydrophilicity, particularly where the biologically functionally equivalent protein or peptide thereby created is intended for use in immunological invention, as in the present case. The greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within +2 is preferred, those within +1 are particularly preferred, and those within +0.5 are even more particularly preferred. One may also identify epitopes from primary amino acid sequences on the basis of hydrophilicity.

Desired amino acid substitutions (whether conservative or non-conservative) can be determined by those skilled in the art at the time such substitutions are desired. For example, amino acid substitutions can be used to identify important residues of the HA protein, or to increase or decrease the immunogenicity, solubility or stability of the HA proteins described herein. Exemplary amino acid substitutions are shown below in Table 2.

TABLE 2
Amino Acid Substitutions

| Original Amino Acid | Exemplary Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala |
| Leu | Ile, Val, Met, Ala |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

As used herein, the phrase significantly affect a proteins activity refers to a decrease in the activity of a protein by at least 10%, at least 20%, at least 30%, at least 40% or at least 50%. With regard to the present invention, such activity may be ability to elicit antibodies, including neutralizing antibodies, against an influenza virus. The determination of antibody production may be measured by measuring the titer of such antibodies against influenza virus, or by measuring the number of types, subtypes or strains bound by the elicited antibodies. Methods of determining antibody titers and methods of performing virus neutralization assays are known to those skilled in the art. In addition to the activities described above, examples of other activities that may be measured include the ability to agglutinate red blood cells, the ability to bind sialic acid or the binding affinity of the protein for a cell. Methods of measuring such activities are known to those skilled in the art.

Thus, in one embodiment, nanoparticles of the present invention comprise fusion proteins comprising immunogenic portions from HA proteins comprising amino acid sequences at least 80%, at least 85%, at least 90%, at least 92%, at least 94%, at least 96%, at least 98% or at least 99% identical to amino acid sequences from one or more HA proteins from any Type, sub-type, strain, or combinations thereof, of influenza virus. In certain embodiments, the immunogenic portions are from HA proteins from one or more influenza viruses selected from the group consisting of Type A influenza virus, Type B influenza virus and Type C influenza viruses. In certain embodiments, the immunogenic portions are from HA proteins from one or more influenza viruses selected from the group consisting of Group 1 influenza virus and Group 2 influenza viruses. In one embodiment, the immunogenic portions are from one or more HA proteins comprising amino acid sequences at least 80%, at least 85%, at least 90%, at least 92%, at least 94%, at least 96%, at least 98% or at least 99% identical to amino acid sequences of HA proteins from one or more viruses selected from the group consisting of an H1 influenza virus, an H2 influenza virus, an influenza H3 virus, an influenza H4 virus, an influenza H5 virus, an influenza H6 virus, an H7 influenza virus, an H8 influenza virus, an H9 influenza virus, an H10 influenza virus, an H11 influenza virus, an H12 influenza virus, an H13 influenza virus, an H14 influenza virus, an H15 influenza virus, an H16 influenza virus, an H17 influenza virus, an H18 influenza virus and an influenza lineage B virus. In certain embodiments, the immunogenic portions are from HA proteins comprising amino acid sequence at least 80%, at least 85%, at least 90%, at least 92%, at least 94%, at least 96%, at least 98% or at least 99% identical to amino acid sequences of HA proteins from one or more influenza viruses selected from the group consisting of A/New Caledonia/20/1999 (H1N1), A/California/04/2009 (H1N1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 Monmouth/1/47 (H1N1), A/Brisbane/59/07 (H1N1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2). In certain embodiments, the immunogenic portions are from HA proteins comprising amino acid sequences at least 80%, at least 85%, at least 90%, at least 92%, at least 94%, at least 96%, at least 98% or at least 99% identical to amino acid sequences of one or more HA proteins Listed in Table 1. In certain embodiments, the immunogenic portions are from HA proteins comprising amino acid sequences at least 80%, at least 85%, at least 90%, at least 92%, at least 94%, at least 96%, at least 98% or at least 99% identical to amino acid sequences of one or more HA proteins selected from the group consisting of HA proteins comprising SEQ ID NO:1-SEQ ID NO:62. In certain embodiments, the immunogenic portions are from HA proteins comprising amino acid sequences at least 80%, at least 85%, at least 90%, at least 92%, at least 94%, at least 96%, at least 98% or at least 99% identical to amino acid sequences of one or more HA proteins selected from the group consisting of HA proteins consisting of SEQ ID NO:1-SEQ ID NO:62.

It is understood by those skilled in the art that the influenza HA protein contains different regions or domains. Examples of such regions include the stem region and the globular head region. Thus, while nanoparticle-based influenza vaccines can be made using immunogenic portions from any influenza HA proteins, in preferred embodiments the immunogenic portions are from a specific region or domain of the selected HA proteins. One embodiment of the present invention is a nanoparticle comprising a heterologous population of fusion proteins, wherein each fusion protein comprises at least one immunogenic portion from the globular head region of an influenza HA protein joined to at least 25 contiguous amino acids from a monomeric subunit protein capable of assembling into a nanoparticle (i.e., a self-assembly (SA) protein), wherein the heterologous population of fusion proteins comprises at least two different species of fusion proteins. The globular head region, which comprises (approximately) amino acid residues 52-277 of influenza A HA protein (H3 numbering system), consists exclusively of the major portion of the HA1 polypeptide and includes two domains: the receptor binding domain (RBD and the vestigial esterase sub-domain. One example of a globular head region is represented by amino acids 52-277 from an HA protein comprising a region corresponding to an amino acid sequence selected from the group consisting of SEQ ID NO:1-62. In one embodiment, the immunogenic portions are from the globular head regions of HA proteins comprising amino acid sequences at least 80%, at least 85%, at least 90%, at least 92%, at least 94%, at least 96%, at least 98% at least 99%, or which are, identical to the amino acid sequences of HA proteins from one or more influenza viruses selected from the group consisting of Type A influenza viruses, Type B influenza viruses and Type C influenza viruses. In one embodiment, the immunogenic portions are from the globular head regions of HA proteins comprising amino acid sequences at least 80%, at least 85%, at least 90%, at least 92%, at least 94%, at least 96%, at least 98% at least 99%, or which are, identical to the amino acid sequences of HA proteins from one or more influenza viruses selected from the group consisting of Group I influenza viruses and Group II influenza viruses. In one embodiment, the immunogenic portions are from globular head regions of HA proteins comprising amino acid sequences at least 80%, at least 85%, at least 90%, at least 92%, at least 94%, at least 96%, at least 98%, at least 99%, or which are, identical to the amino acid sequences of HA proteins from one or more viruses selected from the group consisting of an H1 influenza virus, an H2 influenza virus, an influenza H3 virus, an influenza H4 virus, an influenza H5 virus, an influenza H6 virus, an H7 influenza virus, an H8 influenza virus, an H9 influenza virus, an H10 influenza virus, an H11 influenza virus, an H12 influenza virus, an H13 influenza virus, an H14 influenza virus, an H15 influenza virus, an H16 influenza virus, a H17 influenza virus, an H18 influenza virus and an influenza linage B virus. In one embodiment, the immunogenic portions are from the globular head regions of HA proteins comprising amino acid sequences at least 80%, at least 85%, at least 90%, at least 92%, at least 94%, at least 96%, at least 98% at least 99%, or which are, identical to the amino acid sequences of HA proteins from one or more influenza viruses selected from the group consisting of A/New Caledonia/20/1999 (H1N1), A/California/04/2009 (H1N1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 (H1N1), A/Malaysia/54 (H1N1), A/Iowa/43 (H1N1), A/Hong Kong/117/77 (H1N1), A/Fort Monmouth/1/47 (H1N1), A/Brisbane/59/07 (H1N1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2). In one embodiment, the immunogenic portions are from the globular head regions of HA proteins comprising amino acid sequences at least 80%, at least 85%, at least 90%, at least 92%, at least 94%, at least 96%, at least 98% at least 99%, or which are, identical to the amino acid sequences of one or more HA proteins Listed in Table 1.

As has been discussed, the globular head region comprises several other regions or domains. Thus, it will be appreciated by those skilled in the art that the immunogenic portions of the self-assembling fusion proteins can be fragments from the globular head regions from one or more influenza virus HA proteins. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from globular head regions of influenza virus HA proteins. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the globular head regions of HA proteins, or variant, thereof, from one or more influenza viruses selected form the group consisting of Type A influenza viruses, Type B influenza viruses and Type C influenza viruses. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the globular head regions of HA proteins, or variants, thereof, from one or more influenza viruses selected from the group consisting of Group I influenza viruses and Group II influenza viruses. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the globular head regions of HA proteins, or variant thereof, from one or more viruses selected from the group consisting of an H1 influenza virus, an H2 influenza virus, an influenza H3 virus, an influenza H4 virus, an influenza H5 virus, an influenza H6 virus, an H7 influenza virus, an H8 influenza virus, an H9 influenza virus, an H10 influenza virus, an H11 influenza virus, an H12 influenza virus, an H13 influenza virus, an H14 influenza virus, an H15 influenza virus, an H16 influenza virus, an H17 influenza virus, an H18 influenza virus and an influenza lineage B virus. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the region corresponding to the globular head regions of HA proteins, or variant thereof, from one or more influenza viruses selected from the group consisting of A/New Caledonia/20/1999 (H1N1), A/California/04/2009 (HIN1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 (H1N1), A/Malaysia/54 (H1N1), A/Iowa/43 (H1N1), A/Hong Kong/117/77 (H1N1), A/Fort Monmouth/1/47 (H1N1), A/Brisbane/59/07 (H1N1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2). In one embodiment, the immunogenic portions comprises at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the regions corresponding to the globular head regions of one or more HA proteins, or variant thereof, listed in Table 1. In one embodiment, the immunogenic portions comprises at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from regions corresponding to the globular head regions of one or more HA proteins, or variant thereof, comprising sequences selected from the group consisting of SEQ ID NO:1-SEQ ID NO:31.

A particularly useful portion of the globular head region is the receptor-binding domain (RBD). The receptor-binding domain comprises (approximately) amino acid residues 56-264 of influenza A HA protein (H3 numbering system). One embodiment of the present invention is a nanoparticle comprising a heterologous population of fusion proteins, wherein each fusion protein comprises at least one immunogenic portion from the RBD of an influenza HA protein joined to at least 25 contiguous amino acids from a monomeric subunit protein capable of assembling into a nanoparticle (i.e., a self-assembly (SA) protein), wherein the heterologous population of fusion proteins comprises at least two different species of fusion proteins. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the receptor-binding domains (RBDs) of one or more influenza virus HA proteins. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the RBDs of HA proteins, or variants thereof, from one or more influenza viruses selected from the group consisting of Type A influenza viruses, Type B influenza viruses and Type C influenza viruses. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the RBDs of HA proteins, or variants thereof, from one or more influenza viruses selected from the group consisting of Group 1 influenza viruses and Group 2 influenza viruses. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the RBDs of HA proteins, or variants thereof, from one or more viruses selected from the group consisting of an H1 influenza virus, an H2 influenza virus, an influenza H3 virus, an influenza H4 virus, an influenza H5 virus, an influenza H6 virus, an H7 influenza virus, an H8 influenza virus, an H9 influenza virus, an H10 influenza virus, an H11 influenza virus, an H12 influenza virus, an H13 influenza virus, an H14 influenza virus, an H15 influenza virus, an H16 influenza virus, an H17 influenza virus, an H18 influenza virus and an influenza lineage B virus. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the RBDs of HA proteins, or variants thereof, from one or more influenza viruses selected from the group consisting of A/New Caledonia/20/1999 (H1N1), A/California/04/2009 (H1N1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 Monmouth/1/47 (H1N1), A/Brisbane/59/07 (H1N1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2). In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from the RBDs of one or more HA proteins, or variants thereof, Listed in Table 1. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from an amino acid sequence at least 80%, at least 85%, at least 90%, at least 92%, at least 94%, at least 96%, at least 98% or at least 99% identical to a sequence selected from the group consisting of SEQ ID NO:32-SEQ ID NO:62. In one embodiment, the immunogenic portions comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or at least 200 contiguous amino acid residues from one or more amino acid sequences selected from the group consisting of SEQ ID NO:32-SEQ ID NO:62. In one embodiment, the immunogenic portions comprise one or more amino acid sequence selected from the group consisting of SEQ ID NO:32-SEQ ID NO:62.

As described herein, in order to form nanoparticles expressing immunogenic portions of influenza HA proteins on theirs surfaces, each immunogenic portion is joined to a self-assembly (SA) subunit protein, or a functional portion or variant thereof, thereby forming a hemagglutinin-self-assembly (HA-SA) fusion protein. Upon expression, the HA-SA fusion proteins assemble into a nanoparticle that displays the immunogenic portion of the HA protein on its surface. Any self-assembly subunit protein, or variant thereof, can be used to produce a fusion protein of the present invention, as long as the resulting fusion protein is capable of self-assembling into a nanoparticle. Examples of self-assembly subunit proteins useful for constructing fusion proteins of the present invention include, but are not limited to, ferritin, encapsulin, sulfur oxygenase reductase, lumazine synthase, dihydrolipoamide acetyltransferase (E2), Chikungunya virus envelope proteins, and fragments and/or variants thereof.

In one embodiment, the self-assembly protein is ferritin. Ferritin, which is found in all animals, bacteria and plants, forms a spherical protein complex that acts primarily to control the rate and location of polynuclear Fe(III)₂O₃ formation through the transportation of hydrated iron ions and protons to and from a mineralized core. The spherical form of ferritin is made up of monomeric subunit proteins (also referred to as monomeric ferritin subunits), which are polypeptides having a molecule weight of approximately 17-20 kDa. Examples of the sequences of monomeric ferritin subunits are represented by SEQ ID NO:64, SEQ ID NO:67 and SEQ ID NO:69. Each monomeric ferritin subunit has the topology of a helix bundle which includes a four antiparallel helix motif, with a fifth shorter helix (the c-terminal helix) lying roughly perpendicular to the long axis of the 4 helix bundle. According to convention, the helices are labeled ‘A, B, C, and D & E’ from the N-terminus respectively. The N-terminal sequence lies adjacent to the capsid three-fold axis and extends to the surface, while the E helices pack together at the four-fold axis with the C-terminus extending into the particle core. The consequence of this packing creates two pores on the capsid surface. While not intended to be bound by theory, it is expected that one or both of these pores represent the point by which the hydrated iron diffuses into and out of the capsid. Following production, these monomeric ferritin subunit proteins self-assemble into the spherical ferritin protein. Thus, the spherical form of ferritin comprises 24 monomeric, ferritin subunit proteins, and has a capsid-like structure having 432 symmetry.

According to the present invention, a monomeric ferritin subunit of the present invention is a full length, single polypeptide of a ferritin protein, or any portion thereof, which is capable of directing self-assembly of monomeric ferritin subunits into the spherical form of the protein. Amino acid sequences from monomeric ferritin subunits of any known ferritin protein can be used to produce fusion proteins of the present invention, so long as the monomeric ferritin subunit is capable of directing self-assembly of the fusion protein into a nanoparticle displaying immunogenic portions from influenza virus HA proteins on its surface. In one embodiment, the monomeric ferritin subunit is selected from the group consisting of a bacterial ferritin protein, a plant ferritin protein, an algal ferritin protein, an insect ferritin protein, a fungal ferritin protein and a mammalian ferritin protein. In one embodiment, the monomeric ferritin subunit is from Helicobacter pylori. In one embodiment, the monomeric ferritin subunit is from E. coli. In one embodiment, the monomeric ferritin subunit is bullfrog ferritin. In one embodiment, the monomeric ferritin subunit is a hybrid ferritin protein made by joining amino acid sequences from more than one ferritin proteins selected from the group consisting of H. pylori ferritin, E. coli ferritin and bullfrog ferritin. Amino acid sequences from representative ferritin proteins of the present invention are disclosed herein as SEQ ID NO:64 (H. pylori ferritin), SEQ ID NO:66 (E. coli ferritin), SEQ ID NO:70 (bullfrog ferritin). Examples of representative hybrid ferritin proteins of the present invention include SEQ ID NO:73 (H. pylori ferritin-bullfrog ferritin fusion) and SEQ ID NO:76 (E. coli ferritin-bullfrog ferritin fusion. In one embodiment, nanoparticles of the present invention contain fusion proteins comprising an amino acid sequence selected from the group consisting of SEQ ID NO:64, SEQ ID NO:67, SEQ ID NO:70, SEQ ID NO:73 and SEQ ID NO:76.

In one embodiment, the self-assembly protein is encapsulin. According to the present invention, a monomeric encapsulin subunit of the present invention is a full length, single polypeptide of an encapsulin protein, or any portion thereof, which is capable of directing self-assembly of monomeric encapsulin subunits into a nanoparticle. Amino acid sequences from monomeric encapsulin subunits of any known encapsulin protein can be used to produce fusion proteins of the present invention, so long as the monomeric encapsulin subunit is capable of directing self-assembly of the fusion protein into a nanoparticle displaying immunogenic portions from influenza virus HA proteins on its surface. The amino acid sequence of a representative encapsulin protein is disclosed herein as SEQ ID NO:79. The spherical form of encapsulin comprises 60 monomeric encapsulin subunit proteins.

In one embodiment, the self-assembly protein is artificially designed Salmonella enteritis 03-33 subunit protein. According to the present invention, a monomeric 03-33 subunit of the present invention is a full length, single polypeptide of an 03-33 protein, or any portion thereof, which is capable of directing self-assembly of monomeric 03-33 subunits into a nanoparticle. Amino acid sequences from monomeric 03-33 subunits of any known 03-33 protein can be used to produce fusion proteins of the present invention, so long as the monomeric 03-33 subunit is capable of directing self-assembly of the fusion protein into a nanoparticle displaying immunogenic portions from influenza virus HA proteins on its surface. The amino acid sequence of a representative 03-33 protein is disclosed herein as SEQ ID NO:82.

In one embodiment, the self-assembly protein is sulfur oxygenase reductase (SOR). According to the present invention, a monomeric SOR subunit of the present invention is a full length, single polypeptide of an SOR protein, or any portion thereof, which is capable of directing self-assembly of monomeric SOR subunits into a nanoparticle. Amino acid sequences from monomeric SOR subunits of any known SOR protein can be used to produce fusion proteins of the present invention, so long as the monomeric SOR subunit is capable of directing self-assembly of the fusion protein into a nanoparticle displaying immunogenic portions from influenza virus HA proteins on its surface. The amino acid sequence of a representative SOR protein is disclosed herein as SEQ ID NO:85. The spherical form of SOR comprises 24 monomeric SOR subunit proteins.

In one embodiment, the self-assembly protein is lumazine synthase (LS). According to the present invention, a monomeric LS subunit of the present invention is a full length, single polypeptide of an LS protein, or any portion thereof, which is capable of directing self-assembly of monomeric LS subunits into a nanoparticle. Amino acid sequences from monomeric LS subunits of any known LS protein can be used to produce fusion proteins of the present invention, so long as the monomeric LS subunit is capable of directing self-assembly of the fusion protein into a nanoparticle displaying immunogenic portions from influenza virus HA proteins on its surface. The amino acid sequence of a representative LS protein is disclosed herein as SEQ ID NO:88. The spherical form of LS comprises a 60 monomeric subunit capsid comprising 12 pentameric units.

In one embodiment, the self-assembly protein is pyruvate dehydrogenase complex (PDC) dihydrolipoamide acetyltransferase (E2p). According to the present invention, a monomeric E2p subunit of the present invention is a full length, single polypeptide of an E2p protein, or any portion thereof, which is capable of directing self-assembly of monomeric E2p subunits into a nanoparticle. Amino acid sequences from monomeric E2p subunits of any known E2p protein can be used to produce fusion proteins of the present invention, so long as the monomeric E2p subunit is capable of directing self-assembly of the fusion protein into a nanoparticle displaying immunogenic portions from influenza virus HA proteins on its surface. The amino acid sequence of a representative E2p protein is disclosed herein as SEQ ID NO:91. In one embodiment, the nanoparticles comprise self-assembly proteins from Chikungunya virus. In particular, the nanoparticles comprises one or more structural proteins (e.g., capsid, E1, E2 an E3) from Chikungunya virus (CHKV). Methods of forming nanoparticles from CHKV are disclosed herein and are also taught in U.S. patent application Ser. No. 13/131,287, which is incorporated herein in its entirety by reference. According to the present invention, CHKV structural proteins are full length, single polypeptides of CHKV envelope proteins, or any portion thereof, which are capable of directing self-assembly of monomeric structural proteins into a nanoparticle. Amino acid sequences of structural proteins from any known CHKV virus can be used to produce fusion proteins of the present invention, so long as the amino acid sequences are capable of directing self-assembly of the fusion protein into a nanoparticle displaying immunogenic portions from an influenza virus HA protein on its surface. It is understood by those skilled in the art that CHKV proteins are expressed as a polyprotein, which is subsequently cleaved into individual proteins. The amino acid sequence of a representative CHKV polyprotein is disclosed herein as SEQ ID NO:94. It should be further understood that the amino acid sequences of immunogenic portions can be inserted into the polyprotein such upon cleavage of the polyprotein and formation of the virus-like particle, the immunogenic portions are properly folded and displayed on the surface of the nanoparticle.

HA-SA fusion proteins of the present invention need not comprise the full-length sequence of a monomeric subunit polypeptide of a self-assembly protein. Portions, or regions, of the monomeric SA subunit protein can be utilized so long as the portion comprises an amino acid sequence that directs self-assembly of the HA-SA fusion protein into a nanoparticle. One example of such a portion is located between amino acids 5 and 167 of the Helicobacter pylori ferritin protein (SEQ ID NO:64). More specific regions of the ferritin protein are described in Zhang, Y. Self-Assembly in the Ferritin Nano-Cage Protein Super Family. 2011, Int. J. Mol. Sci., 12, 5406-5421, which is incorporated herein by reference in its entirety.

One embodiment of the present invention is a nanoparticle comprising a heterogeneous population of HA-SA fusion proteins, wherein each HA-SA fusion protein comprises at least one immunogenic portion an influenza virus HA protein, joined to at least 25 contiguous amino acids, at least 50 contiguous amino acids, at least 75 contiguous amino acids, at least 100 contiguous amino acids, or at least 150 contiguous amino acids from a protein selected from the group consisting of ferritin, encapsulin, sulfur oxygenase reductase, lumazine synthase and pyruvate dehydrogenase complex (PDC) dihydrolipoamide acetyltransferase (E2), wherein the HA-SA fusion protein is capable of being assembled into a nanoparticle. One embodiment of the present invention is a nanoparticle comprising a heterogeneous population of HA-SA fusion proteins, wherein each HA-SA fusion protein comprises at least one immunogenic portion of an influenza virus HA protein joined to at least 25 contiguous amino acids, at least 50 contiguous amino acids, at least 75 contiguous amino acids, at least 100 contiguous amino acids, or at least 150 contiguous amino acids from an amino acid sequence selected from the group consisting of SEQ ID NO:64, SEQ ID NO:67, SEQ ID NO:70, SEQ ID NO:73, SEQ ID NO:76, SEQ ID NO:79, SEQ ID NO:82, SEQ ID NO:85, SEQ ID NO:88, SEQ ID NO:91 and SEQ ID NO:94, wherein the HA-SA fusion protein is capable of being assembled into a nanoparticle. One embodiment of the present invention is nanoparticle comprising a heterogeneous population of HA-SA fusion proteins, wherein each fusion protein comprises at least one immunogenic portion of an influenza virus HA protein joined to at least 25 contiguous amino acids, at least 50 contiguous amino acids, at least 75 contiguous amino acids, at least 100 contiguous amino acids, or at least 150 contiguous amino acids from a region of a ferritin protein comprising amino acid residues 5-167 of SEQ ID NO:64, wherein the HA-SA fusion protein is capable of being assembled into a nanoparticle.

As has been previously discussed, it is well-known in the art that some variations can be made in the amino acid sequence of a protein without affecting the activity of that protein. Such variations include insertion of amino acid residues, deletions of amino acid residues, and substitutions of amino acid residues. Thus, in one embodiment, the sequence of a SA protein subunit is divergent enough from the sequence of a SA protein subunit found in nature, such that when the variant SA protein subunit is introduced into an animal, such as a mouse, it does not result in the production of antibodies that react with the natural SA protein. According to the present invention, such a monomeric subunit is referred to as immunogenically neutral. One embodiment of the present invention is a nanoparticle comprising a heterogeneous population of HA-SA fusion proteins, wherein each fusion protein comprises at least one immunogenic portion from an influenza virus HA protein joined to an amino acid sequence at least 80%, at least 85%, at least 90%, at least 95%, or at least 97% identical to the amino acid sequence of a monomeric SA protein subunit protein capable of self-assembling into a nanoparticle, wherein the HA-SA fusion protein is capable of self-assembling into nanoparticles. In one embodiment, the HA-SA fusion protein comprises a polypeptide sequence identical in sequence to a monomeric SA protein subunit selected from the group consisting of ferritin, encapsulin, sulfur oxygenase reductase, lumazine synthase, pyruvate dehydrogenase complex (PDC) dihydrolipoamide acetyltransferase (E2) and the structural proteins of CHKV. One embodiment of the present invention is a nanoparticle comprising a heterogeneous population of HA-SA fusion proteins, wherein each fusion protein comprises at least one influenza virus HA protein immunogenic portion of the present invention joined to an amino acid sequence at least 80%, at least 85%, at least 90%, at least 95%, or at least 97% identical to the amino acid sequence of a monomeric SA protein subunit selected from the group consisting of ferritin, encapsulin, sulfur oxygenase reductase, lumazine synthase, pyruvate dehydrogenase complex (PDC) dihydrolipoamide acetyltransferase (E2) and the envelope proteins of CHKV, wherein the HA-SA fusion protein is capable of self-assembling into nanoparticles. One embodiment of the present invention is a nanoparticle comprising a heterogeneous population of HA-SA fusion proteins, wherein each fusion protein comprises at least one immunogenic portion of an influenza HA protein of the present invention joined to an amino acid sequence at least 80%, at least 85%, at least 90%, at least 95%, and at least 97% identical to a sequence selected from the group consisting of SEQ ID NO:64, SEQ ID NO:67, SEQ ID NO:70, SEQ ID NO:73, SEQ ID NO:76, SEQ ID NO: 79, SEQ ID NO:82, SEQ ID NO:85, SEQ ID NO:88, SEQ ID NO:91 and SEQ ID NO:94, wherein the HA-SA fusion protein is capable of self-assembling into nanoparticles.

In some embodiments of the present invention, the immunogenic portion of an influenza virus HA protein and the amino acid sequence of the SA protein may be joined directly to one another. In other embodiments, it may be necessary to employ linkers (also referred to as a spacer sequences) so that the various domains are in the proper special orientation. The linker sequence is designed to position the immunogenic portion of the influenza virus HA protein in such a way to that it maintains the ability to elicit an immune response to the influenza virus. Linker sequences of the present invention comprise amino acids. Preferable amino acids to use are those having small side chains and/or those which are not charged. Such amino acids are less likely to interfere with proper folding and activity of the fusion protein. Accordingly, preferred amino acids to use in linker sequences, either alone or in combination are serine, glycine and alanine. Examples of such linker sequences include, but are not limited to, SG, SGG, GSG, GG and GGSGG (SEQ ID NO: 192). Amino acids can be added, subtracted or rearranged as needed. Those skilled in the art are capable of determining appropriate linker sequences for proteins of the present invention.

In addition to linker sequences, fusion proteins of the present invention can also comprise other heterologous amino acid sequences. For example, fusion proteins may comprise signal sequences that direct the fusion protein into the proper cellular pathway. For example, a signal sequence may direct the protein into the ER-golgi complex so that it is properly glycosylated and secreted. Any signal sequence can be used as long as it directs the fusion protein in the desired manner. Examples of signal sequences useful for preparing fusion proteins of the present invention include, but are not limited to, the signal sequence from bovine prolactin, the human CD5 signal sequence and the CHIKV signal sequence.

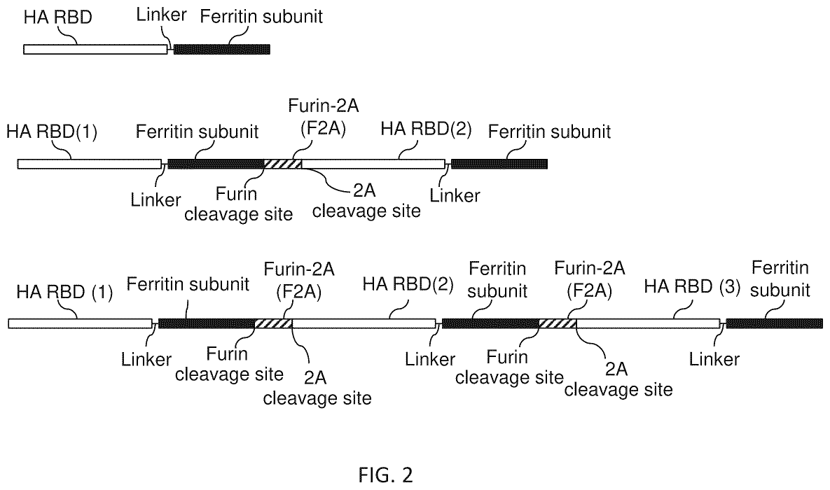

Fusion proteins of the present invention can also contain cleavage sequences. For example, in embodiments in which more than one immunogenic portion from influenza HA proteins are linked together in the fusion protein, enzyme cleavage sites can be included between segments of the fusion protein (e.g., immunogenic portions, SA proteins, linker sequences, etc.) such that upon expression of the protein, the various domains are cleaved from one another. Any cleavage sequence can be used to prepare fusion proteins of the present invention. Examples of such cleavage sequences are furin and 2A cleavage sequences. An exemplary embodiment is illustrated in FIG. 2.

In some embodiments, it may be useful to engineer mutations into the amino acid sequences of fusion proteins of the present invention. For example, it may be useful to alter sites such as enzyme recognition sites or glycosylation sites in the monomeric SA protein, the linker sequence or the immunogenic portions of the influenza HA proteins, in order to give the fusion protein beneficial properties (e.g., stability, solubility, half-life, mask portions of the protein from immune surveillance, avoid steric hinderance, etc). For example, it is known that the monomeric subunit of ferritin is not glycosylated naturally. However, it can be glycosylated if it is expressed as a secreted protein in mammalian or yeast cells. Thus, in one embodiment, potential N-linked glycosylation sites in the amino acid sequences from the monomeric ferritin subunit are mutated so that the mutated ferritin subunit sequences are no longer glycosylated at the mutated site. Examples of useful sites at which to introduce mutations include, but are not limited to, amino acid residues 98 and 264 of influenza virus HA proteins.

One embodiment of the present invention is a fusion protein encoded by a nucleic acid molecule of the present invention. One embodiment of the present invention is a fusion protein encoded by a nucleic acid molecule comprising a nucleic acid sequence at least 80%, at least 85%, at least 90%, at least 95% at least 97%, or which is, identical to a sequence selected from the group consisting of SEQ ID NO:96, SEQ ID NO:99, SEQ ID NO: 102, SEQ ID NO:105, SEQ ID NO: 108, SEQ ID NO: 111, SEQ ID NO: 114, SEQ ID NO: 117, SEQ ID NO:120, SEQ ID NO: 123, SEQ ID NO: 126, SEQ ID NO: 129, SEQ ID NO: 132, SEQ ID NO:135, SEQ ID NO: 138, SEQ ID NO: 141, SEQ ID NO: 144, SEQ ID NO: 147, SEQ ID NO:150, SEQ ID NO: 153, SEQ ID NO: 156, SEQ ID NO: 159, SEQ ID NO: 162, SEQ ID NO:165, SEQ ID NO: 168, SEQ ID NO: 171, SEQ ID NO: 174, SEQ ID NO: 177, SEQ ID NO:180, SEQ ID NO: 183, SEQ ID NO: 186 and SEQ ID NO:189. One embodiment of the present invention is a fusion protein encoded by a nucleic acid molecule comprising a nucleic acid sequence selected from the group consisting of SEQ ID NO:96, SEQ ID NO:99, SEQ ID NO:102, SEQ ID NO: 105, SEQ ID NO: 108, SEQ ID NO: 111, SEQ ID NO: 114, SEQ ID NO:117, SEQ ID NO: 120, SEQ ID NO: 123, SEQ ID NO: 126, SEQ ID NO: 129, SEQ ID NO:132, SEQ ID NO:135, SEQ ID NO: 138, SEQ ID NO: 141, SEQ ID NO: 144, SEQ ID NO:147, SEQ ID NO:150, SEQ ID NO: 153, SEQ ID NO: 156, SEQ ID NO: 159, SEQ ID NO:162, SEQ ID NO: 165, SEQ ID NO: 168, SEQ ID NO:171, SEQ ID NO:174, SEQ ID NO:177, SEQ ID NO: 180, SEQ ID NO: 183, SEQ ID NO: 186 and SEQ ID NO: 189. One embodiment of the present invention is a fusion protein encoded by a nucleic acid molecule consisting of a nucleic acid sequence selected from the group consisting of SEQ ID NO:96, SEQ ID NO:99, SEQ ID NO: 102, SEQ ID NO: 105, SEQ ID NO: 108, SEQ ID NO: 111, SEQ ID NO:114, SEQ ID NO:117, SEQ ID NO: 120, SEQ ID NO: 123, SEQ ID NO: 126, SEQ ID NO:129, SEQ ID NO: 132, SEQ ID NO: 135, SEQ ID NO: 138, SEQ ID NO: 141, SEQ ID NO:144, SEQ ID NO: 147, SEQ ID NO: 150, SEQ ID NO: 153, SEQ ID NO: 156, SEQ ID NO:159, SEQ ID NO: 162, SEQ ID NO: 165, SEQ ID NO: 168, SEQ ID NO: 171, SEQ ID NO:174, SEQ ID NO: 177, SEQ ID NO: 180, SEQ ID NO: 183, SEQ ID NO: 186 and SEQ ID NO:189.

One embodiment of the present invention is a fusion protein comprising an amino acid sequence at least 80%, at least 85%, at least 90%, at least 95% at least 97%, or which is, identical to an amino acid sequence selected form the group consisting of SEQ ID NO:97, SEQ ID NO:100, SEQ ID NO: 103, SEQ ID NO: 106, SEQ ID NO: 109, SEQ ID NO:112, SEQ ID NO:115, SEQ ID NO:118, SEQ ID NO: 121, SEQ ID NO: 124, SEQ ID NO: 127, SEQ ID NO:130, SEQ ID NO: 133, SEQ ID NO: 136, SEQ ID NO: 139, SEQ ID NO: 142, SEQ ID NO:145, SEQ ID NO:148, SEQ ID NO:151, SEQ ID NO: 154, SEQ ID NO: 157, SEQ ID NO: 160, SEQ ID NO: 163, SEQ ID NO: 166, SEQ ID NO: 169, SEQ ID NO: 172, SEQ ID NO:175, SEQ ID NO: 178, SEQ ID NO: 181, SEQ ID NO: 184, SEQ ID NO: 187 and SEQ ID NO:190. One embodiment of the present invention is a fusion protein consisting of an amino acid sequence selected form the group consisting of SEQ ID NO:97, SEQ ID NO: 100, SEQ ID NO:103, SEQ ID NO: 106, SEQ ID NO: 109, SEQ ID NO: 112, SEQ ID NO:115, SEQ ID NO:118, SEQ ID NO:121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO:148, SEQ ID NO: 151, SEQ ID NO: 154, SEQ ID NO: 157, SEQ ID NO: 160, SEQ ID NO:163, SEQ ID NO: 166, SEQ ID NO: 169, SEQ ID NO: 172, SEQ ID NO: 175, SEQ ID NO:178, SEQ ID NO: 181, SEQ ID NO: 184, SEQ ID NO: 187 and SEQ ID NO: 190.

Fusion proteins of the present invention are encoded by nucleic acid molecules of the present invention. In addition, they are expressed by nucleic acid constructs of the present invention. As used herein a nucleic acid construct is a recombinant expression vector, i.e., a vector linked to a nucleic acid molecule encoding a protein such that the nucleic acid molecule can effect expression of the protein when the nucleic acid construct is administered to, for example, a subject or an organ, tissue or cell. The vector also enables transport of the nucleic acid molecule to a cell within an environment, such as, but not limited to, an organism, tissue, or cell culture. A nucleic acid construct of the present disclosure is produced by human intervention. The nucleic acid construct can be DNA, RNA or variants thereof. The vector can be a DNA plasmid, an mRNA, a viral vector, or other vector. In one embodiment, a vector can be a cytomegalovirus (CMV), retrovirus, adenovirus, adeno-associated virus, herpes virus, vaccinia virus, poliovirus, sindbis virus, or any other DNA or RNA virus vector. In one embodiment, a vector can be a pseudotyped lentiviral or retroviral vector. In one embodiment, a vector can be a DNA plasmid. In one embodiment, a vector can be a DNA plasmid comprising viral components and plasmid components to enable nucleic acid molecule delivery and expression. Methods for the construction of nucleic acid constructs of the present disclosure are well known. See, for example, Molecular Cloning: a Laboratory Manual, 3^{rd} edition, Sambrook et al. 2001 Cold Spring Harbor Laboratory Press, and Current Protocols in Molecular Biology, Ausubel et al. eds., John Wiley & Sons, 1994. In one embodiment, the vector is a DNA plasmid, such as a CMV/R plasmid such as CMV/R or CMV/R 8 KB (also referred to herein as CMV/R 8κb). Examples of CMV/R and CMV/R 8 κb are provided herein. CMV/R is also described in U.S. Pat. No. 7,094,598 B2, issued Aug. 22, 2006.

As used herein, a nucleic acid molecule comprises a nucleic acid sequence that encodes an HA-SA fusion protein of the present invention. A nucleic acid molecule can be produced recombinantly, synthetically, or by a combination of recombinant and synthetic procedures. A nucleic acid molecule of the disclosure can have a wild-type nucleic acid sequence or a codon-modified nucleic acid sequence to, for example, incorporate codons better recognized by the human translation system. In one embodiment, a nucleic acid molecule can be genetically-engineered to introduce, or eliminate, codons encoding different amino acids, such as to introduce codons that encode an N-linked glycosylation site. Methods to produce nucleic acid molecules of the disclosure are known in the art, particularly once the nucleic acid sequence is know. It is to be appreciated that a nucleic acid construct can comprise one nucleic acid molecule or more than one nucleic acid molecule. It is also to be appreciated that a nucleic acid molecule can encode one protein or more than one protein.

One embodiment of the present invention is a nucleic acid molecule comprising a nucleic acid sequence encoding a fusion protein of the present invention. One embodiment of the present invention is a nucleic acid molecule comprising a nucleic acid sequence encoding a fusion protein that comprises a monomeric self-assembly subunit protein joined to one or more immunogenic portions of one or more influenza hemagglutinin proteins. One embodiment of the present invention is a nucleic acid molecule comprising a nucleic acid sequence encoding a fusion protein comprising one or more amino acid sequence at least 80%, at least 85%, at least 90%, at least 95% at least 97%, or which are, identical to one or more immunogenic portions from one or more influenza hemagglutinin proteins of the present invention, wherein the fusion protein is capable of forming a nanoparticle displaying the immunogenic portions on its surface. In a further embodiment, a nucleic acid molecule of the present invention comprises a nucleic acid sequence encoding a fusion protein comprising an amino acid sequence at least 80%, at least 85%, at least 90%, at least 95% at least 97%, or which is, identical to the amino acid sequence of a monomeric self-assembly subunit protein of the present invention, wherein the fusion protein is capable of forming a nanoparticle displaying the immunogenic portions on its surface. In one embodiment, a nucleic acid molecule of the present invention comprises a nucleic acid sequence encoding a fusion protein comprising i) one or more amino acid sequence at least 80%, at least 85%, at least 90%, at least 95% at least 97%, or which are, identical to one or more immunogenic portions from one or more influenza hemagglutinin proteins of the present invention; and, ii) an amino acid sequence at least 80%, at least 85%, at least 90%, at least 95% at least 97%, or which is, identical to the amino acid sequence of a monomeric self-assembly subunit protein of the present invention, wherein the fusion protein is capable of forming a nanoparticle displaying the immunogenic portions on its surface. One embodiment of the present invention is a nucleic acid molecule comprising a nucleic acid sequence encoding a fusion protein comprising an amino acid sequence at least 80%, at least 85%, at least 90%, at least 95% at least 97%, or which is, identical to an amino acid sequence selected form the group consisting of SEQ ID NO:97, SEQ ID NO: 100, SEQ ID NO: 103, SEQ ID NO: 106, SEQ ID NO:109, SEQ ID NO:112, SEQ ID NO:115, SEQ ID NO: 118, SEQ ID NO: 121, SEQ ID NO:124, SEQ ID NO: 127, SEQ ID NO: 130, SEQ ID NO: 133, SEQ ID NO: 136, SEQ ID NO:139, SEQ ID NO: 142, SEQ ID NO: 145, SEQ ID NO: 148, SEQ ID NO: 151, SEQ ID NO:154, SEQ ID NO: 157, SEQ ID NO: 160, SEQ ID NO: 163, SEQ ID NO: 166, SEQ ID NO:169, SEQ ID NO: 172, SEQ ID NO: 175, SEQ ID NO: 178, SEQ ID NO: 181, SEQ ID NO:184, SEQ ID NO:187 and SEQ ID NO:190. One embodiment of the present invention is a nucleic acid molecule comprising a nucleic acid sequence encoding a fusion protein comprising an amino acid sequence selected form the group consisting of SEQ ID NO:97, SEQ ID NO:100, SEQ ID NO: 103, SEQ ID NO: 106, SEQ ID NO: 109, SEQ ID NO: 112, SEQ ID NO: 115, SEQ ID NO:118, SEQ ID NO: 121, SEQ ID NO:124, SEQ ID NO:127, SEQ ID NO:130, SEQ ID NO:133, SEQ ID NO: 136, SEQ ID NO: 139, SEQ ID NO: 142, SEQ ID NO: 145, SEQ ID NO:148, SEQ ID NO: 151, SEQ ID NO: 154, SEQ ID NO: 157, SEQ ID NO: 160, SEQ ID NO:163, SEQ ID NO: 166, SEQ ID NO: 169, SEQ ID NO: 172, SEQ ID NO: 175, SEQ ID NO:178, SEQ ID NO: 181, SEQ ID NO: 184, SEQ ID NO: 187 and SEQ ID NO:190. One embodiment of the present invention is a nucleic acid molecule comprising a nucleic acid sequence at least 80%, at least 85%, at least 90%, at least 95% at least 97%, or which is, identical to a sequence selected form the group consisting of SEQ ID NO:96, SEQ ID NO:98, SEQ ID NO:99, SEQ ID NO: 101, SEQ ID NO: 102, SEQ ID NO: 104, SEQ ID NO: 105, SEQ ID NO:107, SEQ ID NO: 108, SEQ ID NO: 110, SEQ ID NO: 111, SEQ ID NO: 113, SEQ ID NO:114, SEQ ID NO: 116, SEQ ID NO:117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO:135, SEQ ID NO:137, SEQ ID NO: 138, SEQ ID NO: 140, SEQ ID NO:141, SEQ ID NO: 143, SEQ ID NO:144, SEQ ID NO: 146, SEQ ID NO: 147, SEQ ID NO: 149, SEQ ID NO: 150, SEQ ID NO:152, SEQ ID NO: 153, SEQ ID NO: 155, SEQ ID NO: 156, SEQ ID NO: 158, SEQ ID NO:159, SEQ ID NO: 161, SEQ ID NO: 162, SEQ ID NO: 164, SEQ ID NO: 165, SEQ ID NO: 167, SEQ ID NO: 168, SEQ ID NO: 170, SEQ ID NO: 171, SEQ ID NO: 173, SEQ ID NO:174, SEQ ID NO: 176, SEQ ID NO: 177, SEQ ID NO: 179, SEQ ID NO: 180, SEQ ID NO:182, SEQ ID NO:183, SEQ ID NO: 185, SEQ ID NO: 186, SEQ ID NO: 188, SEQ ID NO:189 and SEQ ID NO: 191. One embodiment of the present invention is a nucleic acid molecule comprising a nucleic acid sequence selected from the group consisting of SEQ ID NO:96, SEQ ID NO:98, SEQ ID NO:99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO:113, SEQ ID NO: 114, SEQ ID NO: 116, SEQ ID NO: 117, SEQ ID NO:119, SEQ ID NO:120, SEQ ID NO: 122, SEQ ID NO:123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO:135, SEQ ID NO:137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158, SEQ ID NO: 159, SEQ ID NO: 161, SEQ ID NO: 162, SEQ ID NO: 164, SEQ ID NO:165, SEQ ID NO: 167, SEQ ID NO: 168, SEQ ID NO: 170, SEQ ID NO: 171, SEQ ID NO:173, SEQ ID NO:174, SEQ ID NO: 176, SEQ ID NO: 177, SEQ ID NO: 179, SEQ ID NO:180, SEQ ID NO: 182, SEQ ID NO: 183, SEQ ID NO: 185, SEQ ID NO: 186, SEQ ID NO:188, SEQ ID NO: 189 and SEQ ID NO:191. One embodiment of the present invention is a nucleic acid molecule consisting of a nucleic acid sequence selected from the group consisting of SEQ ID NO:96, SEQ ID NO:98, SEQ ID NO:99, SEQ ID NO: 101, SEQ ID NO: 102, SEQ ID NO:104, SEQ ID NO: 105, SEQ ID NO: 107, SEQ ID NO: 108, SEQ ID NO:110, SEQ ID NO: 111, SEQ ID NO: 113, SEQ ID NO: 114, SEQ ID NO: 116, SEQ ID NO:117, SEQ ID NO:119, SEQ ID NO: 120, SEQ ID NO: 122, SEQ ID NO: 123, SEQ ID NO: 125, SEQ ID NO:126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO:141, SEQ ID NO: 143, SEQ ID NO: 144, SEQ ID NO: 146, SEQ ID NO: 147, SEQ ID NO:149, SEQ ID NO: 150, SEQ ID NO: 152, SEQ ID NO: 153, SEQ ID NO: 155, SEQ ID NO: 156, SEQ ID NO: 158, SEQ ID NO: 159, SEQ ID NO: 161, SEQ ID NO: 162, SEQ ID NO:164, SEQ ID NO:165, SEQ ID NO: 167, SEQ ID NO: 168, SEQ ID NO: 170, SEQ ID NO:171, SEQ ID NO: 173, SEQ ID NO: 174, SEQ ID NO: 176, SEQ ID NO: 177, SEQ ID NO:179, SEQ ID NO: 180, SEQ ID NO: 182, SEQ ID NO: 183, SEQ ID NO: 185, SEQ ID NO:186, SEQ ID NO: 188, SEQ ID NO: 189 and SEQ ID NO:191.

As has been discussed, nanoparticles of the present invention comprise populations of fusion proteins which are heterogeneous due to at least two fusion proteins in the population differing in their amino acid sequences by at least one amino acid. It will be appreciated by those skilled in the art that, as described hereto, a heterogeneous population of fusion proteins can be due to the aforementioned amino acid difference being at any location in the fusion protein, including in the SA portion of the protein. However, preferred nanoparticles of the present invention are those in which a single nanoparticle is capable of eliciting an immune response to more than one Type, sub-type or strain of influenza virus. Consequently, preferred nanoparticles are those comprising a heterogeneous population of fusion proteins, wherein at least two fusion proteins in the heterogeneous population differ in the sequences of their immunogenic portions by at least one amino acid. It should be understood that fusion proteins of preferred nanoparticles are not excluded from having sequences differences in regions other than the immunogenic portion. However, in order to elicit an immune response against more than one Type, Group, sub-type or strain of influenza virus, preferred nanoparticles comprise at least two fusion proteins that differ by at least one amino acid residue in the their immunogenic portions. It will be understood by those skilled in the art that differences in the amino acid sequences of the immunogenic portion of two fusion proteins may or may not cause the two different immunogenic portions (i.e., the two species of immunogenic portions) to be recognized by two different receptors (e.g., B-cell, T-cell, etc). Such differences, or lack thereof, in recognition depend on such things as, for example, the differences in properties between the corresponding amino acid residues in the immunogenic portions and whether or not the locations at which the sequences differ (i.e., the amino acid residue) are part of the recognized epitope. In preferred embodiments, the heterogeneous population comprises at least two species of fusion proteins, wherein the immunogenic portions of each of the species is recognized by the same B-cell receptor, T-cell receptor and/or antibody. Thus, in one embodiment, a nanoparticle of the present invention elicits a cross-reactive immune response (an immune response against more than one Type, subtype or strain of influenza virus).

It should be understood that the number of immunogenic regions displayed by nanoparticles of the present invention is only limited by the number of fusion proteins that make up the nanoparticle, which itself is determined by the SA protein used to construct the fusion proteins. For example, ferritin forms a nanoparticle consisting of 24 monomeric, ferritin subunit proteins. Thus, ferritin-based nanoparticle of the present invention can comprise a maximum of 24 fusion proteins and thus, can display a maximum of 24 different immunogenic portions. Similarly, encapsulin proteins from Thermotoga maritima form nanoparticles having 60 subunits. Thus, encapsulin-based nanoparticle of the present invention can display a maximum of 60 different immunogenic portions. Likewise, structural proteins from CHIKV form virus-like particles having 240 envelope E2 subunits. Thus CHIKV-based virus-like particle of the present invention can display a maximum of 240 different immunogenic portions. Those skilled in the art will understand that such calculations assume each fusion protein comprises a single immunogenic portion. Nanoparticles displaying higher numbers of immunogenic portions could of course be constructed using fusion proteins comprising two or more immunogenic portions. An example of a fusion protein comprising multiple epitopes is illustrated in FIG. 2. In one embodiment, the nanoparticles comprises a heterogeneous population of fusion proteins, wherein each fusion protein comprises a single immunogenic portion of an influenza HA protein. In one embodiment, the nanoparticles comprises a heterogeneous population of fusion proteins, wherein each fusion protein comprises multiple immunogenic portions from one or more influenza HA proteins. In one embodiment, the nanoparticles comprises a heterogeneous population of fusion proteins, wherein each fusion protein comprises at least 2, at least 3, at least 4 or at least 5 immunogenic portions from one or more influenza HA proteins.

In one embodiment, a nanoparticle of the present invention comprises between 2 and 240 species of fusion proteins, wherein each species differs from every other species, at least in part, by at least one amino acid in change in the sequence of its immunogenic portion. In certain embodiments, a nanoparticle of the present invention comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 3, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230 or at least 240 species of fusion proteins, wherein the species differ from one another, at least in part, by at least one amino acid in their immunogenic portions. In certain embodiments, a nanoparticle of the present invention display at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 3, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59 or at least 60 unique immunogenic portions.

One embodiment of the present invention is a method of producing nanoparticles of the present invention, the method comprising introducing one or more nucleic acid molecules encoding fusion proteins of the present invention into a cell, and incubating the cell under conditions suitable for expressing the encoded proteins and forming nanoparticles. In a further embodiment, the nanoparticles are isolated from the cell into which the nucleic acid molecule was introduced. Methods of isolating nanoparticles are known to those skilled in the art and are also described in U.S. patent application Ser. No. 13/131,287 and International Application No. PCT/US14/60142, both of which are incorporated herein by reference in their entirety. It should be realized by those skilled in the art nanoparticles displaying a heterogeneous population of immunogenic portions on its surface can be produced by either i) introducing more than one nucleic acid molecule into the cell, wherein each nucleic acid molecule encodes a different species of fusion protein; and/or ii) introducing one or more nucleic acid molecule into a cell, wherein at least one nucleic acid molecule encode at least one of the one or more nucleic acid molecules encode a species of fusion protein that differs from a fusion protein encoded by a nucleic acid molecule introduced into the cell. Thus, for example, a nanoparticle comprising a heterogeneous population of fusion proteins can be produced by introducing into a cell a nucleic acid molecule encoding two different species of fusion proteins.

Nanoparticles of the present invention can also be produced by combining expressed and isolated recombinant proteins. Thus, one embodiment of the present invention is a method of producing nanoparticles of the present invention, the method comprising introducing into a cell a nucleic acid molecule encoding a fusion protein of the present invention, incubating the cell under conditions suitable for expressing the protein encoded by the nucleic acid molecule, and isolating the expressed protein. The isolated proteins are then disassembled and combined with one or more heterogeneous species of isolated, disassembled fusion protein (i.e., fusion proteins having a different sequence, especially in their immunogenic portion) such that the mixture of heterogeneous species of fusion proteins reassembles in a nanoparticle comprising a heterogeneous population of fusion proteins, wherein each nanoparticle comprises at least two different species of fusion proteins.

Because nanoparticles of the present invention elicit an immune response to an infectious agent, such as influenza virus, they can be used as vaccines to protect individuals against infection by one or more Types, sub-types, strains and/or species of infectious agent (e.g., influenza virus). Thus, one embodiment of the present invention is a vaccine comprising a nanoparticle of the present invention. Vaccines of the present invention can also contain other components such as adjuvants, buffers and the like. Although any adjuvant can be used, preferred embodiments can contain: chemical adjuvants such as aluminum phosphate, benzyalkonium chloride, ubenimex, and QS21; genetic adjuvants such as the IL-2 gene or fragments thereof, the granulocyte macrophage colony-stimulating factor (GM-CSF) gene or fragments thereof, the IL-18 gene or fragments thereof, the chemokine (C-C motif) ligand 21 (CCL21) gene or fragments thereof, the IL-6 gene or fragments thereof, CpG, LPS, TLR agonists, and other immune stimulatory genes; protein adjuvants such IL-2 or fragments thereof, the granulocyte macrophage colony-stimulating factor (GM-CSF) or fragments thereof, IL-18 or fragments thereof, the chemokine (C-C motif) ligand 21 (CCL21) or fragments thereof, IL-6 or fragments thereof, CpG, LPS, TLR agonists and other immune stimulatory cytokines or fragments thereof; lipid adjuvants such as cationic liposomes, N3 (cationic lipid), monophosphoryl lipid A (MPL1); other adjuvants including cholera toxin, enterotoxin, Fms-like tyrosine kinase-3 ligand (Flt-3L), bupivacaine, marcaine, and levamisole.

One embodiment of the present invention is a method to vaccinate an individual against an infectious agent, the method comprising administering to the individual a nanoparticle of the present invention. One embodiment of the present invention is a method to vaccinate an individual against influenza virus, the method comprising administering to the individual a nanoparticle vaccine of the present invention. In one embodiment, the nanoparticle comprises self-assembling fusion proteins of the present invention, and the nanoparticle displays on its surface a heterogeneous population of immunogenic portions from HA proteins from one or more types, subtypes or strains of influenza virus.

One embodiment of the present invention is a method to vaccinate an individual against infection with influenza virus, the method comprising:
a) obtaining a nanoparticle of the present invention; and,
b) administering the nanoparticle to an individual such that an immune response against an influenza virus is produced. As used herein, an immune response to a vaccine, or nanoparticle, of the present invention is the development in a subject of a humoral and/or a cellular immune response to a hemagglutinin protein present in the vaccine. For purposes of the present invention, a “humoral immune response” refers to an immune response mediated by antibody molecules, including secretory IgA or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4+ and CD8+T-cells.

Thus, an immunological response may be one that stimulates CTLs, and/or the production or activation of helper T-cells. The production of chemokines and/or cytokines may also be stimulated. The vaccine may also elicit an antibody-mediated immune response. Hence, an immunological response may include one or more of the following effects: the production of antibodies (e.g., IgA or IgG) by B-cells; and/or the activation of suppressor, cytotoxic, or helper T-cells and/or T-cells directed specifically to a protein (e.g., hemagglutinin) present in the vaccine. These responses may serve to neutralize infectivity, and/or mediate antibody-complement, or antibody dependent cell cytotoxicity (ADCC) to provide protection to an immunized individual. Such responses can be determined using standard immunoassays and neutralization assays, well known in the art.

As used herein, neutralizing antibodies are antibodies that prevent in infectious agent from replicating and spreading within a host. With regard to influenza virus, neutralizing antibodies prevent influenza virus from completing one round of replication. As defined herein, one round of replication refers the life cycle of the virus, starting with attachment of the virus to a host cell and ending with budding of newly formed virus from the host cell. This life cycle includes, but is not limited to, the steps of attaching to a cell, entering a cell, cleavage and rearrangement of the HA protein, fusion of the viral membrane with the endosomal membrane, release of viral ribonucleoproteins into the cytoplasm, formation of new viral particles and budding of viral particles from the host cell membrane.

In one embodiment, a vaccine or nanoparticle of the present invention elicits broadly neutralizing antibodies. As used herein, broadly neutralizing antibodies are antibodies that neutralize more than one genera, type, subtype, species and/or strain of infectious agent within a taxonomic family. With specific regard to influenza viruses used herein, broadly neutralizing antibodies are antibodies that neutralize more than one type, subtype and/or strain of influenza virus. For example, broadly neutralizing antibodies elicited against an HA protein from a Type A influenza virus may neutralize a Type B or Type C virus. As a further example, broadly neutralizing antibodies elicited against an HA protein from Group 1 influenza virus may neutralize a Group 2 virus. As an additional example, broadly neutralizing antibodies elicited against an HA protein from one sub-type or strain of virus, may neutralize another sub-type or strain of virus. For example, broadly neutralizing antibodies elicited against an HA protein from an H1 influenza virus may neutralize viruses from one or more sub-types selected from the group consisting of H2, H3, H4, H5, H6, H7, H8, H8, H10, H11, H12, H13, H14, H15, H16, H17 or H18.

The terms individual, subject, and patient are well-recognized in the art, and are herein used interchangeably to refer to any human or other animal susceptible to influenza infection. Examples include, but are not limited to, humans and other primates, including non-human primates such as chimpanzees and other apes and monkey species; farm animals such as cattle, sheep, pigs, seals, goats and horses; domestic mammals such as seals, dogs and cats; laboratory animals including rodents such as mice, rats and guinea pigs; birds, including domestic, wild and game birds such as chickens, turkeys and other gallinaceous birds, ducks, geese, and the like. The terms individual, subject, and patient by themselves, do not denote a particular age, sex, race, and the like. Thus, individuals of any age, whether male or female, are intended to be covered by the present disclosure and include, but are not limited to the elderly, adults, children, babies, infants, and toddlers. Likewise, the methods of the present invention can be applied to any race, including, for example, Caucasian (white), African-American (black), Native American, Native Hawaiian, Hispanic, Latino, Asian, and European. An infected subject is a subject that is known to have influenza virus in their body.

Methods of the present invention can be used to vaccinate any individual. Such individual can, but need not, be suspected of having been exposed to an infectious agent, such as influenza virus. Similarly, methods of the present invention can be used to vaccinate an individual known to have been exposed to and infectious agent, such as influenza virus, or a person suspected of, or known to have, having been exposed to an infectious agent, such as influenza virus. As such, methods of the present invention can be used to contain a known, or potential, out break of an infectious agent, such as influenza (e.g., epidemic, pandemic).

One embodiment of the present invention is a method to vaccinate an individual against influenza virus, the method comprising administering a vaccine of the embodiments to an individual in need of such a vaccine, such that an immune response against influenza virus is produced in the individual, wherein the vaccine comprises a nanoparticle comprising self-assembling fusion proteins, wherein the nanoparticle displays on its surface a heterogeneous population of immunogenic portions from HA proteins from one or more Type, Group, sub-type or strain of influenza virus. In one embodiment, the immunogenic portions are from the globular head regions of HA proteins from one or more Type, Group, sub-type or strain of influenza virus. In one embodiment, the immunogenic portions are from the RBDs of HA proteins from one or more Type, group, sub-type or strain of influenza virus.

Another embodiment of the present invention is a method to vaccinate an individual against infection with influenza virus, the method comprising:
a) obtaining a vaccine comprising at least one nanoparticle comprising HA-SA fusion proteins, wherein the fusion proteins comprise an SA protein joined to an immunogenic portion of an influenza HA protein, and wherein the nanoparticle displays on its surface a heterogeneous population of immunogenic portions from HA proteins from one or more Type, Group, sub-type or strain of influenza virus; and,
b) administering the vaccine to an individual such that an immune response against an influenza virus is produced. In one embodiment, the immunogenic portions are from the globular head regions of HA proteins from one or more Types, sub-types or strains of influenza virus. In one embodiment, the immunogenic portions are from the RBDs of HA proteins from one or more Types, sub-types or strains of influenza virus.

Vaccines of the present invention can be used to vaccinate individuals using a prime/boost protocol. Such a protocol is described in U.S. Patent Publication No. 20110177122, which is incorporated herein by reference in its entirety. In such a protocol, a first vaccine composition may be administered to the individual (prime) and then after a period of time, a second vaccine composition may be administered to the individual (boost). Administration of the boosting composition is generally weeks or months after administration of the priming composition, preferably about 2-3 weeks or 4 weeks, or 8 weeks, or 16 weeks, or 20 weeks, or 24 weeks, or 28 weeks, or 32 weeks. In one embodiment, the boosting composition is formulated for administration about 1 week, or 2 weeks, or 3 weeks, or 4 weeks, or 5 weeks, or 6 weeks, or 7 weeks, or 8 weeks, or 9 weeks, or 16 weeks, or 20 weeks, or 24 weeks, or 28 weeks, or 32 weeks after administration of the priming composition. As used herein, a vaccinated subject is a subject that has been administered a vaccine that is intended to provide a protective effect against an influenza virus.

The first and second vaccine compositions can be, but need not be, the same composition. Thus, in one embodiment of the present invention, the step of administering the vaccine comprises administering a first vaccine composition, and then at a later time, administering a second vaccine composition. In one embodiment, the first vaccine composition comprises a nanoparticle of the present invention.

In one embodiment, the individual has been exposed to influenza virus. As used herein, the terms exposed, exposure, and the like, indicate the subject has come in contact with a person of animal that is known to be infected with an influenza virus. Vaccines of the present invention may be administered using techniques well known to those in the art. Techniques for formulation and administration may be found, for example, in “Remington's Pharmaceutical Sciences”, 18th ed., 1990, Mack Publishing Co., Easton, PA. Vaccines may be administered by means including, but not limited to, traditional syringes, needleless injection devices, or microprojectile bombardment gene guns. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections, just to name a few. For injection, the compounds of one embodiment of the invention may be formulated in aqueous solutions, preferably in physiologically compatible buffers such as Hanks' solution, Ringer's solution, or physiological saline buffer.

In one embodiment, vaccines, or nanoparticles, of the present invention can be used to protect an individual against infection by heterologous influenza virus. That is, a vaccine made using hemagglutinin protein from one strain of influenza virus is capable of protecting an individual against infection by different strains of influenza. For example, a vaccine made using hemagglutinin protein from influenza A/New Caledonia/20/1999 (H1N1), A/California/04/2009 (H1N1), A/Singapore/1/1957 (H2N2), A/Hong Kong/1/1968 (H3N2), A/Brisbane/10/2007 (H3N2), A/Indonesia/05/2005 (H5N1), B/Florida/4/2006 (influenza B), A/Perth/16/2009 (H3N2), A/Brisbane/59/2007 (H1N1), B/Brisbane/60/2008 (influenza B), A/Wilson-Smith/33 (H1N1), A/Tientsin/78/77 (H1N1), A/Texas/36/91 (H1N1), A/Singapore/6/86 (H1N1), A/Memphis/39/83 (H1N1), A/Malaysia/54 (H1N1), A/Iowa/43 (H1N1), A/Hong Kong/117/77 (H1N1), A/Fort Monmouth/1/47 (H1N1), A/Brisbane/59/07 (H1N1), A/Baylor/4052/81 (H1N1), A/Albany/4835/48 (H1N1), A/Hong Kong/156/97 (H5N1), A/common magpie/Hong Kong/5052/07 (H5N1), A/chicken/Shanxi/2/06 (H5N1), A/silky chicken/Hong Kong/SF189/01 (H5N1), A/chicken/Henan/16/04 (H5N1), A/Victoria/361/11 (H3N2), B/Massachusetts/2/12 (influenza B), B/Brisbane/60/08 (influenza B) and A/Texas/50/12 (H3N2).

In one embodiment, vaccines, or nanoparticles, of the present invention can be used to protect an individual against infection by an antigenically divergent influenza virus. In this regard, the term antigenically divergent refers to the tendency of a strain of influenza virus to mutate over time, thereby changing the amino acids that are displayed to the immune system. Such mutation over time is also referred to as antigenic drift. Thus, for example, a vaccine made using hemagglutinin protein from an A/New Caledonia/20/1999 (H1N1) strain of influenza virus is capable of protecting an individual against infection by earlier, antigenically divergent New Caledonia strains of influenza, and by evolving (or diverging) influenza strains of the future.

One embodiment of the present invention is a kit for practicing methods of the present invention. Kits can include nanoparticles or vaccines of the present invention as well components for making such nanoparticles and vaccines. As such, kits can include, for example, primers, nucleic acid molecules, expression vectors, DNA constructs encoding proteins of the present invention, cells, buffers, reagents, syringes, and directions for using any of said components. It should be appreciated that a kit may comprise more than one container comprising any of the aforementioned, or related, components. For example, certain parts of the kit may require refrigeration, whereas other parts can be stored at room temperature. Thus, as used herein, a kit comprises components sold in separate containers by one or more entity, with the intention that the components contained therein be used together.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

The inventors have discovered that specific fusion proteins comprising portions of hemagglutinin protein are useful for eliciting a broad immune response against influenza viruses. Each of these embodiments will now be disclosed in detail below.

### EXAMPLES

### Example 1. Production of Heterogeneous Nanoparticles

### A. Gene Synthesis and Vector Construction

All genes used in the study were human codon optimized. The gene encoding Helicobacter pylori-bullfrog hybrid ferritin was constructed by fusing residues 2-9 of bullfrog (Rana catesbeiana) ferritin lower subunit (UniProt: P07797 with a point mutation at residue 8 (N8Q) to abolish a potential N-glycosylation site) to H. pylori nonheme ferritin (UniProt: Q9ZLI1, residues 3-167) with mutations at residue 7 (17E) and residue 19 (N19Q) to make a salt bridge with 6R of bullfrog ferritin and abolish a potential N-glycosylation site, respectively. In some cases, there were extra GS residues at the carboxyl terminus of H. pylori ferritin. The secreted encapsulin gene was constructed by fusing a human CD5 signal to Termotoga maritima encapsulin (UniProt: Q9WZP2, residues 1-264). The genes encoding HA RBD (residues 56-264, H3 numbering system) were synthesized or amplified from appropriate plasmids. In some cases, the Y98F mutation was introduced to abolish sialic acid binding property of HA, and the F/K264A mutation to avoid potential steric crash at the junction between HA RBD and nanoparticle scaffolds. These fragments were fused to downstream of a modified bovine prolactin signal sequence (bPRL: MDSKGSSQKG SRLLLLLVVS NLLLPQGVLA, residues 1-30 of SEQ ID NO: 97) and upstream of the hybrid ferritin with a SG linker to give rise to the HA RBD-ferritin genes. To construct the HA RBD-encapsulin genes, gp350 fragments were fused to downstream of encapsulin with a SG linker. To construct HA RBD-Chikungunya virus-like particle (CHIKVLP), the HA RBD gene fragments (residues 59-264, H3 numbering system) were amplified and inserted in the furin cleavage loop between envelope E3 and E2. To accommodate HA RBD insertion and furin cleavage, there were 3 amino acid deletions in E3 (E3 residues 58-60, SPH) and 4 amino acid deletions in E2 (E2 residues 1-4, STKD). All genes were then cloned into the CMV/R or CMV/R 8κb mammalian expression vector for protein production.

### B. Biosynthesis of Recombinant Proteins and Purification

The expression vectors were transiently transfected into FreeStyle 293F or Expi293F cells (Life Technologies) using 293fectin or ExpiFectamine 293 transfection reagents, respectively (Life Technologies). For co-transfection, equimolar amount of 2-8 different plasmids were mixed (a total DNA amount was constant for all transfections). Four days after transfection, culture supernatants were harvested and cleared. The HA RBD-ferritin and HA RBD-encapsulin nanoparticles were purified by ion exchange chromatography using Q Sepharose HP (GE Healthcare) followed by size exclusion chromatography with a Superose 6 10/300 GL column (GE Healthcare) in PBS. The HA RBD-CHIKVLP were purified by ultracentrifugation using Opriprep (Sigma-Aldrich). Briefly, the cleared culture supernatants were overlaid on 1 ml of Optiprep and spun at 50,000 ref in an SW 32 Ti rotor for 90 min. After the spin, bottom 2 ml was collected, mixed thoroughly to make 1:1 Optiprep/concentrated supernatant mixture, and spun again at 360,000 rcf in an NVT 100 rotor for 3 hours. The band corresponding HA RBD-CHIKVLP was collected and further purified by a Sephacryl S-500 16/60 HR column (GE Healthcare) in PBS.

### C. Electron Microscopy (EM) of Purified Nanoparticles

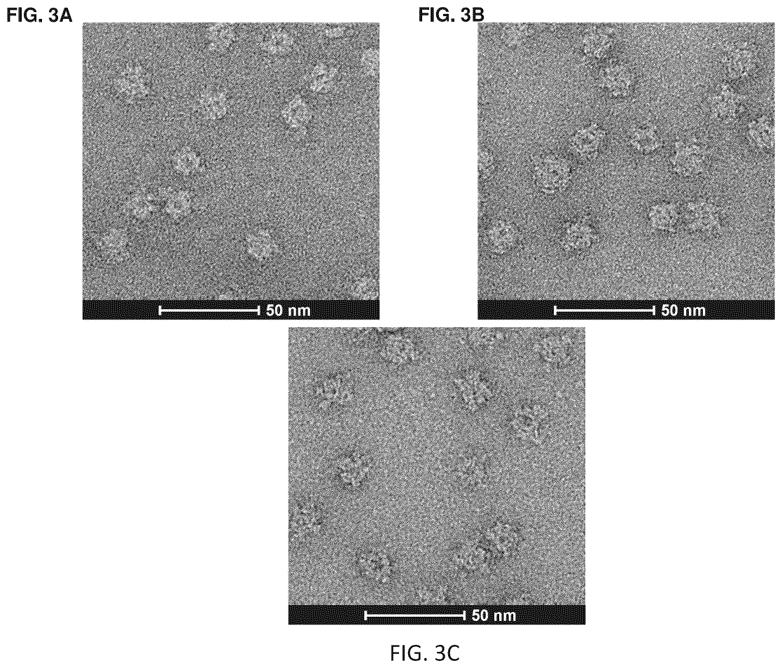

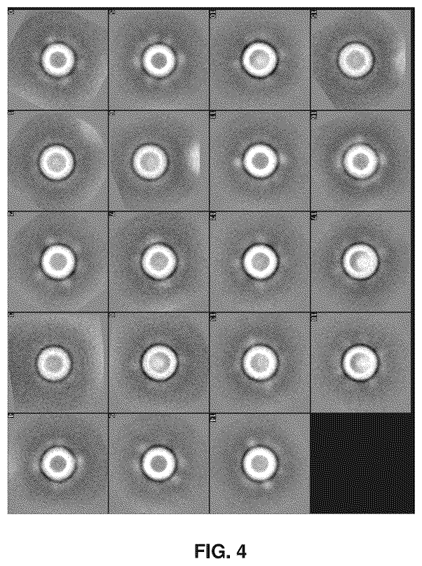

The nanoparticles purified in part (B) were analyzed by negative stain EM. Briefly, samples of about 50 μg ml⁻¹ were adsorbed to freshly glow-discharged carbon-coated grids, rinsed with PBS, and stained with 0.75% uranyl formate solution. Images were recorded on an FEI T20 microscope with an Eagle CCD camera. The results of these analyses are shown in FIGS. 3 and 4.

### Example 2. Immunoprecipitation Analysis of Purified Nanoparticles Expressing Influenza HA Protein RBDs

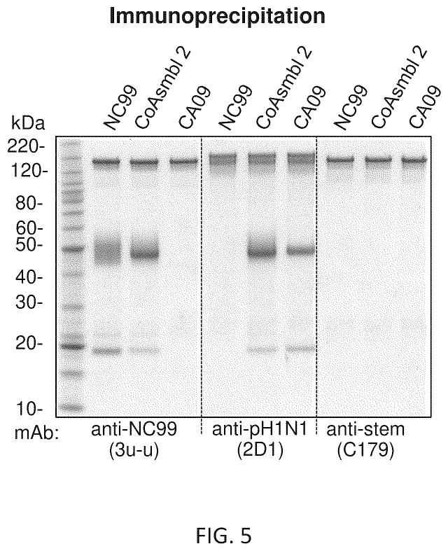

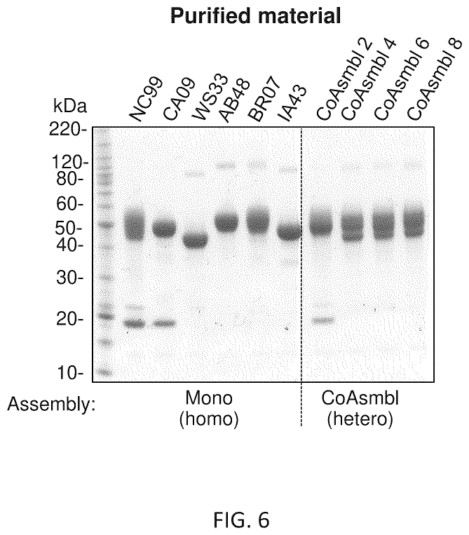

HA RBD-nanoparticles expressing RBDs from NC99, CA09 or both (CoAsmbl 2) were prepared and purified as described in Example 1. Four micrograms of purified RBD-nanoparticles were incubated with 4 μg of either anti-NC99 (3u-u), anti-pandemic HIN1 HA (2D1) or anti-HA stem (C179) monoclonal antibodies for 30 min at room temperature. Immune complexes were then captured using protein G-conjugated magnetic beads, and the complexes washed thoroughly with PBS containing 0.01% Tween 20. The washed pellets were resuspended in 20 μl of Laemmli buffer without reducing agent and analyzed on SDS-PAGE. Five micrograms of each protein were loaded. NC99, A/New Caledonia/20/1999; CA09, A/California/04/2009; WS33, A/Wilson-Smith/1933; AB48, A/Albany/4835/1948; BR07, A/Brisbane/59/2007; IA43, A/Iowa/1943; HK77, A/Hong Kong/117/1977; FM47, A/Fort Monmouth/1/1947. The results of these analyses are shown in FIGS. 5 and 6

### Example 3. Immunization of Mice Using Purified Nanoparticles

The ability of compositions comprising purified monovalent, admixed, or heterogeneous nanoparticles to elicit a neutralizing immune response was tested in mice. Six to eight week old BALB/c mice were divided into 9 groups (N=5). To each group was administered a composition comprising 2 μg of either a) a monovalent (i.e., expresses single HA RBD) nanoparticle, b) a mixture of various monovalent nanoparticle, or c) the indicated, co-assembled nanoparticles, in the presence of Sigma Adjuvant System (SAS) at weeks 0 and 3. The immunogens administered and the dosing schedule is shown below in Table 3.

TABLE 3

| Group¹ | HA RBD² | Dose³ | Adjuvant⁴ | Immunization |
|---|---|---|---|---|
| Mono | NC99 | 2 μg | SAS | Week 0, 3 |
| (NC99) | | | | (20) |
| Mono | CA09 | 2 μg | SAS | Week 0, 3 |
| (CA09) | | | | (20) |
| Admixed 2 | NC99/CA09 | 2 μg | SAS | Week 0, 3 |
| | | total | | (20) |
| Admixed 4 | 2 + WS33/AB48 | 2 μg | SAS | Week 0, 3 |
| | | total | | (20) |
| Admixed 6 | 4 + BR07/IA43 | 2 μg | SAS | Week 0, 3 |
| | | total | | (20) |
| CoAsmbl 2 | NC99/CA09 | 2 μg | SAS | Week 0, 3 |
| | | | | (20) |
| CoAsmbl 4 | 2 + WS33/AB48 | 2 μg | SAS | Week 0, 3 |
| | | | | (20) |
| CoAsmbl 6 | 4 + BR07/IA43 | 2 μg | SAS | Week 0, 3 |
| | | | | (20) |
| CoAsmbl 8 | 6 + HK77/FM47 | 2 μg | SAS | Week 0, 3 |
| | | | | (20) |

¹Balb/c mice (N = 5)
²A/New Caledonia/20/99 (NC99); A/California/04/09 (CA09); A/Wilson-Smith/33 (WS33); A/Albany/4835/48 (AB48); A/Brisbane/59/07 (BR07); A/Iowa/43 (IA43); A/Hong Kong/117/77 (HK77); A/Fort Monmouth/1/47 (FM47)
³Total protein amount per dose
⁴Sigma Adjuvant System (SAS)

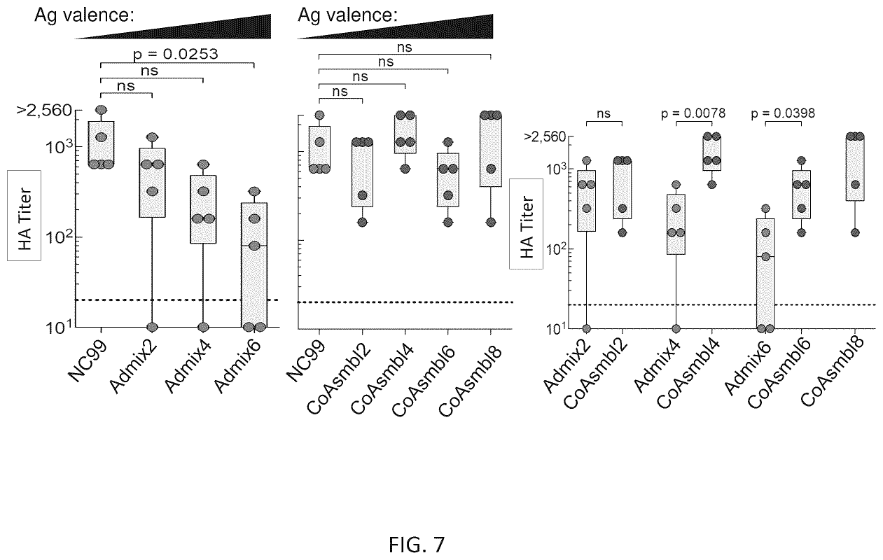

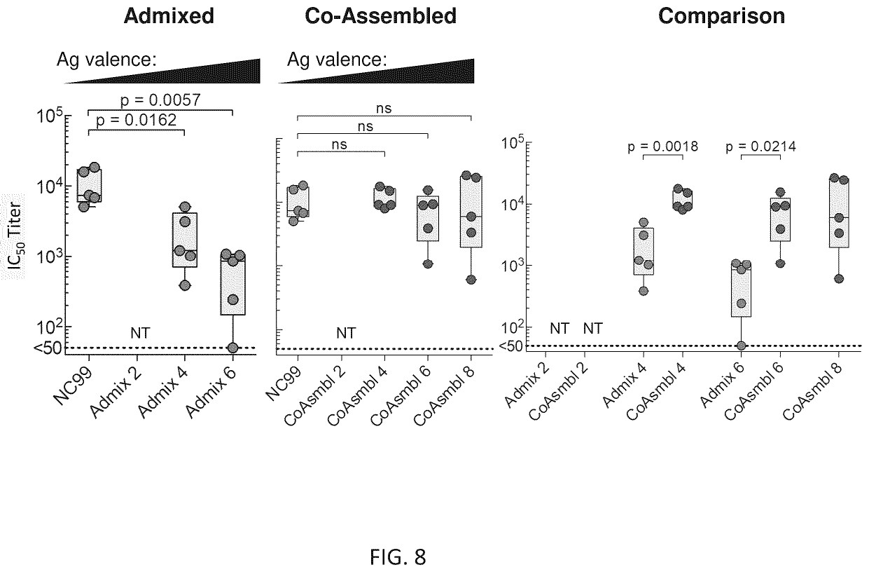

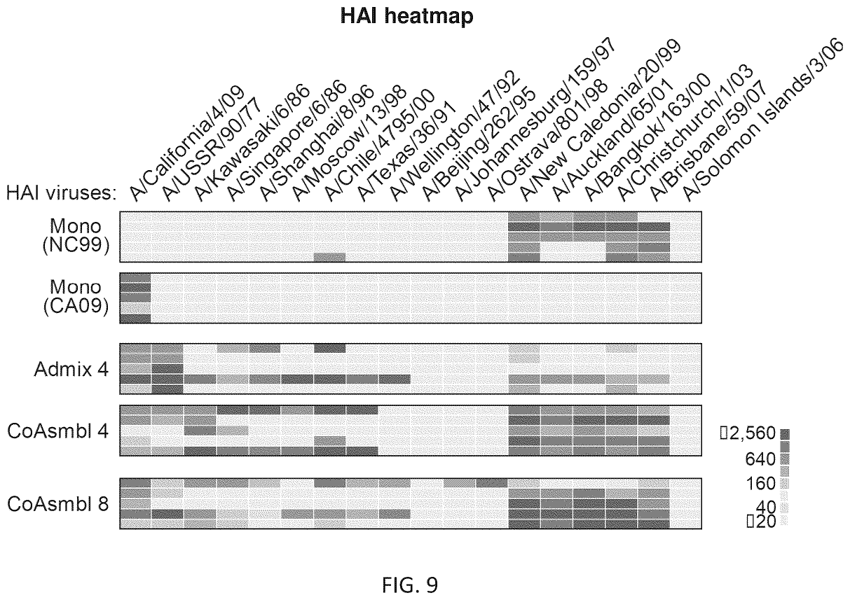

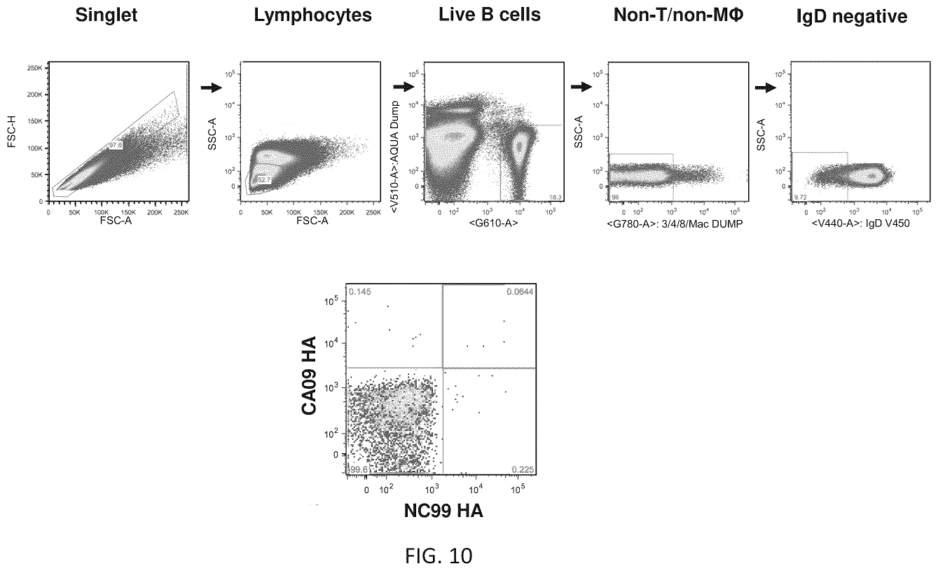

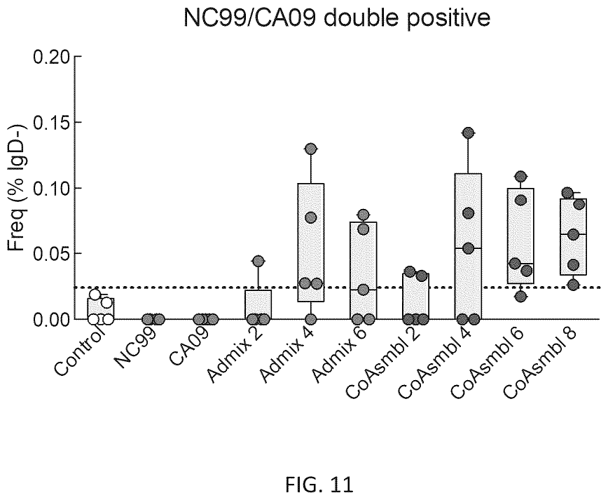

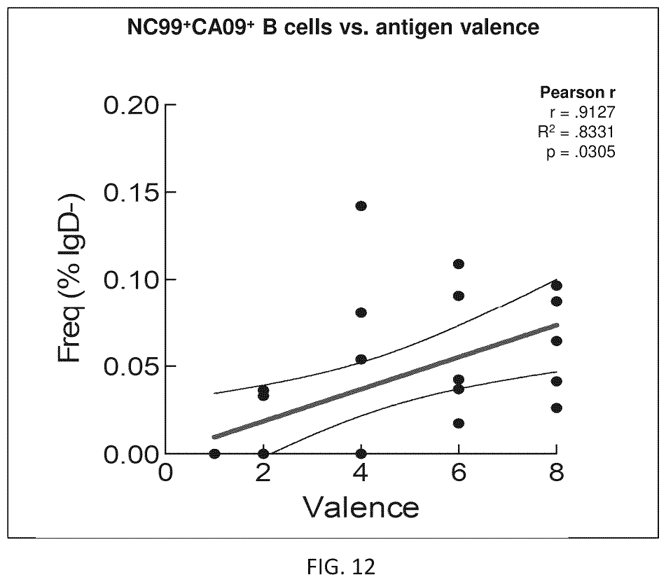

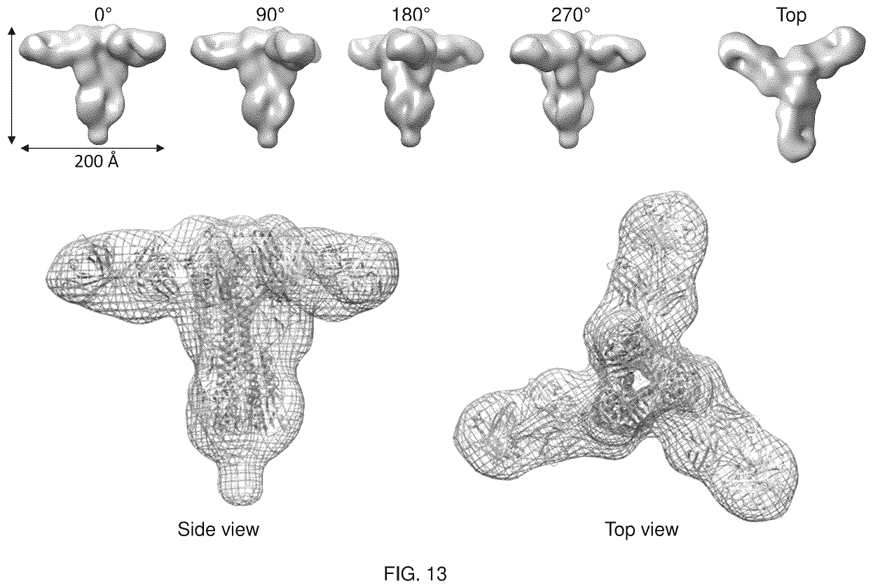

Serum samples were collected prior to the first immunization and at two and three weeks after the second immunization for serological analyses. More specifically the immune sera were tested for their ability to inhibit hemagglutination mediated by NC99 virus and neutralize NC99 pseudotyped lentivirus The results of these analyses are shown in FIGS. 7 and 8.

### Example 4. Analysis of Breadth of Immune Response Using Monovalent Nanoparticles, Admixed Monovalent Nanoparticles or Multivalent, Co-Assembled Nanoparticles

Mice (N=5) were immunized with either monovalent nanoparticles against NC99 or CA09, admixed nanoparticles (Admix 4), or multivalent nanoparticles (CoAsmbl 4 or CoAsmbl 8) (N=5) at week 0 and again at week 3. At 2-3 weeks following the second immunization, sera was collected from each mouse and the sera analyzed by HAI assays using a panel of 18 H1N1 viruses. The resulting titers are shown as a heatmap in FIG. 9.

The results of this analysis demonstrate that immunization with multivalent co-assembled particles produces a broader immune response (i.e., an immune response against a wider range of influenza viruses) than does immunization with either monovalent nanoparticles or admixed monovalent nanoparticles.

### Example 5. Detection of HA-Specific, Cross-Reactive B-Cells in Peripheral Cells in HA RBD-Nanoparticle Immunized Mice

Mice (N=5) were immunized with either monovalent nanoparticles against NC99 or CA09, admixed nanoparticles (Admix 2, Admix 4, or Admix 6), or multivalent nanoparticles (CoAsmbl 2, CoAsmbl 4, CoAsmbl 6 or CoAsmbl 8) at week 0, 3 and 20. At 10 days following the third immunization, peripheral blood was collected from each mouse and the white blood cells were isolated and analyzed by flow cytometer using NC99 and CA09 HA probes. Live, non-T, non-mactophage, IgD negative, singlet memory B cells were gated and the cell population (percentage of memory B cells) positive to both NC99 and CA09 HA was quantitated. The gating strategy and the resulting frequency of HA double positive cells across different immunization groups are shown in FIGS. 10 and 11.

The results of this analysis demonstrate that immunization with multivalent co-assembled particles induces an increased frequency of cross-reactive HA-specific memory B cells in immunized animals (i.e., B cells specific for both NC99 and CA09 HA) than does immunization with either monovalent nanoparticles or admixed monovalent nanoparticles.

### Example 6. Correlation of NC99/CA09 Cross-Reactive B-Cell Frequency and Antigenic Heterogeneity of Co-Assembled RDP-Nanoparticle

Relationship between frequency of HA double positive cells in immunized animals and antigen valence of co-assembled immunogens was examined by Pearson product moment correlation analysis. This relationship is illustrated by the graph in FIG. 12.

The result of this analysis show that the degree of heterogeneity on the co-assembled immunogens positively correlates with frequency of cross-reactive HA-specific memory B cells in immunized animal.

### Example 7. Neutralization Breadth Elicited by Vaccination with Co-Assembled HA RBD-Nanoparticles

Mice were vaccinated with co-assembled nanoparticles, according to the schedule shown above in Table 3. Ten days after the final immunization, sera were collected and tested for its ability to neutralize pseudotyped lentiviruses expressing HA and NA from various H1N1 virus strains. The serum neutralization titers obtained from these assays are shown below in Table 4.

TABLE 4
Serum neutralization titers from mice
immunized with co-assembled nanoparticles

| | IC₅₀ (serum dilution) | | | | |
|---|---|---|---|---|---|
| H1N1 pseudovirus | #8441 | #8442 | #8443 | #8444 | #8445 |
| A/California/4/09 | NT | NT | NT | NT | NT |
| A/New Jersey/76 | 2528 | 947 | 27580 | 11222 | 7463 |
| A/South Carolina/1/18 | 1146 | 242 | 1354 | 11129 | 217 |
| A/Wilson-Smith/33 | NT | NT | NT | NT | NT |
| A/Puerto Rico/8/34 | 5439 | <40 | 3440 | 9822 | 2817 |
| A/Iowa/43 | 3062 | 1709 | 12821 | 12095 | 2466 |
| A/Fort Monmouth/1/47 | 31302 | 85350 | 77846 | 126781 | 719 |
| A/Malaysia/54 | NT | NT | NT | NT | NT |
| A/Albany/4835/48 | NT | NT | NT | NT | NT |
| A/Hong Kong/117/77 | 2636 | 7525 | 16876 | 37845 | 1724 |
| A/Singapore/6/86 | 8888 | 4064 | 9822 | 3469 | 24751 |
| A/New York/146/00 | 13366 | 7780 | 6866 | 3113 | 15399 |
| A/New York/653/96 | 17302 | 158 | 9190 | 5349 | 22212 |
| A/Beijing/262/95 | 5960 | <40 | 1905 | 9422 | 96 |
| A/New | 5217 | 15788 | 18649 | 42171 | 46992 |
| Caledonia/20/99 | | | | | |
| A/New York/8/06 | 1695 | 423 | 947 | 620 | 2317 |
| A/Solomon | 1072 | 604 | 226 | 1072 | 1858 |
| Islands/3/06 | | | | | |
| A/Brisbane/59/07 | NT | NT | NT | NT | NT |

NT, not tested.

### Example 8. Neutralization Breadth of an Isolated, Anti-HA Monoclonal Antibody

B cells obtained from mouse #8441 in Example 7 were sorted using fluorescently labeled HA probe as bait. Genes encoding variable regions of the antibody heavy and light chains were then amplified from single B cells, sequenced, and cloned into appropriate backbone vectors (mouse IgG2a heavy and kappa light chain backbone) to express the encoded proteins as an antibody. Reconstructed antibody vectors were used for transient transfection in 293-Freestyle expression system (Life technologies) and the IgG was purified by affinity column purification using protein A resin. The resulting antibody was referred to as 441D6. Neutralization IC₅₀ titers of 441D6 were determined by lentivirus pseudotype neutralization assays in which pseudoviruses express HA and NA from various H1N1 viral strains. Monoclonal antibodies CH65 (anti-receptor binding site of HA) and FI6v3 (anti-HA stem region) were used as controls. NT=not tested. The neutralization titers obtained from these assays are shown below in Table 5.

TABLE 5
Neutralization titers of monoclonal antibody 441D6

| | IC₅₀ (μg/ml) | | |
|---|---|---|---|
| H1N1 pseudovirus | CH65 | 441D6 | FI6v3 |
| A/California/4/09 | 6.25 | 0.16 | 0.13 |
| A/New Jersey/76 | >25 | 0.03 | 0.36 |
| A/South Carolina/1/18 | 0.57 | 0.04 | 4.40 |
| A/Wilson-Smith/33 | NT | NT | NT |
| A/Puerto Rico/8/34 | 0.44 | 0.08 | 0.97 |
| A/Iowa/43 | >50 | 11.08 | 3.40 |
| A/Fort Monmouth/1/47 | 5.86 | 0.02 | 22.17 |
| A/Malaysia/54 | NT | NT | NT |
| A/Albany/4835/48 | NT | NT | NT |
| A/Hong Kong/117/77 | 0.97 | 0.04 | 0.09 |
| A/Singapore/6/86 | <0.005 | 0.01 | 0.01 |
| A/New York/146/00 | 0.03 | 0.01 | 0.08 |
| A/New York/653/96 | <0.005 | 0.02 | 0.15 |
| A/Beijing/262/95 | 0.01 | 0.07 | 1.01 |
| A/New | 0.01 | 0.04 | 0.09 |
| Caledonia/20/99 | | | |
| A/New York/8/06 | 0.02 | 0.14 | >25 |
| A/Solomon | 1.30 | 0.29 | 0.94 |
| Islands/3/06 | | | |
| A/Brisbane/59/07 | NT | NT | NT |

NT, not tested.

The results demonstrate the ability of the monoclonal antibody 441D6 to neutralize broader range of H1N1 viruses than CH65 and more potently neutralize viruses than FI6v3, documenting a novel broad and potent neutralizing monoclonal antibody 441D6 against H1N1 viruses.

To better understand the interaction of monoclonal antibody 441D6 with influenza HA protein, a three-dimensional reconstruction model of an HA trimer complexed with Fab 441D6 was produced. Briefly, HA trimer (A/New York/653/1996 (H1N1)) was incubated with 1.5 times excess amount of Fab 441D6 and the complex purified by size exclusion column chromatography. The purified HA-Fab complex was then used in negative stain electron microscopy experiments. Approximately 9,000 particles were used for three dimensional reconstruction and the calculated resolution of the final model was ˜18.5 Å. HA and Fab models were docked in the EM density (bottom). The resulting three-dimensional model is shown in FIG. 13.

What is claimed:

1. A method of producing a nanoparticle, comprising:
culturing a cell comprising at least two nucleic acid molecules, wherein:
each nucleic acid molecule encodes a unique species of self-assembling fusion protein, each fusion protein comprises a self-assembling, monomeric subunit protein joined to an immunogenic portion of an influenza virus hemagglutinin (HA) protein, wherein the immunogenic portion of the influenza virus HA protein is a receptor binding domain (RBD);
the immunogenic portion in each species of self-assembling fusion protein differs from the immunogenic portion in the other species of self-assembling fusion proteins by at least one amino acid;
the fusion proteins self-assemble to form a nanoparticle that displays on its surface, the immunogenic portions of the at least two species of self-assembling fusion proteins;
at least one of the species of self-assembling fusion proteins comprises an amino acid sequence at least 80% identical to SEQ ID NO: 97; and
the cell is cultured under conditions suitable for expressing the encoded self-assembling fusion proteins to form the nanoparticle.

2. The method of claim 1, wherein at least one of the species of self-assembling fusion proteins comprises an amino acid sequence at least 90% identical to SEQ ID NO: 97.

3. The method of claim 1, wherein at least one of the species of self-assembling fusion proteins comprises an amino acid sequence at least 95% identical to SEQ ID NO: 97.

4. The method of claim 1, wherein at least one of the species of self-assembling fusion proteins comprises an amino acid sequence at least 99% identical to SEQ ID NO: 97.

5. The method of claim 1, wherein the at least one species of self-assembling fusion protein comprises an amino acid sequence set forth as SEQ ID NO:97, SEQ ID NO:100, SEQ ID NO:103, SEQ ID NO: 106, SEQ ID NO:109, SEQ ID NO:112, SEQ ID NO:115, SEQ ID NO:118, SEQ ID NO:121, SEQ ID NO:124, SEQ ID NO:127, SEQ ID NO:130, SEQ ID NO:133, SEQ ID NO:136, SEQ ID NO:139, or SEQ ID NO:142.

6. The method of claim 1, wherein the at least one species of self-assembling fusion protein comprises an amino acid sequence set forth as SEQ ID NO: 97.

7. A method of producing a nanoparticle, comprising:
culturing a cell comprising at least four nucleic acid molecules, wherein:
each nucleic acid molecule encodes a unique species of self-assembling fusion protein, each fusion protein comprises a self-assembling, monomeric subunit protein joined to an immunogenic portion of an influenza virus hemagglutinin (HA) protein, wherein the immunogenic portion of the influenza virus HA protein is a receptor binding domain (RBD);
the immunogenic portion in each species of self-assembling fusion protein differs from the immunogenic portion in the other species of self-assembling fusion proteins by at least one amino acid;
the fusion proteins self-assemble to form a nanoparticle that displays on its surface, the immunogenic portions of the at least four species of self-assembling fusion proteins;
at least one of the species of self-assembling fusion proteins comprises an amino acid sequence at least 80% identical to SEQ ID NO: 97; and
the cell is cultured under conditions suitable for expressing the encoded self-assembling fusion proteins to form the nanoparticle.

8. The method of claim 7, wherein at least one of the species of self-assembling fusion proteins comprises an amino acid sequence at least 90% identical to SEQ ID NO: 97.

9. The method of claim 7, wherein at least one of the species of self-assembling fusion proteins comprises an amino acid sequence at least 95% identical to SEQ ID NO: 97.

10. The method of claim 7, wherein at least one of the species of self-assembling fusion proteins comprises an amino acid sequence at least 99% identical to SEQ ID NO: 97.

11. The method of claim 7, wherein the at least one species of self-assembling fusion protein comprises an amino acid sequence set forth as SEQ ID NO:97, SEQ ID NO:100, SEQ ID NO:103, SEQ ID NO: 106, SEQ ID NO: 109, SEQ ID NO:112, SEQ ID NO:115, SEQ ID NO:118, SEQ ID NO:121, SEQ ID NO:124, SEQ ID NO:127, SEQ ID NO:130, SEQ ID NO:133, SEQ ID NO:136, SEQ ID NO:139, or SEQ ID NO:142.

12. The method of claim 7, wherein the at least one species of self-assembling fusion protein comprises an amino acid sequence set forth as SEQ ID NO: 97.
